(19) United States
(12) Patent Application Publication
(10) Pub. No.: US 2007/0179155 A1
(43) Pub. Date: Aug. 2, 2007

(54) N-phenyl-piperazine derivatives and methods of prophylaxis or treatment of 5ht2c receptor associated diseases

(76) Inventors: Brian Smith, San Diego, CA (US); Tsai James, San Diego, CA (US); Chen Rita, Los Angeles, CA (US)

Correspondence Address: ARENA PHARMACEUTICALS, INC., 6166 NANCY RIDGE DRIVE, SAN DIEGO, CA, 92121, US

(21) Appl. No.: 10/561,101

(22) PCT Filed: Jun. 17, 2004
(86) PCT No.: PCT/US04/19540

§ 371 Date: May 26, 2006

Related U.S. Application Data
(60) Provisional application No. 60/480,045, filed on Jun. 20, 2003.

(51) Int. Cl. A61K 31/495; C07D 241/04

(52) U.S. Cl. 514/255.03; 544/392

(57) ABSTRACT

The present invention relates to certain substituted N-phenyl-piperazine derivatives of Formula: (I) that are modulators of the 5HT_{2c} receptor. Accordingly, compounds of the present invention are useful for the prophylaxis or treatment of 5HT_{2c} receptor associated diseases or disorders, such as, obesity, Alzheimer Disease, erectile dysfunction and related disorders.

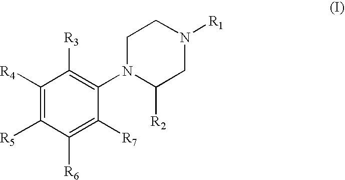

### FIELD OF THE INVENTION

The present invention relates to certain substituted N-phenyl-piperazine derivatives that are modulators of the 5HT_{2C} receptor. Accordingly, compounds of the present invention are useful for the prophylaxis or treatment of 5HT_{2C} receptor associated diseases or disorders, such as, obesity, Alzheimer Disease, erectile dysfunction and related disorders.

### BACKGROUND OF THE INVENTION

Obesity is a life-threatening disorder in which there is an increased risk of morbidity and mortality arising from concomitant diseases such as, but not limited to, type II diabetes, hypertension, stroke, certain forms of cancers and gallbladder disease.

Obesity has become a major healthcare issue in the Western World and increasingly in some third world countries. The increase in the number of obese people is due largely to the increasing preference for high fat content foods but also, and this can be a more important factor, the decrease in activity in most people's lives. In the last 10 years there has been a 30% increase in the incidence of obesity in the USA and that about 30% of the population of the USA is now considered obese. In spite of the growing awareness of the health concerns linked to obesity the percentage of individuals that are overweight or obese continue to increase. In fact, the percentage of children and adolescents who are defined as overweight has more than doubled since the early 1970s and about 13 percent of children and adolescents are now seriously overweight. The most significant concern, from a public health perspective, is that children who are overweight grow up to be overweight or obese adults, and accordingly are at greater risk for major health problems. Therefore, it appears that the number of individuals that are overweight or obese will continue to increase.

Whether someone is classified as overweight or obese is generally determined on the basis of his or her body mass index (13MI) which is calculated by dividing their body weight (kilograms—Kg) by their height squared (meters squared—m²). Thus, the units for BMI are Kg/m². The BMI is more highly correlated with body fat than any other indicator of height and weight. A person is considered overweight when they have a BMI in the range of 25-30 kg/n². Whereas a person with a BMI over 30 kg/m² is classified as obese and obesity is further divided into three classes, Class I (BMI of about 30 to about 34.9 kg/m²), Class II (BMI of about 35 to 39.9 kg/m²) and Class III (about 40 kg/m² or greater); see TABLE 1 below for complete classifications.

TABLE 1
CLASSIFICATION OF WEIGHT
BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases for an individual there is an increased risk of morbidity and mortality relative to an individual with normal BMI. Accordingly, overweight and obese individuals (BMI of about 25 kg/m² and above) are at increased risk for physical ailments such as, but not limited to, high blood pressure, cardiovascular disease (particularly hypertension), high blood cholesterol, dyslipidemia, type II (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), diseases of reproduction (such as sexual dysfunction, both male and female, including male erectile dysfunction), bladder control problems (such as stress incontinence), uric acid nephrolithiasis, psychological disorders (such as depression, eating disorders, distorted body image, and low self esteem). Research has shown that even a modest reduction in body weight can correspond to a significant reduction in the risk of developing other ailments, such as, but not limited to, coronary heart disease.

As mentioned above, obesity increases the risk of developing cardiovascular diseases. Coronary insufficiency, atheromatous disease, and cardiac insufficiency are at the forefront of the cardiovascular complications induced by obesity. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of obesity [Perry, I. J., et al. BMJ 310, 560-564 (1995)]. It is estimated that if the entire population had an ideal weight, the risk of coronary insufficiency would decrease by 25% and the risk of cardiac insufficiency and of cerebral vascular accidents by 35%.

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina and increases the risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

The first line of treatment for individuals that are overweight or obese is to offer diet and life style advice, such as, reducing the fat content of their diet and increasing their physical activity. However many patients find these difficult to maintain and need additional help from drug therapy to sustain results from these efforts.

Most currently marketed products have been unsuccessful as treatments for obesity owing to a lack of efficacy or unacceptable side-effect profiles. The most successful drug so far was the indirectly acting 5-hydroxytryptarnine (5-HT) agonist d-fenfluramine (Redux™) but reports of cardiac valve defects in up to one third of the patient population led to its withdrawal by the FDA in 1998.

In addition, two drugs have recently been launched in the USA and Europe: Orlistat (Xenical™), a drug that prevents absorption of fat by the inhibition of pancreatic lipase, and Sibutramine (Reductil™), a 5-HT/noradrenaline re-uptake inhibitor. However, side effects associated with these products may limit their long-term utility. Treatment with Xenical™ is reported to induce gastrointestinal distress in some patients, while Sibutramine has been associated with raised blood pressure in some patients.

Serotonin (5-HT) neurotransmission plays an important role in numerous physiological processes both in health and in psychiatric disorders. 5-HT has been implicated in the regulation of feeding behavior for some time. 5-HT works by inducing a feeling of fullness or satiety so eating stops earlier and fewer calories are consumed. It has been shown that a stimulatory action of 5-HT on the 5HT_{2C} receptor plays an important role in the control of eating and in the anti-obesity effect of d-fenfluramine. As the 5HT_{2C} receptor is expressed in high density in the brain (notably in the limbic structures, extrapyramidal pathways, thalamus and hypothalamus i.e. PVN and DMH, and predominantly in the choroid plexus) and is expressed in low density or is absent in peripheral tissues, a selective 5HT_{2C} receptor agonist can be an effective and safe anti-obesity agent. Also, 5HT_{2C} knockout mice are overweight with cognitive impairment and susceptibility to seizure thus establishing the clear use for a 5HT_{2C} receptor agonist in 5HT_{2C} receptor associated diseases or disorders.

The 5HT_{2C} receptor plays a role in obsessive compulsive disorder, some forms of depression, and epilepsy. Accordingly, 5HT_{2C} receptor agonists can have anti-panic properties, and properties useful for the treatment of sexual dysfunction. In addition, 5HT_{2C} receptor agonists are useful for the treatment of psychiatric symptoms and behaviors in individuals with eating disorders such as, but not limited to, anorexia nervosa and bulimia nervosa. Individuals with anorexia nervosa often demonstrate social isolation. Anorexic individuals often present symptoms of being depressed, anxious, obsession, perfectionistic traits, and rigid cognitive styles as well as sexual disinterest. Other eating disorders include, anorexia nervosa, bulimia nervosa, binge eating disorder (compulsive eating) and ED-NOS (i.e., eating disorders not otherwise specified—an official diagnosis). An individual diagnosed with ED-NOS possess atypical eating disorders including situations in which the individual meets all but a few of the criteria for a particular diagnosis. What the individual is doing with regard to food and weight is neither normal nor healthy.

In addition, the 5HT_{2C} receptor is also involved in other diseases, conditions and disorders; such as Alzheimer Disease (AD). Therapeutic agents currently prescribed for Alzheimer's disease (AD) are cholinomimetic agents that act by inhibiting the enzyme acetylcholinesterase. The resulting effect is increased levels of acetylcholine, which modestly improves neuronal function and cognition in patients with AD. Although, dysfunction of cholinergic brain neurons is an early manifestation of AD, attempts to slow the progression of the disease with these agents have had only modest success, perhaps because the doses that can be administered are limited by peripheral cholinergic side effects, such as tremors, nausea, vomiting, and dry mouth. In addition, as AD progresses, these agents tend to lose their effectiveness due to continued cholinergic neuronal loss.

Therefore, there is a need for agents that have beneficial effects in AD, particularly in alleviating symptoms by improving cognition and slowing or inhibiting disease progression, without the side effects observed with current therapies. Therefore, serotonin 5HT_{2C} receptors, which are exclusively expressed in brain, are attractive targets.

A major feature of AD is the formation of senile plaques made of amyloid deposits in a selected area of the brain. New therapies should focus on prevention of the production of these senile plaques. An amyloid deposit composed mainly of beta-amyloid peptide (Aβ) occupies the plaque center. Aβ is a peptide of 40 to 43 residues derived from a larger amyloid precursor protein, APP [Selkoe D J, et al. Ann Rev Neurosci, 1994, 17:489-517]. APP is a ubiquitous transmembrane glycoprotein that is present at high levels in brain cells. APP also exists as secreted forms. By cleavage in the Aβ region of APP, the long N-terminal fragment (secreted APP, APPs) is secreted into the extracellular space. The rate of Aβ production appears to be inversely coupled to rate APPs secretion. In several cell cultures, APPs secretion was accompanied by reductions in secreted Aβ [Buxbaum J D, et al. Proc Nat Acad Sci, 1993, 90:9195-9198; Gabuzda D, et al. J Neurochem, 1993, 61:2326-2329; Hung A Y, et al. J Biol Chem, 1993, 268:22959-22962; and Wolf B A, et al. J Biol Chem, 1995, 270:4916-4922], suggesting that stimulated secretory processing of APP into secreted APPs is associated with reduced formation of potentially amyloidogenic derivatives, or plaques.

APPs is found in plasma and cerebrospinal fluid [Ghiso J, et al. Biochem Biophys Res Comm, 1989, 163:430-437; and Podlisny M B, et al. Biochem Biophys Res Commun, 1990, 167:1094-1101]. Considering the abundance of both membrane-bound APP and APPs, they are likely to have significant biological functions. Current knowledge about APP functions indicates APP is critically required for the maintenance of neuronal and synaptic structure and function. Membrane-bound APP has been suggested to have a receptor-like structure [Kang J, et al. Nature, 1987, 325:733-736], with the cytoplasmic domain capable of complexing with a GTP-binding protein [Nishimoto I., et al. Nature, 1993, 362:75-79]. Membrane-embedded full-length APP might also have a cell adhesion function [Qiu W., et al. J Neurosci, 1995, 15:2157-2167].

APPs has been shown to be neurotrophic and neuroprotective in vitro [Mattson M P, et al. Neuron, 1993, 10:243-254; and Qiu W., et al. J Neurosci, 1995, 15:2157-2167]. Other proposed functions for APPs include the regulation of blood coagulation [Cole G M, et al. Biochem Biophys Res Commun, 1990, 170:288-295; Smith R P, et al. Science, 1990, 248:1126-1128; and Van Nostrand et al. Science, 1990, 248:745-748], wound-healing [Cunningham J M, et al. Histochemistry, 1991, 95:513-517], extracellular protease activity [Oltersdorf T, et al. Nature (London), 1989, 341:144-147; and Van Nostrand W E, et al. Nature, 1989, 341:546-548], neurite extension [Jin L., et al. J Neurosci, 1994, 14:5461-5470; and Robakis N K, et al. in Molecular Biology of Alzheimer's Disease. (T. Miyatake, D. J. Selkoe and Y. Ihara, ed.), 1990, pp. 179-188, Elsevier Science Publishers B.V., Amsterdam], cell adhesiveness [Schubert D, et al. Neuron, 1989, 3:689-694], cell growth, [Bhasin R., et al. Proc Natl Acad Sci USA, 1991, 88:10307-10311; and Saitoh T., Cell, 1989, 58:615-622], and differentiation [Araki W., et al. Biochem Biophys Res Commun, 1991, 181:265-271; Milward E A, et al. Neuron, 1991, 9:129-137; and Yamamoto K, et al. J Neurobiol, 1994, 25:585-594].

The non-selective serotonin 5HT_{2C} agonist dexnorfenfluramine (DEXNOR) stimulated amyloid precursor protein (APPs) secretion in guinea pigs while reducing levels of Aβ production in vivo following repeat administration [Arjona A, et al. “Effect of a 5HT_{2C} serotonin agonist, dexnorfenfluramine, on amyloid precursor protein metabolism in guinea pigs,” Brain Res, 2002, 951:135-140]. Guinea pigs were chosen because guinea pig and human APP exhibit 98% sequence homology [Beck M, et al. Biochem Biophys Acta, 1997, 1351:17-21], the proteins are processed similarly [Beck M., et al. Neuroscience, 1999, 95:243-254], and the Aβ peptide sequences are identical [Johnstone E M, et al. Brain Res Mol Brain Res, 1991, 10:299-305]. Although DEXNOR is non-selective, the observed effects were attenuated by a selective serotonin 5HT_{2C} antagonist, while a selective serotonin HT_{2A} antagonist did not reverse the DEXNOR effects, indicating the serotonin 5HT_{2C} receptors are the most relevant target for this effect.

In addition, 5-HT stimulates APPs ectodornain secretion via the serotonin 5HT_{2A} and 5HT_{2C} receptors [Nitsch R M, et al. J Biol Chem, 1996, 271(8):4188-4194]. In this study, researchers stimulated 3T3 fibroblasts with serotonin (5-HT), which were stably expressing serotonin 5HT_{2A} or 5HT_{2C} receptors. 5-HT increased APPs secretion in a dose-dependent manner in both cell lines. Maximal stimulation of APPs secretion peaked at about 4-fold. Selective serotonin 5HT_{2A} and 5HT_{2C} antagonists blocked the effects in each cell line.

A serotonin 5HT_{2C} receptor agonist can be effective for treating AD and preventing senile plaques. Support for this claim comes from the fact that Aβ is known to be neurotoxic and a key component in senile plaques involved in AD, APPs secretion and Aβ levels seem to be inversely related, and serotonin 5HT_{2C} agonists increase levels of APPs in vitro in cell lines stably expressing serotonin 5HT_{2C} receptors while in vivo serotonin 5HT_{2C} agonists increase levels of APPs and decrease levels of Aβ as measured in cerebral spinal fluid of guinea pigs.

Evidence exists supporting the use of a compound of the present invention with agonist activity at the serotonin 5HT_{2C} receptor for the treatment of AD. The compound of the invention can be used alone or in combination with another agent or agents (such as but not limited to AChE inhibitors) that are typically prescribed for AD.

Another disease, disorder or condition that can is associated with the function of the 5HT_{2C} receptor is erectile dysfunction (ED). Erectile dysfunction is the inability to achieve or maintain an erection sufficiently rigid for intercourse, ejaculation, or both. An estimated 20-30 million men in the United States have this condition at some time in their lives. The prevalence of the condition increases with age. Five percent of men 40 years of age report ED. This rate increases to between 15% and 25% by the age of 65, and to 55% in men over the age of 75 years.

Erectile dysfunction can result from a number of distinct problems. These include loss of desire or libido, the inability to maintain an erection, premature ejaculation, lack of emission, and inability to achieve an orgasm. Frequently, more than one of these problems presents themselves simultaneously. The conditions may be secondary to other disease states (typically chronic conditions), the result of specific disorders of the urogenital system or endocrine system, secondary to treatment with pharmacological agents (e.g. antihypertensive drugs, antidepressant drugs, antipsychotic drugs, etc.) or the result of psychiatric problems. Erectile dysfunction, when organic, is primarily due to vascular irregularities associated with atherosclerosis, diabetes, and hypertension.

There is evidence for use of a serotonin 5HT_{2C} agonist for the treatment of sexual dysfunction in males and females. The serotonin 5HT_{2C} receptor is involved with the processing and integration of sensory information, regulation of central monoamninergic systems, and modulation of neuroendocrine responses, anxiety, feeding behavior, and cerebrospinal fluid production [Tecott, L. H., et al. Nature 374: 542-546 (1995)]. In addition, the serotonin 5HT_{2C} receptor has been implicated in the mediation of penile erections in rats, monkeys, and humans.

The exact mechanism by which 5HT_{2C} receptors mediate penile erections remains unknown. However, there is good evidence, indirect and direct, supporting the role of serotonin 5HT_{2C} receptors in the mediation of penile erections. Anatomical studies have shown that the penis receives autonomic innervation from sympathetic and parasympathetic nuclei located in the spinal cord [Pescatori E S, et al. J Urol 1993; 149: 627-32]. In agreement, experimental and clinical data support that penile erections are controlled by a spinal reflex. A closer analysis showed that activation of 5HT₂ spinal receptors facilitated pudendal reflex in anesthetized cats [Danuser H and Thor K B, Br J Pharmacol 1996; 118: 150-4]. Accordingly, stimulation of 5HT_{2C} receptors has been shown to be proerectile [Millan E J, et al. European Journal of Pharmacology 1997; 325], and 5HT_{2C} receptors have been described on proerectile spinal parasympathetic neurons [Bancila M et al. Neuroscience 1999; 92: 1523-37].

Indirect evidence comes from the research and reports of the side effects induced by the use of selective serotonin reuptake inhibitors (SSRIs). SSRIs have demonstrated antagonist action at the serotonin 5HT_{2C} receptors [Jenck et al. European Journal of Pharmacology 231: 223-229 (1993); Lightlowler et al. European Journal of Pharmacology 296: 137-43 (1996); and Palvimaki, E., et al. Psychopharmacology 126: 234-240 (1996)]. Among the most derogatory side effects of SSRIs noted in humans is increased difficulty in attaining penile erection. Although SSRIs have a rich pharmacological profile, it is believed that the antagonist effects of SSRIs at the 5HT_{2C} receptors could be implicated in the inhibition of penile erections [Palvimaki, E., et al. Psychopharmacology 126: 234-240 (1996)].

Further evidence comes from studies with a variety compounds with known agonist activity for the serotonin 5HT_{2C} receptor. Pharmacologic studies with rats and rhesus monkeys provide direct evidence of the proerectile properties of agonist of the serotonin 5-HT_{2C} receptor [Millan M J, et al. European Journal of Pharmacology 1997; 325; and Pomerantz, et al. European Journal of Pharmacology 243:227-34 (1993)]. These pro-erectile effects were unaffected by antagonists for the serotonin 5HT_{2A} and 5HT_{2B} receptors, respectively. Antagonists of the serotonin 5HT_{2C} receptors attenuated the proerectile effects of the 5-HT_{2C} agonists. The inhibition action corresponded to each antagonist's affinity for the 5-HT_{2C} receptors. In addition, agonists of the serotonin 5HT_{2A} and 5HT_{2B} receptors did not elicit penile erections.

In summary, the 5HT_{2C} receptor is a validated and well-accepted receptor target for the prophylaxis and/or treatment of 5HT_{2C} mediated receptor diseases and disorders, such as, obesity, eating disorders, psychiatric disorders, Alzheimer Disease, sexual dysfunction and disorders related thereto. It can be seen that there exists a need for selective 5HT_{2C} receptor agonists that can safely address these needs. The present invention is directed to these, as well as other, important ends.

### SUMMARY OF THE INVENTION

The present invention is drawn to compounds which bind to and modulate the activity of the 5HT_{2C} receptor, and uses thereof. The term 5HT_{2C} receptor as used herein includes the human sequences found in GeneBank accession number AF498983, naturally-occurring allelic variants, mammalian orthologs, and recombinant mutants thereof.

One aspect of the present invention pertains to certain substituted N-phenyl-piperazine derivatives as represented by Formula (I):

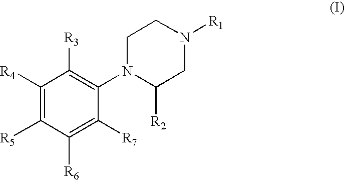

wherein:

R₁ is H or C₁₋₈ alkyl;

R₂ is C₂₋₄ alkenyl, C₁₋₄ alkyl or C₁₋₄ haloalkyl; and R₃, R₄, R₅, R₆ and R₇ are each independently H, C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl, phenyl, and thiol; or

a pharmaceutically acceptable salt, hydrate and solvate thereof.

In some embodiments, the compound is not 1-(4-Chloro-phenyl)-2-methyl-piperazine; 1-(3,5-Difluoro-phenyl)-2-methyl-piperazine; 2-Methyl-1-(2-methylsulfanyl-phenyl)-piperazine; 4-Amino-3-fluoro-2-(2-methyl-piperazin-1-yl)-5-nitro-benzonitrile; 2-Methyl-1-phenyl-piperazine; 4-(2-Isopropyl-piperazin-1-yl)-2-trifluoromethyl-benzonitrile; 4-(2-Ethyl-piperazin-1-yl)-2-trifluoromethyl-benzonitrile; 4-(2-Methyl-piperazin-1-yl)-2-trifluoromethyl-benzonitrile; 1-(3-Chloro-phenyl)-2-methyl-piperazine; 2-Methyl-1-m-tolyl-piperazine; 4-(2-Methyl-piperazin-1-yl)-benzamide; 1-(2-Fluoro-phenyl)-2-methyl-piperazine; 4-(2-Methyl-piperazin-1-yl)-phenol; 1-(3-Methoxy-phenyl)-2-methyl-piperazine; 2-Methyl-1-(3-trifluoromethyl-phenyl)-piperazine; 1-(4-Methoxy-phenyl)-2-methyl-piperazine; 2-Methyl-1-p-tolyl-piperazine; 2,4-Dimethyl-1-phenyl-piperazine; 4-Chloro-5-(4-ethyl-2-methyl-piperazin-1-yl)-benzene-1,2-diamine; 4-Chloro-5-(4-ethyl-2-methyl-piperazin-1-yl)-2-nitro-phenylamine; 5-(4-Ethyl-2-methyl-piperazin-1-yl)-2-nitro-4-trifluoromethyl-phenylamine; and 5-(4-Ethyl-2-methyl-piperazin-1-yl)-4-methyl-2-nitro-phenylamine.

Some embodiments of the present invention are compounds of Formula (I) wherein the compounds are the R enantiomers.

Some embodiments of the present invention are compounds of Formula (I) wherein the compounds are the S enantiomers.

Another aspect of the present invention pertains to pharmaceutical compositions comprising a pharmaceutical acceptable carrier in combination with at least one compound according to Formula (I).

Another aspect of the present invention pertains to methods of modulating a 5HT_{2C} receptor comprising contacting said receptor with a therapeutically effective amount or dose of a compound as described herein. Preferably, compounds of the present invention are agonists of the 5HT_{2C} receptor.

Another aspect of the present invention pertains to methods of prophylaxis or treatment of disorders of the central nervous system; damage to the central nervous system; cardiovascular disorders; gastrointestinal disorders; diabetes insipidus or sleep apnea comprising administering to an individual in need of such prophylaxis or treatment a therapeutically effective amount or dose of a compound of the present invention or a pharmaceutical composition thereof. In some embodiments, the individual is a mammal. Preferably, the mammal is a human.

Another aspect of the present invention pertains to methods of decreasing food intake of an individual comprising administering to said individual a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of inducing satiety in an individual comprising administering to said individual a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of controlling weight gain of an individual comprising administering to said individual suffering from weight control a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of producing a pharmaceutical composition comprising admixing at least one compound of the present invention and at least one pharmaceutically acceptable carrier.

Another aspect of the present invention pertains to compounds, as described herein, for use in a method of treatment of the human or animal body by therapy.

Another aspect of the present invention pertains to compounds, as described herein, for use in a method of prophylaxis or treatment of disorders of the central nervous system; damage to the central nervous system; cardiovascular disorders; gastrointestinal disorders; diabetes insipidus or sleep apnea of the human or animal body by therapy.

Another aspect of the present invention pertains to use of compounds, as described herein, for the manufacture of a medicament for use in the treatment or prophylaxis of disorders of the central nervous system; damage to the central nervous system; cardiovascular disorders; gastrointestinal disorders; diabetes insipidus or sleep apnea.

In some embodiments, the disorders of the central nervous system are selected the group consisting of depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension. In further embodiments, the disorder of the central nervous system is obesity. En further embodiments, the disorder of the central nervous system is Alzheimer disease. In further embodiments, the sexual dysfunction is Male erectile dysfunction.

In some embodiments, the damage to the central nervous system is by trauma, stroke, neurodegenerative diseases, toxic CNS diseases or infective CNS diseases. In further embodiments, the damage to the central nervous system is by encephalitis or meningitis.

In some embodiments, the cardiovascular disorder is thrombosis. In some embodiments, the gastrointestinal disorder is dysfunction of gastrointestinal motility.

In some embodiments, the invention pertains to methods for alleviation of a symptom of any of the diseases, conditions or disorders mentioned herein.

This application is related to U.S. Provisional Patent Application, Ser. No. 60/480,045, which is incorporated by reference in its entirety.

Applicant reserves the right to exclude any one or more of the compounds from any of the embodiments of the invention. Applicant additionally reserves the right to exclude any disease, condition or disorder from any of the embodiments of the invention.

### BRIEF DESCRIPTION OF THE FIGURES

FIG. 1 shows the effects of Compound 44 of the present invention on basal food intake in rats. The ED₅₀s (μmol/kg, p.o.) for Compound 44 were determined at 2, 4, 6, and 22 hours after food presentation to be 33, 58, 97, and 441, respectively.

### DETAILED DESCRIPTION OF THE INVENTION

Definitions

For clarity and consistency, the following definitions will be used throughout this patent document.

AGONISTS shall mean moieties that interact and activate the receptor, such as the 5HT_{2c} receptor and initiates a physiological or pharmacological response characteristic of that receptor. For example, when moieties activate the intracellular response upon binding to the receptor, or enhance GTP binding to membranes.

The term ANTAGONISTS is intended to mean moieties that competitively bind to the receptor at the same site as agonists (for example, the endogenous ligand), but which do not activate the intracellular response initiated by the active form of the receptor, and can thereby inhibit the intracellular responses by agonists or partial agonists. Antagonists do not diminish the baseline intracellular response in the absence of an agonist or partial agonist.

Chemical Group, Moiety or Radical:

The term “C₁₋₄ acyl” denotes an alkyl radical attached to a carbonyl wherein the definition of alkyl has the same definition as described herein; some examples include formyl, acetyl, propionyl, butanoyl, iso-butanoyl, and the like.

The term “C₁₋₄ acyloxy” denotes an acyl radical attached to an oxygen atom wherein acyl has the same definition has described herein; some examples include acetyloxy, propionyloxy, butanoyloxy, iso-butanoyloxy and the like.

The term “C₁₋₄ acylthioxy” denotes a thioacyl [i.e., alkyl-C(═S)—] radical attached to an oxygen atom; some examples include acetylthiooxy [i.e., CH₃C(═S)O—], propionylthiooxy, iso-butanoylthiooxy and the like.

The term “C₂₋₄ alkenyl” denotes a radical containing 2 to 4 carbons wherein at least one carbon-carbon double bond is present, some embodiments have 3 carbons, and some embodiments have 2 carbons. Both E and Z isomers and mixtures of E and Z isomers are embraced by the term “alkenyl.” Examples of an alkenyl include vinyl, allyl, 2-butenyl, 3-butenyl, and the like.

The term “C₁₋₄ alkoxy” as used herein denotes a radical alkyl, as defined herein, attached directly to an oxygen atom. Examples include methoxy, ethoxy, n-propoxy, iso-propoxy, n-butoxy, t-butoxy, iso-butoxy and the like.

The term “C₁₋₈ alkyl” and “C₁₋₄ alkyl” denote a straight or branched carbon radical containing 1 to 8 carbons or 1 to 4 carbons respectively, some embodiments are 1 to 6 carbons, some embodiments are 1 to 3 carbons, and some embodiments are 1 or 2 carbons. Examples of an alkyl include methyl, ethyl, n-propyl, iso-propyl, n-butyl, iso-butyl, t-butyl, sec-butyl, n-pentyl, iso-pentyl, sec-pentyl, neo-pentyl, pent-3-yl, 2-methyl-but-1-yl, 1,2-dimethyl-prop-1-yl, n-hexyl, iso-hexyl, sec-hexyl, neo-hexyl, 1-ethyl-2-methyl-prop-1-yl, 1,2,2-trimethyl-prop-1-yl, 1,1,2-trimethyl-prop-1-yl, 1-ethyl-1-methyl-prop-1-yl, 1,1-dimethyl-but-1-yl, 1,2-dimethyl-but-1-yl, 2,3-dimethyl-but-1-yl, 2,2-dimethyl-but-1-yl, 1,3-dimethyl-but-1-yl, hex-3-yl, 2-methyl-pent-1-yl, 3-methyl-1-yl, and the like.

The term “C₁₋₄ alkylcarboxamido” denotes a single alkyl group attached to an amide, wherein alkyl has the same definition as found herein. The C₁₋₅ alkylcarboxamido may be represented by the following:

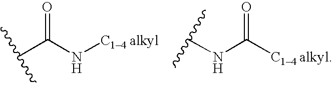

The term “C₁₋₄ alkylsulfinyl” denotes an alkyl radical attached to a sulfoxide radical of the formula: —S(O)— wherein the alkyl radical has the same definition as described herein. Examples include methylsulfinyl, ethylsulfinyl and the like.

The term “C₁₋₄ alkylsulfonamide” refers to the groups

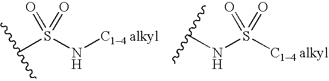

The term “C₁₋₄ alkylsulfonyl” denotes an alkyl radical attached to a sulfone radical of the formula: —S(O)₂— wherein the alkyl radical has the same definition as described herein. Examples include methylsulfonyl, ethylsulfonyl and the like.

The term “C₁₋₄ alkylthio” denotes an alkyl radical attached to a sulfide of the formula: —S— wherein the alkyl radical has the same definition as described herein. Examples include methylsulfanyl (i.e., CH₃S—), ethylsulfanyl, isopropylsulfanyl and the like.

The term “C₁₋₄ alkylamino” denotes one alkyl radical attached to an amino radical wherein the alkyl radical has the same meaning as described herein. Some examples include methylamino, ethylamino, propylamino and the like.

The term “carbo-C₁₋₄-alkoxy” refers to an alkyl ester of a carboxylic acid, wherein the alkyl group is C₁₋₄. Examples include carbomethoxy, carboethoxy, carboisopropoxy and the like.

The term “carboxamide” refers to the group —CONH₂.

The term “cyano” denotes the group CN. The term “C₂₋₆ dialkylamino” denotes an amino substituted with two of the same or different alkyl radicals wherein alkyl radical has the same definition as described herein. Some examples include dimethylamino, methylethylamino, diethylamino and the like.

The term “C₁₋₄ haloalkoxy” denotes a haloalkyl, as defined herein, that is directly attached to an oxygen to form a difluoromethoxy, trifluoromethoxy, 2,2,2-trifluoroethoxy, pentafluoroethoxy and the like.

The term “C₁₋₄ haloalkyl” denotes an alkyl group, defined herein, wherein the alkyl is substituted with at least one halogen up to fully substituted represented by the formula CₙL₂ₙ₊₁, wherein L is a halogen; when more than one halogen is present then they may be the same or different and selected from F, Cl, Br or I. Examples include fluoromethyl, difluoromethyl, trifluoromethyl, chlorodifluoromethyl, 2,2,2-trifluoroethyl, pentafluoroethyl and the like.

The term “C₁₋₄ haloalkylsulfinyl” denotes a haloalkyl radical attached to a sulfoxide of the formula: —S(O)— wherein the alkyl radical has the same definition as described herein. Examples include trifluoromethylsulfinyl, 2,2,2-trifluoroethylsulfinyl, 2,2-difluoroethylsulfinyl and the like.

The term “C₁₋₄ haloalkylsulfonyl” denotes a haloalkyl attached to a sulfone of the formula: —S(O)₂— wherein haloalkyl has the same definition as described herein. Examples include trifluoromethylsulfonyl, 2,2,2-trifluoroethylsulfonyl, 2,2-difluoroethylsulfonyl and the like.

The term “C₁₋₄ haloalkylthio” denotes an alkylthio radical substituted with one or more halogens. Examples include trifluoromethylthio, 1,1-difluoroethylthio, 2,2,2-trifluoroethylthio and the like.

The term “halogen” or “halo” denotes F, Cl, Br and I.

The term “hydroxyl” refers to the group —OH.

The term “thiol” denotes the group —SH.

COMPOSITION shall mean a material comprising at least two compounds or two components; for example, and not limitation, a Pharmaceutical Composition is a Composition.

CONTACT or CONTACTING shall mean bringing the indicated moieties together, whether in an in vitro system or an in vivo system. Thus, “contacting” a 5HT_{2C} receptor with a compound of the invention includes the administration of a compound of the present invention to an individual, preferably a human, having a 5HT_{2C} receptor, as well as, for example, introducing a compound of the invention into a sample containing a cellular or more purified preparation containing a 5HT_{2C} receptor.

IN NEED OF PROPHYLAXIS OR TREATMENT as used herein refers to a judgment made by a caregiver (e.g. physician, nurse, nurse practitioner, etc. in the case of humans; veterinarian in the case of animals, including non-human mammals) that an individual or animal requires or will benefit from prophylaxis or treatment. This judgment is made based on a variety of factors that are in the realm of a caregiver's expertise, but that includes the knowledge that the individual or animal is ill, or will be ill, as the result of a disease, condition or disorder that is treatable by the compounds of the invention. In general, “in need of prophylaxis” refers to the judgment made by the caregiver that the individual will become ill. In this context, the compounds of the invention are used in a protective or preventive manner. However, “in need of treatment” refers to the judgment of the caregiver that the individual is already ill, therefore, the compounds of the present invention are used to alleviate, inhibit or ameliorate the disease, condition or disorder.

INDIVIDUAL as used herein refers to any animal, including mammals, preferably mice, rats, other rodents, rabbits, dogs, cats, swine, cattle, sheep, horses, or primates, and most preferably humans.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

THERAPEUTICALLY EFFECTIVE AMOUNT as used herein refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) Preventing the disease; for example, preventing a disease, condition or disorder in an individual that may be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology or symptomatology of the disease, condition or disorder (i.e., arresting further development of the pathology and/or symptomatology), and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

Compounds of the Invention

One aspect of the present invention pertains to certain substituted N-phenyl-piperazine derivatives as represented by Formula (I):

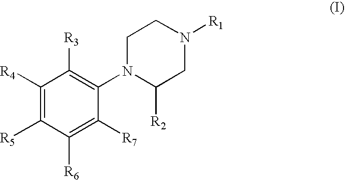

wherein:

R₁ is H or C₁₋₈ alkyl;

R₂ is C₂₋₄ alkenyl, C₁₋₄ alkyl or C₁₋₄ haloalkyl; and

R₃, R₄, R₅, R₆ and R₇ are each independently H, C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl, phenyl, and thiol; or a pharmaceutically acceptable salt, hydrate and solvate thereof.

One aspect of the present invention pertains to certain substituted N-phenyl-piperazine derivatives as represented by Formula (I) wherein:

R₁ is H or C₁₋₈ alkyl;

R₂ is C₂₋₄ alkenyl, C₁₋₄ alkyl or C₁₋₄ haloalkyl; and

R₃, R₄, R₅, R₆ and R₇ are each independently H, C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl and thiol;

or a pharmaceutically acceptable salt, hydrate and solvate thereof.

In some embodiments, the compound is not 1-(4-Chloro-phenyl)-2-methyl-piperazine; 1-(3,5-Difluoro-phenyl)-2-methyl-piperazine; 2-Methyl-1-(2-methylsulfanyl-phenyl)-piperazine; 4-Amino-3-fluoro-2-(2-methyl-piperazin-1-yl)-5-nitro-benzonitrile; 2-Methyl-1-phenyl-piperazine; 4-(2-Isopropyl-piperazin-1-yl)-2-trifluoromethyl-benzonitrile; 4-(2-Ethyl-piperazin-1-yl)-2-trifluoromethyl-benzonitrile; 4-(2-Methyl-piperazin-1-yl)-2-trifluoromethyl-benzonitrile; 1-(3-Chloro-phenyl)-2-methyl-piperazine; 2-Methyl-1-m-tolyl-piperazine; 4-(2-Methyl-piperazin-1-yl)-benzamide; 1-(2-Fluoro-phenyl)-2-methyl-piperazine; 4-(2-Methyl-piperazin-1-yl)-phenol; 1-(3-Methoxy-phenyl)-2-methyl-piperazine; 2-Methyl-1-(3-trifluoromethyl-phenyl)-piperazine; 1-(4-Methoxy-phenyl)-2-methyl-piperazine; 2-Methyl-1-p-tolyl-piperazine; 2,4-Dimethyl-1-phenyl-piperazine; 4-Chloro-5-(4-ethyl-2-methyl-piperazin-1-yl)-benzene-1,2-diamine; 4-Chloro-5-(4-ethyl-2-methyl-piperazin-1-yl)-2-nitro-phenylamine; 5-(4-Ethyl-2-methyl-piperazin-1-yl)-2-nitro-4-trifluoromethyl-phenylamine; and 5-(4-Ethyl-2-methyl-piperazin-1-yl)-4-methyl-2-nitro-phenylamine.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

It is understood and appreciated that compounds of Formula (I) may have one or more chiral centers, and therefore can exist as enantiomers and/or diastereomers. The invention is understood to extend to and embrace all such enantiomers, diastereomers and mixtures thereof, including but not limited to racemates. Accordingly, one embodiment of the present invention pertains to compounds of Formula (I) and formulae used throughout this disclosure that are R enantiomers. Further, one embodiment of the present invention pertains to compounds of Formula (I) and formulae used throughout this disclosure that are S enantiomers. It is understood that compounds of Formula (I) and formulae used throughout this disclosure are intended to represent all individual enantiomers and mixtures thereof, unless stated or shown otherwise.

In some embodiments of the present invention are compounds of Formula (I) wherein R₁ is H. Some embodiments can be represented by Formula (Ia) as illustrated below: 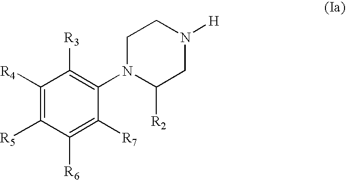
wherein each variable in Formula (Ia) has the same meaning as described herein, supra and infra. In some embodiments, R₂ is methyl. In some embodiments, R₂ is ethyl. In some embodiments, R₂ is a vinyl group (i.e., —CH═CH₂).

Some embodiments of the present invention are compounds of Formula (I) wherein R₁ is C₁₋₈ alkyl.

In some embodiments R₁ is methyl. In some embodiments, compounds can be represented by Formula (Ic) as illustrated below: 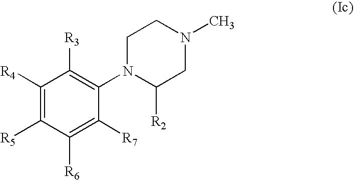
wherein each variable in Formula (Ic) has the same meaning as described herein, supra and infra.

In some embodiments R₁ is ethyl.

In some embodiments R₁ is n-propyl.

In some embodiments R₁ is iso-propyl.

In some embodiments R₁ is n-butyl.

Some embodiments of the present invention are compounds of Formula (I) wherein R₂ is C₂₋₄ alkenyl. In some embodiments R₂ is a vinyl group. In some embodiments, compounds can be represented by Formula (Ie) as illustrated below: 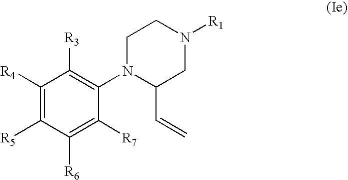
wherein each variable in Formula (Ie) has the same meaning as described herein, supra and infra. In some embodiments, R₁ is H. In still further embodiments, R₁ is CH₃.

Some embodiments of the present invention are compounds of Formula (I) wherein R₂ is C₁₋₄ alkyl. In some embodiments R₂ is methyl. In some embodiments, compounds can be represented by Formula (Ig) as illustrated below: 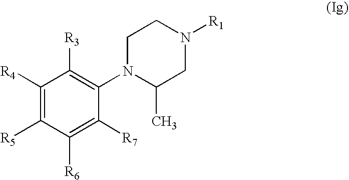
wherein each variable in Formula (Ig) has the same meaning as described herein, supra and infra.

In some embodiments R₂ is ethyl.

In some embodiments R₂ is n-propyl.

Some embodiments of the present invention are compounds of Formula (I) wherein R₂ is C₁₋₄ haloalkyl. In some embodiments R₂ is —CF₃.

Some embodiments of the present invention are compounds of Formula (I) wherein R₃, R₄, R₅, R₆ and R₇ are each independently selected from the group consisting of H, C₁₋₄ alkoxy, C₁₋₄ alkyl, cyano, C₁₋₄ haloalkoxy, C₁₋₄ haloalkyl, halogen, and phenyl.

Some embodiments of the present invention are compounds of Formula (I) wherein R₃, R₄, R₅, R₆ and R₇ are each independently selected from the group consisting of H, C₁₋₄ alkoxy, C₁₋₄ alkyl, cyano, C₁₋₄ haloalkoxy, C₁₋₄ haloalkyl and halogen.

In some embodiments R₃, R₄, R₅, R₆ and R₇ are each independently selected from the group consisting of H, C₁₋₄ alkyl, cyano, C₁₋₄ haloalkoxy, C₁₋₄ haloalkyl and halogen.

In some embodiments R₃, R₄, R₅, R₆ and R₇ are each independently selected from the group consisting of H, C₁₋₄ haloalkoxy, C₁₋₄ haloalkyl and halogen.

In some embodiments R₃, R₄, R₅, R₆ and R₇ are each independently selected from the group consisting of H, CH₃, CH₂CH₃, CH(CH₃)₂, cyano, OCF₃, CF₃, F, Cl, Br, and phenyl.

In some embodiments R₃, R₄, R₅, R₆ and R₇ are each independently selected from the group consisting of H, CH₃, CH₂CH₃, CH(CH₃)₂, cyano, OCF₃, CF₃, F, Cl and Br.

In some embodiments R₃, R₄, R₅, R₆ and R₇ are each independently selected from the group consisting of H, CF₃, F, Cl and Br.

Some embodiments of the present invention are compounds of Formula (I) wherein R₃ is H, CH₃, Br, or F.

Some embodiments of the present invention are compounds of Formula (I) wherein R₃ is H or F.

Some embodiments of the present invention are compounds of Formula (I) wherein R₄ is selected from the group consisting of H, cyano, CH₃, CF₃, F, Cl, Br, phenyl.

Some embodiments of the present invention are compounds of Formula (I) wherein R₄ is selected from the group consisting of H, cyano, F, Cl and Br.

Some embodiments of the present invention are compounds of Formula (I) wherein R₅ is selected from the group consisting of H, CH₃, CH(CH₃)₂, OCF₃, CF₃, F, Cl and Br.

Some embodiments of the present invention are compounds of Formula (I) wherein R₆ is selected from the group consisting of H, CH₃, CF₃, F, Cl and Br.

Some embodiments of the present invention are compounds of Formula (I) wherein R₆ is selected from the group consisting of H, F, Cl and Br.

Some embodiments of the present invention are compounds of Formula (I) wherein R₇ is selected from the group consisting of H, CH₃, F, Cl and Br.

Some embodiments of the present invention are compounds of Formula (I) wherein R₃ is F, R₄ is F or Cl, and R₅, R₆ and R₇ are H. In some embodiments, R₃ is F, R₆ is F or Cl, and R₄, R₅ and R₇ are H. In some embodiments, R₁ is H. In still further embodiments, R₂ is CH₃.

Some embodiments of the present invention are compounds of Formula (I) wherein R₃ is CH₃, R₄ is H or Cl, and R₅, R₆ and R₇ are H. In some embodiments, R₃ is CH₃, R₆ is H or Cl, and R₄, R₅ and R₇ are H. In some embodiments, R₁ is H. In still further embodiments, R₂ is CH₃.

Some embodiments of the present invention are compounds of Formula (I) wherein R₃ is Br, R₅ is H, OCF₃, CF₃ or CH(CH₃)₂, and R₄, R₆ and R₇ are H. In some embodiments, R₁ is H. In still further embodiments, R₂ is CH₃.

Some embodiments of the present invention are compounds of Formula (I) wherein R₃ is Cl, R₅ is H, CH₃ or Cl, and R₄, R₆ and R₇ are H. In some embodiments, R₃ is Cl, R₅ is CH₃ or Cl, and R₄, R₆ and R₇ are H. In some embodiments, R₃is Cl, R₆ is H or Cl, and R₄, R₅ and R₇ are H. In some embodiments, R₃ is Cl, R₆ is Cl, and R₄, R₅ and R₇ are H. In some embodiments, R₁ is H. In still further embodiments, R₂ is CH₃.

Some embodiments of the present invention are compounds of Formula (I) wherein R₄ is Cl, Br or CN, R₅ is H, F or Cl, and R₃, R₆ and R₇ are H. In some embodiments, R₄ is Cl, Br or CN, R₅ is F or Cl, and R₃, R₆ and R₇ are H. In some embodiments, R₄ is Cl, Br or CN, R₆ is H, F or Cl, and R₃, R₅ and R₇ are H. In some embodiments, R₄ is Cl, Br or CN, R₆ is F or Cl, and R₃, R₅ and R₇ are H. In some embodiments, R₁ is H. In still further embodiments, R₂ is CH₃.

Still further embodiments of the present invention are compounds of Formula (I) as shown in the TABLE 2 below.

TABLE 2

| Cmpd # | Structure | Chemical Name |
|---|---|---|
| 1 | 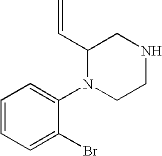 | 1-(2-Bromo-phenyl)-2-vinyl- piperazine |
| 2 | 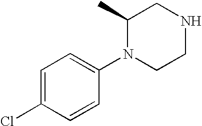 | (R)-1-(4-Chloro-phenyl)-2-methyl- piperazine |
| 3 | 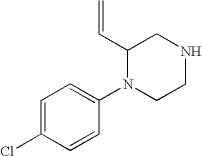 | 1-(4-Chloro-phenyl)-2-vinyl- piperazine |
| 4 | 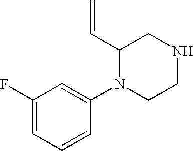 | 1-(3-Fluoro-phenyl)-2-vinyl- piperazine |
| 5 | 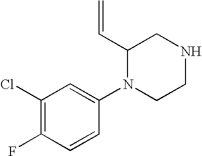 | 1-(3-Chloro-4-fluoro-phenyl)-2- vinyl-piperazine |
| 6 | 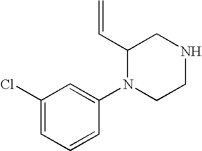 | 1-(3-Chloro-phenyl)-2-vinyl- piperazine |
| 7 | 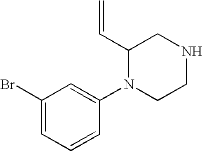 | 1-(3-Bromo-phenyl)-2-vinyl- piperazine |
| 8 | 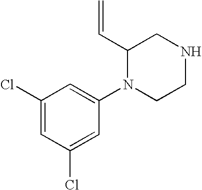 | 1-(3,5-Dichloro-phenyl)-2-vinyl- piperazine |
| 9 | 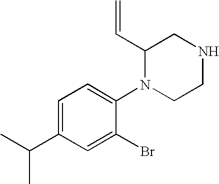 | 1-(2-Bromo-4-isopropyl-phenyl)-2- vinyl-piperazine |
| 10 | 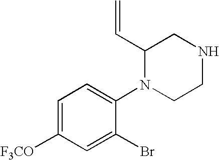 | 1-(2-Bromo-4-trifluoromethoxy- phenyl)-2-vinyl-piperazine |
| 11 | 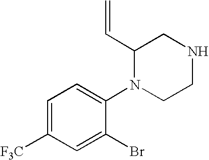 | 1-(2-Bromo-4-trifluoromethyl- phenyl)-2-vinyl-piperazine |
| 12 | 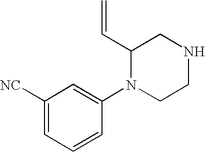 | 3-(2-Vinyl-piperazin-1-yl)- benzonitrile |
| 13 | 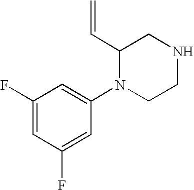 | 1-(3,5-difluoro-phenyl)-2-vinyl- piperazine |
| 14 | 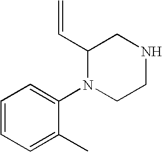 | 1-o-Tolyl-2-vinyl-piperazine |
| 15 | 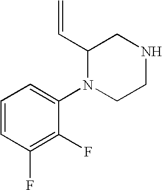 | 1-(2,3-difluoro-phenyl)-2-vinyl- piperazine |
| 16 | 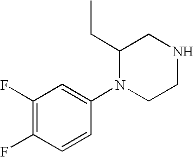 | 1-(2,3-Difluoro-phenyl)-2-ethyl- piperazine |
| 17 | 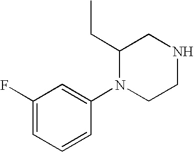 | 1-(3-Fluoro-phenyl)-2-ethyl- piperazine |
| 18 | 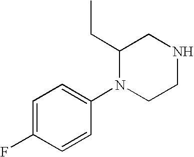 | 1-(4-Fluoro-phenyl)-2-ethyl- piperazine |
| 19 | 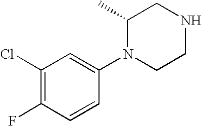 | (R)-1-(3-Chloro-4-fluoro-phenyl)-2- methyl-piperazine |
| 20 | 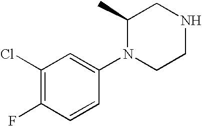 | (S)-1-(3-Chloro-4-fluoro-phenyl)-2- methyl-piperazine |
| 21 | 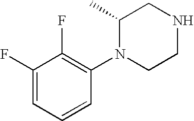 | (R)-1-(3,4-Difluoro-phenyl)-2- methyl-piperazine |
| 22 | 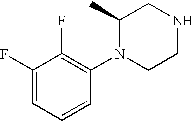 | (S)-1-(3,4-Difluoro-phenyl)-2- methyl-piperazine |
| 23 | 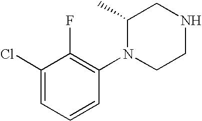 | (R)-1-(3-Chloro-2-fluoro-phenyl)-2- methyl-piperazine |
| 24 | 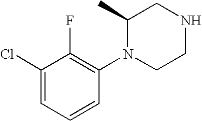 | (S)-1-(3-Chloro-2-fluoro-phenyl)-2- methyl-piperazine |
| 25 | 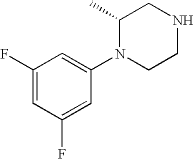 | (R)-1-(3,5-Difluoro-phenyl)-2- methyl-piperazine |
| 26 | 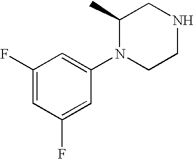 | (S)-1-(3,5-Difluoro-phenyl)-2- methyl-piperazine |
| 27 | 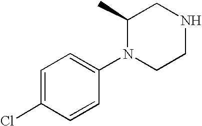 | (S)-1-(4-Chloro-phenyl)-2-methyl- piperazine |
| 28 | 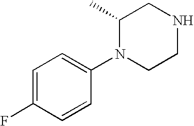 | (R)-1-(4-Fluoro-phenyl)-2-methyl- piperazine |
| 29 | 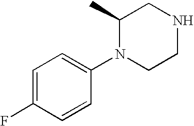 | (S)-1-(4-Fluoro-phenyl)-2-methyl- piperazine |
| 30 | 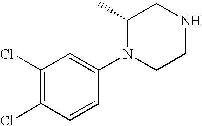 | (R)-1-(3,4-Dichloro-phenyl)-2- methyl-piperazine |
| 31 | 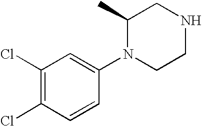 | (S)-1-(3,4-Dichloro-phenyl)-2- methyl-piperazine |
| 32 | 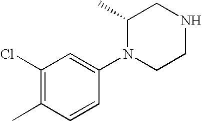 | (R)-1-(3-Chloro-4-methyl-phenyl)- 2-methyl-piperazine |
| 33 | 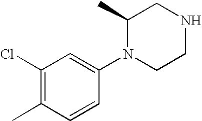 | (S)-1-(3-Chloro-4-methyl-phenyl)-2- methyl-piperazine |
| 34 | 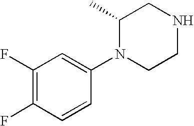 | (R)-1-(3,4-Difluoro-phenyl)-2- methyl-piperazine |
| 35 | 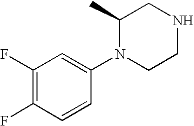 | (S)-1-(3,4-Difluoro-phenyl)-2- methyl-piperazine |
| 36 | 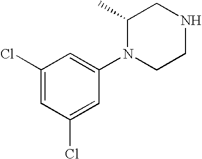 | (R)-1-(3,5-Dichloro-phenyl)-2- methyl-piperazine |
| 37 | 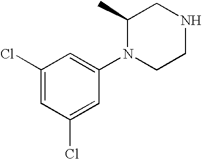 | (S)-1-(3,5-Dichloro-phenyl)-2- methyl-piperazine |
| 38 | 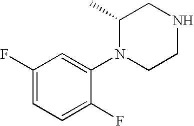 | (R)-1-(2,5-Difluoro-phenyl)-2- methyl-piperazine |
| 39 | 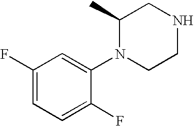 | (S)-1-(2,5-Difluoro-phenyl)-2- methyl-piperazine |
| 40 | 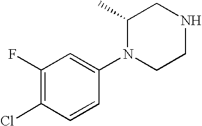 | (R)-1-(4-Chloro-3-fluoro-phenyl)-2- methyl-piperazine |
| 41 | 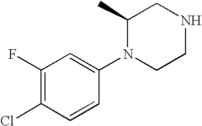 | (S)-1-(4-Chloro-3-fluoro-phenyl)-2- methyl-piperazine |
| 42 | 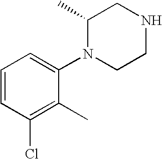 | (R)-1-(3-Chloro-2-methyl-phenyl)- 2-methyl-piperazine |
| 43 | 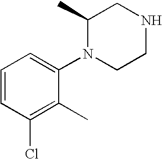 | (S)-1-(3-Chloro-2-methyl-phenyl)-2- methyl-piperazine |
| 44 | 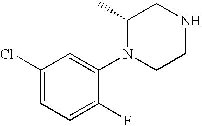 | (R)-1-(5-Chloro-2-fluoro-phenyl)-2- methyl-piperazine |
| 45 | 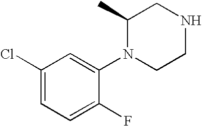 | (S)-1-(5-Chloro-2-fluoro-phenyl)-2- methyl-piperazine |
| 46 | 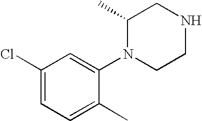 | (R)-1-(5-Chloro-2-methyl-phenyl)- 2-methyl-piperazine |
| 47 | 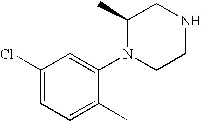 | (S)-1-(5-Chloro-2-methyl-phenyl)-2- methyl-piperazine |
| 48 | 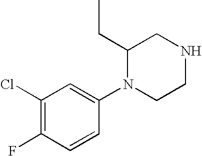 | 1-(3-Chloro-4-fluoro-phenyl)-2- ethyl-piperazine |
| 49 | 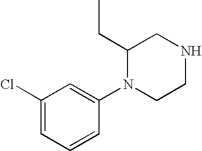 | 1-(3-Chloro-phenyl)-2-ethyl- piperazine |
| 50 | 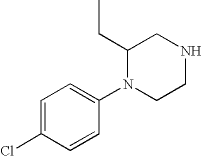 | 1-(4-Chloro-phenyl)-2-ethyl- piperazine |
| 51 | 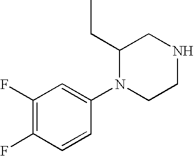 | 1-(3,4-Difluoro-phenyl)-2-ethyl- piperazine |
| 52 | 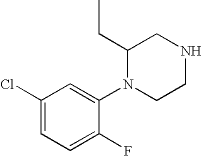 | 1-(5-Chloro-2-fluoro-phenyl)-2- ethyl-piperazine |
| 53 | 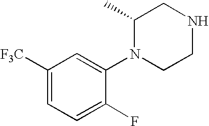 | (R)-1-(2-Fluoro-5-trifluoromethyl- phenyl)-2-methyl-piperazine |
| 54 | 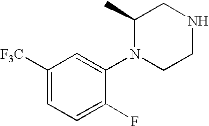 | (S)-1-(2-Fluoro-5-trifluoromethyl- phenyl)-2-methyl-piperazine |
| 55 | 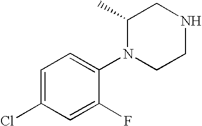 | (R)-1-(4-Chloro-2-fluoro-phenyl)-2- methyl-piperazine |
| 56 | 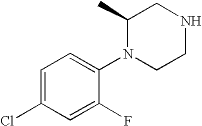 | (S)-1-(4-Chloro-2-fluoro-phenyl)-2- methyl-piperazine |
| 57 | 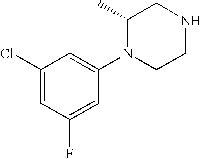 | (R)-1-(3-Chloro-5-fluoro-phenyl)-2- methyl-piperazine |
| 58 | 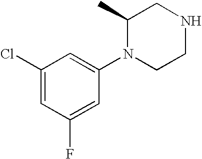 | (S)-1-(3-Chloro-5-fluoro-phenyl)-2- methyl-piperazine |
| 59 | 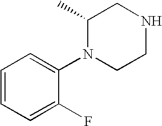 | (R)-1-(2-Fluoro-phenyl)-2-methyl- piperazine |
| 60 | 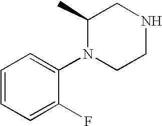 | (S)-1-(2-Fluoro-phenyl)-2-methyl- piperazine |
| 61 | 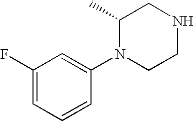 | (R)-1-(3-Fluoro-phenyl)-2-methyl- piperazine |
| 62 | 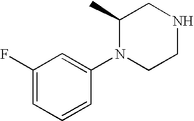 | (S)-1-(3-Fluoro-phenyl)-2-methyl- piperazine |
| 63 | 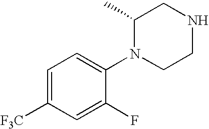 | (R)-1-(2-Fluoro-4-trifluoromethyl- phenyl)-2-methyl-piperazine |
| 64 | 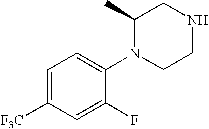 | (S)-1-(2-Fluoro-4-trifluoromethyl- phenyl)-2-methyl-piperazine |
| 65 | 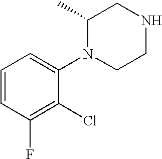 | (R)-1-(2-Chloro-3-fluoro-phenyl)-2- methyl-piperazine |
| 66 | 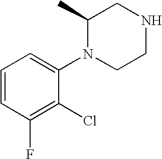 | (S)-1-(2-Chloro-3-fluoro-phenyl)-2- methyl-piperazine |
| 67 | 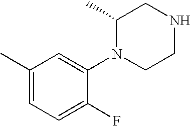 | (R)-1-(2-Fluoro-5-methyl-phenyl)-2- methyl-piperazine |
| 68 | 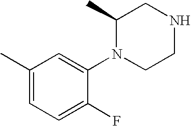 | (S)-1-(2-Fluoro-5-methyl-phenyl)-2- methyl-piperazine |
| 69 | 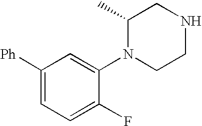 | (R)-1-(4-Fluoro-biphenyl-3-yl)-2- methyl-piperazine |
| 70 | 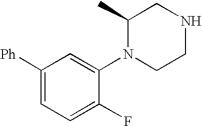 | (S)-1-(4-Fluoro-biphenyl-3-yl)-2- methyl-piperazine |
| 71 | 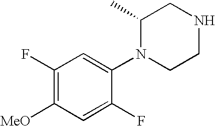 | (R)-1-(2,5-Difluoro-4-methoxy- phenyl)-2-methyl-piperazine |
| 72 | 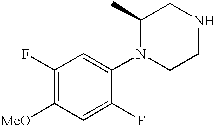 | (S)-1-(2,5-Difluoro-4-methoxy- phenyl)-2-methyl-piperazine |
| 73 | 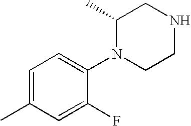 | (R)-1-(2-Fluoro-4-methyl-phenyl)-2- methyl-piperazine |
| 74 | 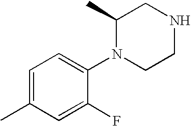 | (S)-1-(2-Fluoro-4-methyl-phenyl)-2- methyl-piperazine |
| 75 | 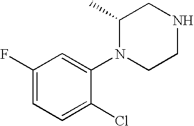 | (R)-1-(2-Chloro-5-fluoro-phenyl)-2- methyl-piperazine |
| 76 | 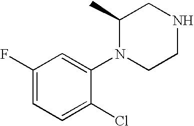 | (S)-1-(2-Chloro-5-fluoro-phenyl)-2- methyl-piperazine |
| 77 | 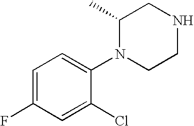 | (R)-1-(2-Chloro-4-fluoro-phenyl)-2- methyl-piperazine |
| 78 | 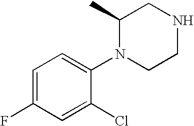 | (S)-1-(2-Chloro-4-fluoro-phenyl)-2- methyl-piperazine |
| 79 | 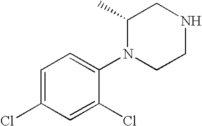 | (R)-1-(2,4-Dichloro-phenyl)-2- methyl-piperazine |
| 80 | 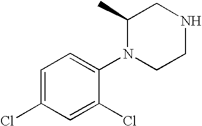 | (S)-1-(2,4-Dichloro-phenyl)-2- methyl-piperazine |
| 81 | 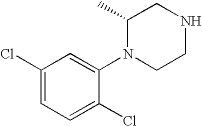 | (R)-1-(2,5-Dichloro-phenyl)-2- methyl-piperazine |
| 82 | 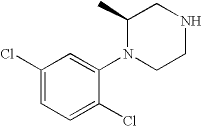 | (S)-1-(2,5-Dichloro-phenyl)-2- methyl-piperazine |
| 83 | 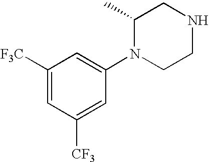 | (R)-1-(3,5-Bis-trifluoromethyl- phenyl)-2-methyl-piperazine |
| 84 | 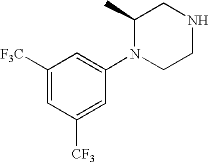 | (S)-1-(3,5-Bis-trifluoromethyl- phenyl)-2-methyl-piperazine |
| 85 | 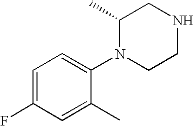 | (R)-1-(4-Fluoro-2-methyl-phenyl)-2- methyl-piperazine |
| 86 | 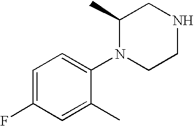 | (S)-1-(4-Fluoro-2-methyl-phenyl)-2- methyl-piperazine |
| 87 | 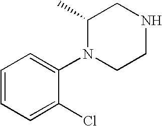 | (R)-1-(2-Chloro-phenyl)-2-methyl- piperazine |
| 88 | 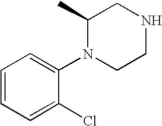 | (S)-1-(2-Chloro-phenyl)-2-methyl- piperazine |
| 89 | 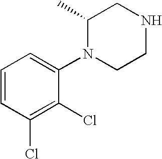 | (R)-1-(2,3-Dichloro-phenyl)-2- methyl-piperazine |
| 90 | 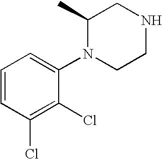 | (S)-1-(2,3-Dichloro-phenyl)-2- methyl-piperazine |
| 91 | 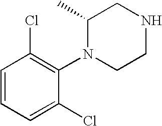 | (R)-1-(2,6-Dichloro-phenyl)-2- methyl-piperazine |
| 92 | 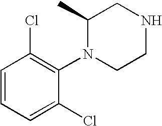 | (S)-1-(2,6-Dichloro-phenyl)-2- methyl-piperazine |
| 93 | 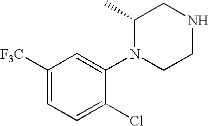 | (R)-1-(2-Chloro-5-trifluoromethyl- phenyl)-2-methyl-piperazine |
| 94 | 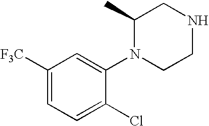 | (S)-1-(2-Chloro-5-trifluoromethyl- phenyl)-2-methyl-piperazine |
| 95 | 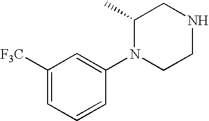 | (R)-2-Methyl-1-(3-trifluoromethyl- phenyl)-piperazine |
| 96 | 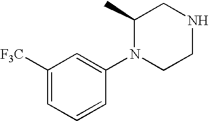 | (S)-2-Methyl-1-(3-trifluoromethyl- phenyl)-piperazine |
| 97 | 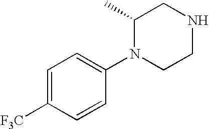 | (R)-2-Methyl-1-(4-trifluoromethyl- phenyl)-piperazine |
| 98 | 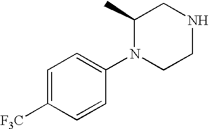 | (S)-2-Methyl-1-(4-trifluoromethyl- phenyl)-piperazine |
| 99 | 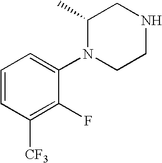 | (R)-1-(2-Fluoro-3-trifluoromethyl- phenyl)-2-methyl-piperazine |
| 100 | 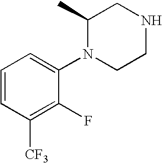 | (S)-1-(2-Fluoro-3-trifluoromethyl- phenyl)-2-methyl-piperazine |
| 101 | 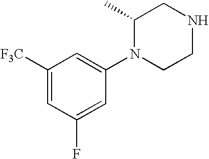 | (R)-1-(3-Fluoro-5-trifluoromethyl- phenyl)-2-methyl-piperazine |
| 102 | 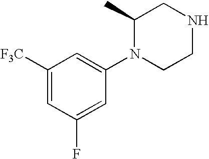 | (S)-1-(3-Fluoro-5-trifluoromethyl- phenyl)-2-methyl-piperazine |
| 103 | 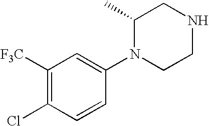 | (R)-1-(4-Chloro-3-trifluoromethyl- phenyl)-2-methyl-piperazine |
| 104 | 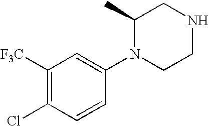 | (S)-1-(4-Chloro-3-trifluoromethyl- phenyl)-2-methyl-piperazine |
| 105 | 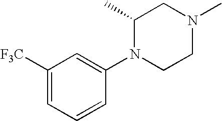 | (R)-2,4-Dimethyl-1-(3- trifluoromethyl-phenyl)-piperazine |
| 106 | 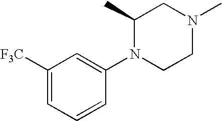 | (S)-2,4-Dimethyl-1-(3- trifluoromethyl-phenyl)-piperazine |
| 107 | 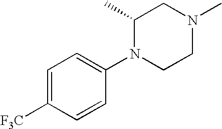 | (R)-2,4-Dimethyl-1-(4- trifluoromethyl-phenyl)-piperazine |
| 108 | 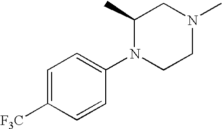 | (S)-2,4-Dimethyl-1-(4- trifluoromethyl-phenyl)-piperazine |
| 109 | 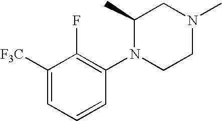 | (S)-1-(2-Fluoro-3-trifluoromethyl- phenyl)-2,4-dimethyl-piperazine |
| 110 | 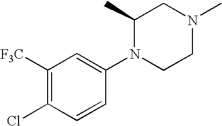 | (S)-1-(4-Chloro-3-trifluoromethyl- phenyl)-2,4-dimethyl-piperazine |
| 111 | 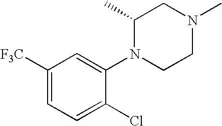 | (R)-1-(2-Chloro-5-trifluoromethyl- phenyl)-2,4-dimethyl-piperazine |
| 112 | 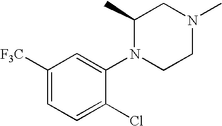 | (S)-1-(2-Chloro-5-trifluoromethyl- phenyl)-2,4-dimethyl-piperazine |
| 113 | 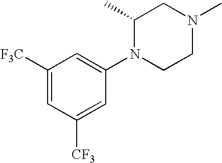 | (R)-1-(3,5-Bis-trifluoromethyl- phenyl)-2,4-dimethyl-piperazine |
| 114 | 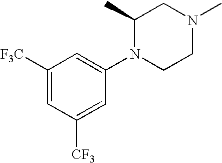 | (S)-1-(3,5-Bis-trifluoromethyl- phenyl)-2,4-dimethyl-piperazine |
| 115 | 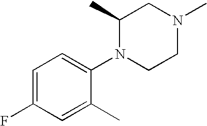 | (S)-1-(4-Fluoro-2-methyl-phenyl)- 2,4-dimethyl-piperazine |
| 116 | 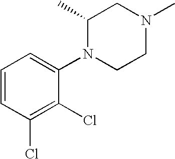 | (R)-1-(2,3-Dichloro-phenyl)-2,4- dimethyl-piperazine |
| 117 | 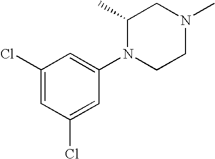 | (R)-1-(3,5-Dichloro-phenyl)-2,4- dimethyl-piperazine |
| 118 | 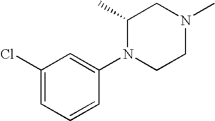 | (R)-1-(3-Chloro-phenyl)-2,4- dimethyl-piperazine |
| 119 | 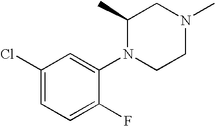 | (R)-1-(5-Chloro-2-fluoro-phenyl)- 2,4-dimethyl-piperazine |
| 120 | 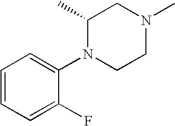 | (S)-1-(2-Fluoro-phenyl)-2,4- dimethyl-piperazine |
| 121 | 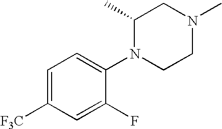 | (S)-1-(2-Fluoro-4-trifluoromethyl- phenyl)-2,4-dimethyl-piperazine |
| 122 | 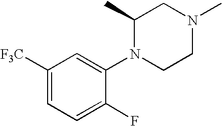 | (S)-1-(2-Fluoro-5-trifluoromethyl- phenyl)-2,4-dimethyl-piperazine |

At various places in the present specification substituents present as a part of the compounds of the invention are disclosed in groups or in ranges. It is specifically intended that the invention include each and every individual subcombination of the members of such groups and ranges. For example, the term “C₁₋₄ alkyl” is specifically intended to individually and separately disclose methyl, ethyl, C₃ alkyl and C₄ allyl.

Some embodiments of the present invention are compounds of Formula (I) selected from the group consisting of: 1-(2-Bromo-phenyl)-2-vinyl-piperazine; 1-(4-Chloro-phenyl)-2-vinyl-piperazine; 1-(3-Fluoro-phenyl)-2-vinyl-piperazine; 1-(3-Chloro-4-fluoro-phenyl)-2-vinyl-piperazine; 1-(3-Chloro-phenyl)-2-vinyl-piperazine; 1-(3-Bromo-phenyl)-2-vinyl-piperazine; 1-(3,5-Dichloro-phenyl)-2-vinyl-piperazine; 1-(2-Bromo-4-isopropyl-phenyl)-2-vinyl-piperazine;1-(2-Bromo-4-trifluoromethoxy-phenyl)-2-vinyl-piperazine; 1-(2-Bromo-4-trifluoromethyl-phenyl)-2-vinyl-piperazine; 3-(2-Vinyl-piperazin-1-yl)-benzonitrile; 1-(3,5-difluoro-phenyl)-2-vinyl-piperazine; 1-o-Tolyl-2-vinyl-piperazine; and 1-(2,3-difluoro-phenyl)-2-vinyl-piperazine; or a pharmaceutically acceptable salt, hydrate and solvate thereof.

Some embodiments of the present invention are compounds of Formula (I) selected from the group consisting of: (R)-1-(2-Bromo-phenyl)-2-vinyl-piperazine; (R)-1-(4-Chloro-phenyl)-2-vinyl-piperazine; (R)-1-(3-Fluoro-phenyl)-2-vinyl-piperazine; (R)-1-(3-Chloro-4-fluoro-phenyl)-2-vinyl-piperazine; (R)-1-(3-Chloro-phenyl)-2-vinyl-piperazine; (R)-1-(3-Bromo-phenyl)-2-vinyl-piperazine; (R)-1-(3,5-Dichloro-phenyl)-2-vinyl-piperazine; (R)-1-(2-Bromo-4-isopropyl-phenyl)-2-vinyl-piperazine; (R)-1-(2-Bromo-4-trifluoromethoxy-phenyl)-2-vinyl-piperazine; (R)-1-(2-Bromo-4-trifluoromethyl-phenyl)-2-vinyl-piperazine; (R)-3-(2-Vinyl-piperazin-1-yl)-benzonitrile; (R)-1-(3,5-difluoro-phenyl)-2-vinyl-piperazine; (R)-1-o-Tolyl-2-vinyl-piperazine; and (R)-1-(2,3-difluoro-phenyl)-2-vinyl-piperazine; or a pharmaceutically acceptable salt, hydrate and solvate thereof.

Some embodiments of the present invention are compounds of Formula (I) selected from the group consisting of: (S)-1-(2-Bromo-phenyl)-2-vinyl-piperazine; (S)-1-(4-Chloro-phenyl)-2-vinyl-piperazine; (S)-1-(3-Fluoro-phenyl)-2-vinyl-piperazine; (S)-1-(3-Chloro-4-fluoro-phenyl)-2-vinyl-piperazine; (S)-1-(3-Chloro-phenyl)-2-vinyl-piperazine; (S)-1-(3-Bromo-phenyl)-2-vinyl-piperazine; (S)-1-(3,5-Dichloro-phenyl)-2-vinyl-piperazine; (S)-1-(2-Bromo-4-isopropyl-phenyl)-2-vinyl-piperazine; (S)-1-(2-Bromo-4-trifluoromethoxy-phenyl)-2-vinyl-piperazine; (S)-1-(2-Bromo-4-trifluoromethyl-phenyl)-2-vinyl-piperazine; (S)-3-(2-Vinyl-piperazin-1-yl)-benzonitrile; (S)-1-(3,5-difluoro-phenyl)-2-vinyl-piperazine; (S)-1-o-Tolyl-2-vinyl-piperazine; and (S)-1-(2,3-difluoro-phenyl)-2-vinyl-piperazine; or a pharmaceutically acceptable salt, hydrate and solvate thereof.

Some embodiments of the present invention are compounds of Formula (I) selected from the group consisting of: 1-(2,3-Difluoro-phenyl)-2-ethyl-piperazine; 1-(3-Fluoro-phenyl)-2-ethyl-piperazine; 1-(4-Fluoro-phenyl)-2-ethyl-piperazine; 1-(3-Chloro-4-fluoro-phenyl)-2-ethyl-piperazine; 1-(3-Chloro-phenyl)-2-ethyl-piperazine; 1-(4-Chloro-phenyl)-2-ethyl-piperazine; 1-(3,4-Difluoro-phenyl)-2-ethyl-piperazine; and 1-(5-Chloro-2-fluoro-phenyl)-2-ethyl-piperazine; or a pharmaceutically acceptable salt, hydrate and solvate thereof.

Some embodiments of the present invention are compounds of Formula (I) selected from the group consisting of: (R)-1-(2,3-Difluoro-phenyl)-2-ethyl-piperazine; (R)-1-(3-Fluoro-phenyl)-2-ethyl-piperazine; (R)-1-(4-Fluoro-phenyl)-2-ethyl-piperazine; (R)-1-(3-Chloro-4-fluoro-phenyl)-2-ethyl-piperazine; (R)-1-(3-Chloro-phenyl)-2-ethyl-piperazine; (R)-1-(4-Chloro-phenyl)-2-ethyl-piperazine; (R)-1-(3,4-Difluoro-phenyl)-2-ethyl-piperazine; and (R)-1-(5-Chloro-2-fluoro-phenyl)-2-ethyl-piperazine; or a pharmaceutically acceptable salt, hydrate and solvate thereof.

Some embodiments of the present invention are compounds of Formula (I) selected from the group consisting of: (S)-1-(2,3-Difluoro-phenyl)-2-ethyl-piperazine; (S)-1-(3-Fluoro-phenyl)-2-ethyl-piperazine; (S)-1-(4-Fluoro-phenyl)-2-ethyl-piperazine; (S)-1-(3-Chloro-4-fluoro-phenyl)-2-ethyl-piperazine; (S)-1-(3-Chloro-phenyl)-2-ethyl-piperazine; (S)-1-(4-Chloro-phenyl)-2-ethyl-piperazine; (S)-1-(3,4-Difluoro-phenyl)-2-ethyl-piperazine; and (S)-1-(5-Chloro-2-fluoro-phenyl)-2-ethyl-piperazine; or a pharmaceutically acceptable salt, hydrate and solvate thereof.

Some embodiments of the present invention are compounds of Formula (I) selected from the group consisting of: 1-(3-Chloro-4-fluoro-phenyl)-2-methyl-piperazine; 1-(3,4-Difluoro-phenyl)-2-methyl-piperazine; 1-(3-Chloro-2-fluoro-phenyl)-2-methyl-piperazine; 1-(4-Fluoro-phenyl)-2-methyl-piperazine; 1-(3,4-Dichloro-phenyl)-2-methyl-piperazine; 1-(3-Chloro-4-methyl-phenyl)-2-methyl-piperazine; 1-(3,4-Difluoro-phenyl)-2-methyl-piperazine; 1-(3,5-Dichloro-phenyl)-2-methyl-piperazine; 1-(2,5-Difluoro-phenyl)-2-methyl-piperazine; 1-(4-Chloro-3-fluoro-phenyl)-2-methyl-piperazine; 1-(3-Chloro-2-methyl-phenyl)-2-methyl-piperazine; 1-(5-Chloro-2-fluoro-phenyl)-2-methyl-piperazine; 1-(5-Chloro-2-methyl-phenyl)-2-methyl-piperazine; 1-(2-Fluoro-5-trifluoromethyl-phenyl)-2-methyl-piperazine; 1-(4-Chloro-2-fluoro-phenyl)-2-methyl-piperazine; 1-(3-Chloro-5-fluoro-phenyl)-2-methyl-piperazine; 1-(3-Fluoro-phenyl)-2-methyl-piperazine; 1-(2-Fluoro-4-trifluoromethyl-phenyl)-2-methyl-piperazine; 1-(2-Chloro-3-fluoro-phenyl)-2-methyl-piperazine; 1-(2-Fluoro-5-methyl-phenyl)-2-methyl-piperazine; 1-(4-Fluoro-biphenyl-3-yl)-2-methyl-piperazine; 1-(2,5-Difluoro-4-methoxy-phenyl)-2-methyl-piperazine; 1-(2-Fluoro-4-methyl-phenyl)-2-methyl-piperazine; 1-(2-Chloro-5-fluoro-phenyl)-2-methyl-piperazine; 1-(2-Chloro-4-fluoro-phenyl)-2-methyl-piperazine; 1-(2,4-Dichloro-phenyl)-2-methyl-piperazine; 1-(2,5-Dichloro-phenyl)-2-methyl-piperazine; 1-(3,5-Bis-trifluoromethyl-phenyl)-2-methyl-piperazine; 1-(4-Fluoro-2-methyl-phenyl)-2-methyl-piperazine; 1-(2-Chloro-phenyl)-2-methyl-piperazine; 1-(2,3-Dichloro-phenyl)-2-methyl-piperazine; 1-(2,6-Dichloro-phenyl)-2-methyl-piperazine; 1-(2-Chloro-5-trifluoromethyl-phenyl)-2-methyl-piperazine; 2-Methyl-1-(4-trifluoromethyl-phenyl)-piperazine; 1-(2-Fluoro-3-trifluoromethyl-phenyl)-2-methyl-piperazine; 1-(3-Fluoro-5-trifluoromethyl-phenyl)-2-methyl-piperazine; and 1-(4-Chloro-3-trifluoromethyl-phenyl)-2-methyl-piperazine; or a pharmaceutically acceptable salt, hydrate and solvate thereof.

Some embodiments of the present invention are compounds of Formula (I) selected from the group consisting of: (R)-1-(3-Chloro-4-fluoro-phenyl)-2-methyl-piperazine; (R)-1-(3,4-Difluoro-phenyl)-2-methyl-piperazine; (R)-1-(3-Chloro-2-fluoro-phenyl)-2-methyl-piperazine; (R)-1-(4-Fluoro-phenyl)-2-methyl-piperazine; (R)-1-(3,4-Dichloro-phenyl)-2-methyl-piperazine; (R)-1-(3-Chloro-4-methyl-phenyl)-2-methyl-piperazine; (R)-1-(3,4-Difluoro-phenyl)-2-methyl-piperazine; (R)-1-(3,5-Dichloro-phenyl)-2-methyl-piperazine; (R)-1-(2,5-Difluoro-phenyl)-2-methyl-piperazine; (R)-1-(4-Chloro-3-fluoro-phenyl)-2-methyl-piperazine; (R)-1-(3-Chloro-2-methyl-phenyl)-2-methyl-piperazine; (R)-1-(5-Chloro-2-fluoro-phenyl)-2-methyl-piperazine; (R)-1-(5-Chloro-2-methyl-phenyl)-2-methyl-piperazine; (R)-1-(2-Fluoro-5-trifluoromethyl-phenyl)-2-methyl-piperazine; (R)-1-(4-Chloro-2-fluoro-phenyl)-2-methyl-piperazine; (R)-1-(3-Chloro-5-fluoro-phenyl)-2-methyl-piperazine; (R)-1-(3-Fluoro-phenyl)-2-methyl-piperazine; (R)-1-(2-Fluoro-4-trifluoromethyl-phenyl)-2-methyl-piperazine; (R)-1-(2-Chloro-3-fluoro-phenyl)-2-methyl-piperazine; (R)-1-(2-Fluoro-5-methyl-phenyl)-2-methyl-piperazine; (R)-1-(4-Fluoro-biphenyl-3-yl)-2-methyl-piperazine; (R)-1-(2,5-Difluoro-4-methoxy-phenyl)-2-methyl-piperazine; (R)-1-(2-Fluoro-4-methyl-phenyl)-2-methyl-piperazine; (R)-1-(2-Chloro-5-fluoro-phenyl)-2-methyl-piperazine; (R)-1-(2-Chloro-4-fluoro-phenyl)-2-methyl-piperazine; (R)-1-(2,4-Dichloro-phenyl)-2-methyl-piperazine; (R)-1-(2,5-Dichloro-phenyl)-2-methyl-piperazine; (R)-1-(3,5-Bis-trifluoromethyl-phenyl)-2-methyl-piperazine; (R)-1-(4-Fluoro-2-methyl-phenyl)-2-methyl-piperazine; (R)-1-(2-Chloro-phenyl)-2-methyl-piperazine; (R)-1-(2,3-Dichloro-phenyl)-2-methyl-piperazine; (R)-1-(2,6-Dichloro-phenyl)-2-methyl-piperazine; (R)-1-(2-Chloro-5-trifluoromethyl-phenyl)-2-methyl-piperazine; (R)-2-Methyl-1-(4-trifluoromethyl-phenyl)-piperazine; (R)-1-(2-Fluoro-3-trifluoromethyl-phenyl)-2-methyl-piperazine; (R)-1-(3-Fluoro-5-trifluoromethyl-phenyl)-2-methyl-piperazine; and (R)-1-(4-Chloro-3-trifluoromethyl-phenyl)-2-methyl-piperazine; or a pharmaceutically acceptable salt, hydrate and solvate thereof.

Some embodiments of the present invention are compounds of Formula (I) selected from the group consisting of: (S)-1-(3-Chloro-4-fluoro-phenyl)-2-methyl-piperazine; (S)-1-(3,4-Difluoro-phenyl)-2-methyl-piperazine; (S)-1-(3-Chloro-2-fluoro-phenyl)-2-methyl-piperazine; (S)-1-(4-Fluoro-phenyl)-2-methyl-piperazine; (S)-1-(3,4-Dichloro-phenyl)-2-methyl-piperazine; (S)-1-(3-Chloro-4-methyl-phenyl)-2-methyl-piperazine; (S)-1-(3,4-Difluoro-phenyl)-2-methyl-piperazine; (S)-1-(3,5-Dichloro-phenyl)-2-methyl-piperazine; (S)-1-(2,5-Difluoro-phenyl)-2-methyl-piperazine; (S)-1-(4-Chloro-3-fluoro-phenyl)-2-methyl-piperazine; (S)-1-(3-Chloro-2-methyl-phenyl)-2-methyl-piperazine; (S)-1-(5-Chloro-2-fluoro-phenyl)-2-methyl-piperazine; (S)-1-(5-Chloro-2-methyl-phenyl)-2-methyl-piperazine; (S)-1-(2-Fluoro-5-trifluoromethyl-phenyl)-2-methyl-piperazine; (S)-1-(4-Chloro-2-fluoro-phenyl)-2-methyl-piperazine; (S)-1-(3-Chloro-5-fluoro-phenyl)-2-methyl-piperazine; (S)-1-(3-Fluoro-phenyl)-2-methyl-piperazine; (S)-1-(2-Fluoro-4-trifluoromethyl-phenyl)-2-methyl-piperazine; (S)-1-(2-Chloro-3-fluoro-phenyl)-2-methyl-piperazine; (S)-1-(2-Fluoro-5-methyl-phenyl)-2-methyl-piperazine; (S)-1-(4-Fluoro-biphenyl-3-yl)-2-methyl-piperazine; (S)-1-(2,5-Difluoro-4-methoxy-phenyl)-2-methyl-piperazine; (S)-1-(2-Fluoro-4-methyl-phenyl)-2-methyl-piperazine; (S)-1-(2-Chloro-5-fluoro-phenyl)-2-methyl-piperazine; (S)-1-(2-Chloro-4-fluoro-phenyl)-2-methyl-piperazine; (S)-1-(2,4-Dichloro-phenyl)-2-methyl-piperazine; (S)-1-(2,5-Dichloro-phenyl)-2-methyl-piperazine; (S)-1-(3,5-Bis-trifluoromethyl-phenyl)-2-methyl-piperazine; (S)-1-(4-Fluoro-2-methyl-phenyl)-2-methyl-piperazine; (S)-1-(2-Chloro-phenyl)-2-methyl-piperazine; (S)-1-(2,3-Dichloro-phenyl)-2-methyl-piperazine; (S)-1-(2,6-Dichloro-phenyl)-2-methyl-piperazine; (S)-1-(2-Chloro-5-trifluoromethyl-phenyl)-2-methyl-piperazine; (S)-2-Methyl-1-(4-trifluoromethyl-phenyl)-piperazine; (S)-1-(2-Fluoro-3-trifluoromethyl-phenyl)-2-methyl-piperazine; (S)-1-(3-Fluoro-5-trifluoromethyl-phenyl)-2-methyl-piperazine; and (S)-1-(4-Chloro-3-trifluoromethyl-phenyl)-2-methyl-piperazine; or a pharmaceutically acceptable salt, hydrate and solvate thereof.

Some embodiments of the present invention are compounds of Formula (I) selected from the group consisting of: 2,4-Dimethyl-1-(3-trifluoromethyl-phenyl)-piperazine; 2,4-Dimethyl-1-(4-trifluoromethyl-phenyl)-piperazine; 1-(2-Fluoro-3-trifluoromethyl-phenyl)-2,4-dimethyl-piperazine; 1-(4-Chloro-3-trifluoromethyl-phenyl)-2,4-dimethyl-piperazine; 1-(2-Chloro-5-trifluoromethyl-phenyl)-2,4-dimethyl-piperazine; 1-(3,5-Bis-trifluoromethyl-phenyl)-2,4-dimethyl-piperazine; 1-(4-Fluoro-2-methyl-phenyl)-2,4-dimethyl-piperazine; 1-(2,3-Dichloro-phenyl)-2,4-dimethyl-piperazine; 1-(3,5-Dichloro-phenyl)-2,4-dimethyl-piperazine; 1-(3-Chloro-phenyl)-2,4-dimethyl-piperazine; 1-(5-Chloro-2-fluoro-phenyl)-2,4-dimethyl-piperazine; 1-(2-Fluoro-phenyl)-2,4-dimethyl-piperazine; 1-(2-Fluoro-4-trifluoromethyl-phenyl)-2,4-dimethyl-piperazine; and 1-(2-Fluoro-5-trifluoromethyl-phenyl)-2,4-dimethyl-piperazine; or a pharmaceutically acceptable salt, hydrate and solvate thereof.

Some embodiments of the present invention are compounds of Formula (I) selected from the group consisting of: (R)-2,4-Dimethyl-1-(3-trifluoromethyl-phenyl)-piperazine; (R)-2,4-Dimethyl-1-(4-trifluoromethyl-phenyl)-piperazine; (R)-1-(2-Fluoro-3-trifluoromethyl-phenyl)-2,4-dimethyl-piperazine; (R)-1-(4-Chloro-3-trifluoromethyl-phenyl)-2,4-dimethyl-piperazine; (R)-1-(2-Chloro-5-trifluoromethyl-phenyl)-2,4-dimethyl-piperazine; (R)-1-(3,5-Bis-trifluoromethyl-phenyl)-2,4-dimethyl-piperazine; (R)-1-(4-Fluoro-2-methyl-phenyl)-2,4-dimethyl-piperazine; (R)-1-(2,3-Dichloro-phenyl)-2,4-dimethyl-piperazine; (R)-1-(3,5-Dichloro-phenyl)-2,4-dimethyl-piperazine; (R)-1-(3-Chloro-phenyl)-2,4-dimethyl-piperazine; (R)-1-(5-Chloro-2-fluoro-phenyl)-2,4-dimethyl-piperazine; (R)-1-(2-Fluoro-phenyl)-2,4-dimethyl-piperazine; (R)-1-(2-Fluoro-4-trifluoromethyl-phenyl)-2,4-dimethyl-piperazine; and (R)-1-(2-Fluoro-5-trifluoromethyl-phenyl)-2,4-dimethyl-piperazine; or a pharmaceutically acceptable salt, hydrate and solvate thereof.

Some embodiments of the present invention are compounds of Formula (I) selected from the group consisting of: (S)-2,4-Dimethyl-1-(3-trifluoromethyl-phenyl)-piperazine; (S)-2,4-Dimethyl-1-(4-trifluoromethyl-phenyl)-piperazine; (S)-1-(2-Fluoro-3-trifluoromethyl-phenyl)-2,4-dimethyl-piperazine; (S)-1-(4-Chloro-3-trifluoromethyl-phenyl)-2,4-dimethyl-piperazine; (S)-1-(2-Chloro-5-trifluoromethyl-phenyl)-2,4-dimethyl-piperazine; (S)-1-(3,5-Bis-trifluoromethyl-phenyl)-2,4-dimethyl-piperazine; (S)-1-(4-Fluoro-2-methyl-phenyl)-2,4-dimethyl-piperazine; (S)-1-(2,3-Dichloro-phenyl)-2,4-dimethyl-piperazine; (S)-1-(3,5-Dichloro-phenyl)-2,4-dimethyl-piperazine; (S)-1-(3-Chloro-phenyl)-2,4-dimethyl-piperazine; (S)-1-(5-Chloro-2-fluoro-phenyl)-2,4-dimethyl-piperazine; (S)-1-(2-Fluoro-phenyl)-2,4-dimethyl-piperazine; (S)-1-(2-Fluoro-4-trifluoromethyl-phenyl)-2,4-dimethyl-piperazine; and (S)-1-(2-Fluoro-5-trifluoromethyl-phenyl)-2,4-dimethyl-piperazine; or a pharmaceutically acceptable salt, hydrate and solvate thereof.

In some embodiments of the present invention a compound is not one or more of the compounds in Table 3.

TABLE 3

| Structure | Chemical Name |
|---|---|
| 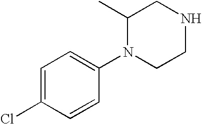 | 1-(4-Chloro-phenyl)- 2-methyl-piperazine |
| 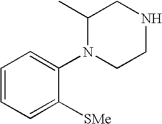 | 2-Methyl-1-(2- methylsulfanyl- phenyl)-piperazine |
| 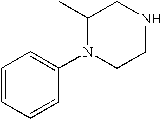 | 2-Methyl-1-phenyl- piperazine |
| 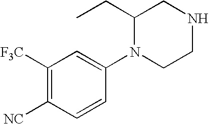 | 4-(2-Ethyl-piperazin- 1-yl)-2- trifluoromethyl- benzonitrile |
| 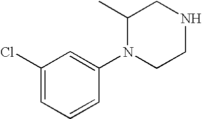 | 1-(3-Chloro-phenyl)- 2-methyl-piperazine |
| 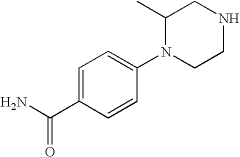 | 4-(2-Methyl-piperazin- 1-yl)-benzamide |
| 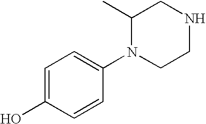 | 4-(2-Methyl-piperazin- 1-yl)-phenol |
| 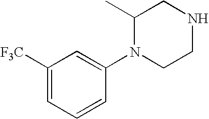 | 2-Methyl-1-(3- trifluoromethyl- phenyl)-piperazine |
| 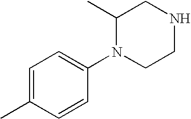 | 2-Methyl-1-p-tolyl- piperazine |
| 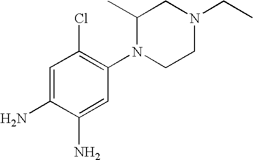 | 4-Chloro-5-(4-ethyl-2- methyl-piperazin-1- yl)-benzene-1,2- diamine |
| 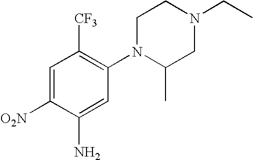 | 5-(4-Ethyl-2-methyl- piperazin-1-yl)-2- nitro-4- trifluoromethyl- phenylamine |
| 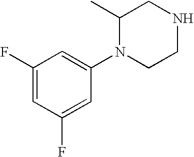 | 1-(3,5-Difluoro- phenyl)-2-methyl- piperazine |
| 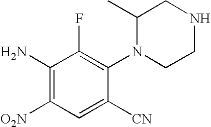 | 4-Amino-3-fluoro-2- (2-methyl-piperazin-1- yl)-5-nitro-benzonitrile |
| 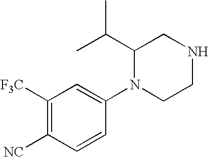 | 4-(2-Isopropyl- piperazin-1-yl)-2- trifluoromethyl- benzonitrile |
| 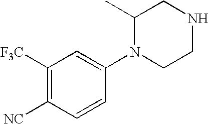 | 4-(2-Methyl-piperazin- 1-yl)-2- trifluoromethyl- benzonitrile |
| 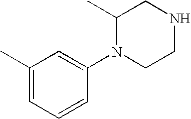 | 2-Methyl-1-m-tolyl- piperazine |
| 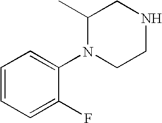 | 1-(2-Fluoro-phenyl)-2- methyl-piperazine |
| 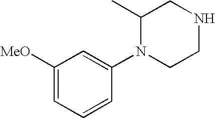 | 1-(3-Methoxy-phenyl)- 2-methyl-piperazine |
| 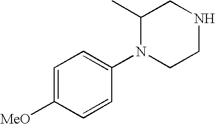 | 1-(4-Methoxy-phenyl)- 2-methyl-piperazine |
| 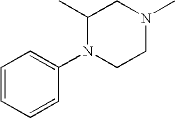 | 2,4-Dimethyl-1- phenyl-piperazine |
| 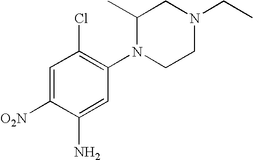 | 4-Chloro-5-(4-ethyl-2- methyl-piperazin-1- yl)-2-nitro- phenylamine |
| 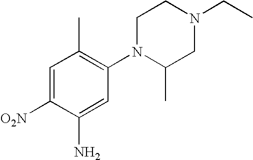 | 5-(4-Ethyl-2-methyl- piperazin-1-yl)-4- methyl-2-nitro- phenylamine |

Methods and Use:

One aspect of the present invention pertains to methods of modulating a 5HT_{2C} receptor comprising contacting said receptor with a therapeutically effective amount or dose of a compound as described herein. Preferably, compounds of the present invention are agonists of the 5HT_{2C} receptor.

Another aspect of the present invention pertains to methods of prophylaxis or treatment of a 5HT_{2C} receptor associated disease in an individual comprising administering to the individual in need of such prophylaxis or treatment a therapeutically effective amount or dose of a compound of the present invention or a pharmaceutical composition thereof. In some embodiments, the 5HT_{2C} receptor associated disease is selected from the group consisting of disorders of the central nervous system; damage to the central nervous system; cardiovascular disorders; gastrointestinal disorders; diabetes insipidus and sleep apnea. In some embodiments, the individual is a mammal. Preferably, the mammal is a human.

In some embodiments, the 5HT_{2C} receptor associated related disease is selected from the group consisting of depression, atypical depression, bipolar disorders, anxiety, anxiety disorders, obsessive-compulsive disorders, social phobias, panic states, attention deficit hyperactivity disorder, disruptive behavior disorders, impulse control disorders, borderline personality disorder, sleep disorders (e.g., sleep apnea), autism, seizure disorders, mutism, selective mutism, childhood anxiety disorders, sexual dysfunction in males (e.g., premature ejaculation and erectile difficulty or dysfunction), sexual dysfunction in females, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, dementia of aging, organic mental disorders, mental disorders in childhood, aggressivity, age-related memory disorders, memory loss, chronic fatigue syndrome, drug and alcohol addiction, alcoholism, tobacco abuse, weight loss, obesity, bulimia, bulimia nervosa, anorexia nervosa, binge eating disorder, premenstrual tension, premenstrual syndrome (PMS or late luteal phase dysphoric disorder), post-traumatic syndrome, spinal cord injury, damage of the central nervous system (e.g., trauma, stroke, neurodegenerative diseases or toxic or infective disorders (e.g., thrombosis), gastrointestinal disorders (e.g., dysfunction of gastrointestinal motility), diabetes insipidus, and type II diabetes.

In some embodiments, the 5HT_{2C} receptor associated disease is selected from the group consisting of high blood pressure, hypertension, high blood cholesterol, dyslipidemia, type II (non-insulin dependent) diabetes, insulin resistance, glucose intolerance, hyperinsulinemia, coronary heart disease, angina pectoris, congestive heart failure, stroke, gallstones, cholescystitis and cholelithiasis, gout, osteoarthritis, obstructive sleep apnea and respiratory problems, some types of cancer (such as endometrial, breast, prostate, and colon), complications of pregnancy, poor female reproductive health (such as menstrual irregularities, infertility, irregular ovulation), bladder control problems (such as stress incontinence), uric acid nephrolithiasis, psychological disorders (such as depression, eating disorders, distorted body image, and low self esteem).

In some embodiments, the 5HT_{2C} receptor associated disease is selected from the group consisting of psychiatric symptoms and behaviors in individuals with eating disorders such as, but not limited to, anorexia nervosa and bulimia nervosa. Individuals with eating disorders often demonstrate social isolation. For example, anorexic individuals often present symptoms of being depressed, anxious, obsession, perfectionistic traits, and rigid cognitive styles as well as sexual disinterest. In addition to anorexia nervosa and bulimia nervosa, other eating disorders include, binge eating disorder (compulsive eating) and ED-NOS (i.e., eating disorders not otherwise specified—an official diagnosis). An individual diagnosed with ED-NOS possess atypical eating disorders including situations in which the individual meets all but a few of the criteria for a particular diagnosis. In essence, what the individual is doing with regard to food and weight is neither normal nor healthy.

In some embodiments, the 5HT_{2C} receptor associated disease is selected from the group consisting of anorexia athletica (compulsive exercising), body dysmorphic disorder (bigorexia), infection-triggered auto immune subtype of anorexia in children, orthorexia nervosa, night-eating syndrome, nocturnal sleep-related eating disorder, rumination syndrome, gourmand syndrome, Prader-Willi syndrome, pica, and cyclic vomiting syndrome.

Another aspect of the present invention pertains to methods of decreasing food intake of an individual comprising administering to the individual a therapeutically effective amount or dose of a compound of the present invention or a pharmaceutical composition thereof. In some embodiments, the individual is a mammal. Preferably, the mammal is a human. In father embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of inducing satiety in an individual comprising administering to said individual a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of controlling weight gain of an individual comprising administering to said individual suffering from weight control a therapeutically effective amount or dose of a compound of the present invention or a pharmaceutical composition thereof. In some embodiments, the individual is a mammal. Preferably, the mammal is a human. In further embodiments, the human has a body mass index of about 18.5 to about 45. In further embodiments, the human has a body mass index of about 25 to about 45. In further embodiments, the human has a body mass index of about 30 to about 45. In further embodiments, the human has a body mass index of about 35 to about 45.

Another aspect of the present invention pertains to methods of producing a pharmaceutical composition comprising admixing at least one compound of the present invention and at least one pharmaceutically acceptable carrier.

Another aspect of the present invention pertains to compounds, as described herein, for use in a method of prophylaxis or treatment of disorders of the central nervous system; damage to the central nervous system; cardiovascular disorders; gastrointestinal disorders; diabetes insipidus or sleep apnea of the human or animal body by therapy.

Another aspect of the present invention pertains to use of compounds, as described herein, for the manufacture of a medicament for use in the treatment or prophylaxis of disorders of the central nervous system; damage to the central nervous system; cardiovascular disorders; gastrointestinal disorders; diabetes insipidus or sleep apnea.

In some embodiments, the disorders of the central nervous system are selected the group consisting of depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension. In further embodiments, the disorder of the central nervous system is obesity. In further embodiments, the disorder of the central nervous system is Alzheimer disease. In further embodiments, the sexual dysfunction is Male erectile dysfunction.

In some embodiments, the damage to the central nervous system is by trauma, stroke, neurodegenerative diseases, toxic CNS diseases or infective CNS diseases. In further embodiments, the damage to the central nervous system is by encephalitis or meningitis.

In some embodiments, the cardiovascular disorder is thrombosis.

In some embodiments, the gastrointestinal disorder is dysfunction of gastrointestinal motility. Another aspect of the present invention pertains to methods of producing a pharmaceutical composition comprising admixing at least one compound of the present invention and at least one pharmaceutically acceptable carrier.

Another aspect of the present invention pertains to compounds, as described herein, for use in a method of treatment of the human or animal body by therapy.

Another aspect of the present invention pertains to compounds, as described herein, for use in a method of prophylaxis or treatment of disorders of the central nervous system; damage to the central nervous system; cardiovascular disorders; gastrointestinal disorders; diabetes insipidus or sleep apnea of the human or animal body by therapy.

Another aspect of the present invention pertains to use of compounds, as described herein, for the manufacture of a medicament for use in the treatment or prophylaxis of disorders of the central nervous system; damage to the central nervous system; cardiovascular disorders; gastrointestinal disorders; diabetes insipidus or sleep apnea.

Another aspect of the present invention pertains to the use of a compound of the present invention with agonist activity at the serotonin 5HT_{2C} receptor for the treatment and/or prophylaxis of AD and AD related disorders. The compounds of the present invention can be used alone or in combination with another agent or agents (such as but not limited to AChE inhibitors) that are typically prescribed for AD.

Combination Therapy—Prophylaxis and Treatment:

In the context of the present invention, a compound of Formula (I) or pharmaceutical composition thereof can be utilized for modulating the activity of the 5HT_{2C} receptor associated diseases, conditions and/or disorders as described herein. Examples of modulating the activity of 5HT_{2C} receptor associated diseases include the prophylaxis or treatment of obesity and/or overweight by decreasing food intake, inducing satiation (i.e., the feeling of fullness), controlling weight gain, decreasing body weight and/or affecting metabolism such that the recipient loses weight and/or maintains weight. Such compounds and pharmaceutical compositions can therefore be used in the context of disorders and/or diseases where weight gain is a component of a disease and/or disorder such as those listed herein. Furthermore, compounds and composition of the present invention can be used for the prophylaxis and/or treatment of Alzheimer Disease, erectile dysfunction and other 5HT_{2C} receptor associated diseases and/or disorders described herein.

While the compounds of the invention can be administered as the sole active pharmaceutical agent (i.e., mono-therapy), they can also be used in combination with other pharmaceutical agents (i.e., combination-therapy) for the treatment of the diseases/conditions/disorders described herein. Therefore, another aspect of the present invention includes methods of prophylaxis and/or treatment comprising administering to an individual in need of prophylaxis and/or treatment a therapeutically effective amount of a compound of the present invention, for example Formula (I), in combination with one or more additional pharmaceutical agent as described herein.

Suitable pharmaceutical agents that can be used in combination with the compounds of the present invention include anti-obesity agents such as apolipoprotein-B secretion/microsomal triglyceride transfer protein (apo-B/MTP) inhibitors, MCR-4 agonists, cholesoystokinin-A (CCK-A) agonists, serotonin and norepinephrine reuptake inhibitors (for example, sibutramine), sympathomimetic agensts, β₃ adrenergic receptor agonists, dopamine agonists (for example, bromocriptine), melanocyte-stimulating hormone receptor analogs, cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide], melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™ available from Regeneron Pharmaceuticals, Inc., Tarrytown, N.Y. and Procter & Gamble Company, Cincinnati, Ohio), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion).

Other anti-obesity agents, including the agents set forth infra, are well known, or will be readily apparent in light of the instant disclosure, to one of ordinary skill in the art.

In some embodiments, the anti-obesity agents are selected from the group consisting of orlistat, sibutramine, bromocriptine, ephedrine, leptin, and pseudoephedrine. In a further embodiment, compounds of the present invention and combination therapies are administered in conjunction with exercise and/or a sensible diet.

It will be understood that the scope of combination-therapy of the compounds of the present invention with other anti-obesity agents, anorectic agents, appetite suppressant and related agents is not limited to those listed above, but includes in principle any combination with any pharmaceutical agent or pharmaceutical composition useful for the treatment of overweight and obese individuals.

Other suitable pharmaceutical agents, in addition to anti-obesity agents, that can be used in combination with the compounds of the present invention include agents useful in the treatment of concomitant diseases. For example, individuals that are over weight or obese increase their risk of morbidity and mortality arising from concomitant diseases, such as, but not limited to, congestive heart failure, type II diabetes, atherosclerosis, dyslipidemia, hyperinsulinemia, hypertension, insulin resistance, hyperglycemia, retinopathy, nephropathy and neuropathy. Treatment for one or more of the diseases cited herein include the use of one or more pharmaceutical agents known in the art belonging to the classes of drugs referred to, but not limited to, the following: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In accordance to one aspect of the present invention, a compound of the present can be used in combination with a pharmaceutical agent or agents belonging to one or more of the classes of drugs cited herein.

It will be understood that the scope of combination-therapy of the compounds of the present invention with other pharmaceutical agents is not limited to those listed herein, supra or infra, but includes in principle any combination with any pharmaceutical agent or pharmaceutical composition useful for the treatment diseases, conditions or disorders that are linked to overweight and obese individuals.

Some embodiments of the present invention include methods of prophylaxis or treatment of a disease, disorder or condition as described herein comprising administering to an individual in need of such prophylaxis or treatment a therapeutically effect amount or dose of a compound of the present invention in combination with at least one pharmaceutical agent selected from the group consisting of: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin. In some embodiments, methods of the present invention include compounds of the present invention and the pharmaceutical agents are administered separately. In further embodiments, compounds of the present invention and the pharmaceutical agents are administered together.

Suitable pharmaceutical agents that can be used in conjunction with compounds of the present invention include α-glucosidase inhibitors. α-Glucosidase inhibitors belong to the class of drugs which competitively inhibit digestive enzymes such as α-amylase, maltase, α-dextrinase, sucrase, etc. in the pancreas and or small intesting. The reversible inhibition by α-glucosidase inhibitors retard, diminish or otherwise reduce blood glucose levels by delaying the digestion of starch and sugars. Some representative examples of α-glucosidase inhibitors include acarbose, N-(1,3-dihydroxy-2-propyl)valiolamine (generic name; voglibose), miglitol, and α-glucosidase inhibitors known in the art.

Suitable pharmaceutical agents that can be used in conjunction with compounds of the present invention include sulfonylureas. The sulfonylureas (SU) are drugs which promote secretion of insulin from pancreatic β cells by transmitting signals of insulin secretion via SU receptors in the cell membranes. Examples of the sulfonylureas include glyburide, glipizide, glimepiride and other sulfonylureas known in the art.

Suitable pharmaceutical agents that can be used in conjunction with compounds of the present invention include the meglitinides. The meglitinides are benzoic acid derivatives represent a novel class of insulin secretagogues. These agents target postprandial hyperglycemia and show comparable efficacy to sulfonylureas in reducing HbA_{1c}. Examples of meglitinides include repaglinide, nateglinide and other meglitinides known in the art.

Suitable pharmaceutical agents that can be used in conjunction with compounds of the present invention include the biguanides. The biguanides represent a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Suitable pharmaceutical agents that can be used in conjunction with compounds of the present invention include the α-glucosidase inhibitors. The α-glucosidase inhibitors competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Examples of α-glucosidase inhibitors include acarbose, N-(1,3-dihydroxy-2-propyl)valiolamine (generic name; voglibose), miglitol, and α-glucosidase inhibitors known in the art.

Suitable pharmaceutical agents that can be used in conjunction with compounds of the present invention include the peroxisome proliferators-activated receptor-γ (i.e., PPAR-γ) agonists. The peroxisome proliferators-activated receptor-γ agonists represent a class of compounds that activates the nuclear receptor PPAR-γ and therefore regulate the transcription of insulin-responsive genes involved in the control of glucose production, transport and utilization. Agents in the class also facilitate the regulation of fatty acid metabolism. Examples of PPAR-γ agonists include rosiglitazone, pioglitazone, tesaglitazar, netoglitazone, GW-409544, GW-501516 and PPAR-γ agonists known in the art.

Suitable pharmaceutical agents that can be used in conjunction with compounds of the present invention include the HMG-CoA reductase inhibitors. The HMG-CoA reductase inhibitors are agents also referred to as Statin compounds that belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. The statins lower serum LDL concentrations by upregulating the activity of LDL receptors and are responsible for clearing LDL from the blood. Some representative examples the statin compounds include rosuvastatin, pravastatin and its sodium salt, simvastatin, lovastatin, atorvastatin, fluvastatin, cerivastatin, rosuvastatin, pitavastatin, BMS's “superstatin”, and HMG-CoA reductase inhibitors known in the art.

Suitable pharmaceutical agents that can be used in conjunction with compounds of the present invention include the angiotensin converting enzyme (ACE) inhibitors. The angiotensin converting enzyme inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the art.

Suitable pharmaceutical agents that can be used in conjunction with compounds of the present invention include the angiotensin II receptor antagonists. Angiotensin II receptor antagonists target the angiotensin II receptor subtype 1 (i.e., AT1) and demonstrate a beneficial effect on hypertension. Examples of angiotensin II receptor antagonists include losartan (and the potassium salt form), and angiotensin II receptor antagonists known in the art.

Other treatments for one or more of the diseases cited herein include the use of pharmaceutical agents known in the art belonging to the classes of drugs referred to, but not limited to, the following: amylin agonists (for example, pramlintide), insulin secretagogues (for example, GLP-1 agonists; exendin-4; insulinotropin (NN2211); dipeptyl peptidase inhibitors (for example, NVP-DPP-728), acyl CoA cholesterol acetyltransferase inhibitors (for example, Ezetimibe, eflucimibe, and like compounds), cholesterol absorption inhibitors (for example, ezetimibe, pamaqueside and like compounds), cholesterol ester transfer protein inhibitors (for example, CP-529414, JTT-705, CETi-1, and like compounds), microsomal triglyceride transfer protein inhibitors (for example, implitapide, and like compounds), cholesterol modulators (for example, NO-1886, and like compounds), bile acid modulators (for example, GT103-279 and like compounds) and squalene synthase inhibitors.

Squalene synthesis inhibitors belong to a class of drugs that lower blood cholesterol levels by inhibiting synthesis of squalene. Examples of the squalene synthesis inhibitors include (S)-α-[Bis[2,2-dimethyl-1-oxopropoxy)methoxy]phosphinyl]-3-phenoxybenzenebutanesulfonic acid, mono potassium salt (BMS-188494) and squalene synthesis inhibitors known in the art.

Compositions of the Present Invention

According to a further aspect, the present invention also pertains to pharmaceutical compositions comprising one or more compounds of Formula (I) or any formulae disclosed herein, and one or more pharmaceutically acceptable carriers.

Some embodiments of the present invention include a method of producing a pharmaceutical composition comprising admixing at least one compound according to any of the compound embodiments disclosed herein and a pharmaceutically acceptable carrier.

Formulations may be prepared by any suitable method, typically by uniformly mixing the active compound(s) with liquids or finely divided solid carriers, or both, in the required proportions, and then, if necessary, forming the resulting mixture into a desired shape.

Conventional excipients, such as binding agents, fillers, acceptable wetting agents, tabletting lubricants, and disintegrants may be used in tablets and capsules for oral administration. Liquid preparations for oral administration may be in the form of solutions, emulsions, aqueous or oily suspensions, and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives, and flavorings and colorants may be added to the liquid preparations. Parenteral dosage forms may be prepared by dissolving the compound of the invention in a suitable liquid vehicle and filter sterilizing the solution before filling and sealing an appropriate vial or ampoule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, editors: Gennaro, A. R., et al.).

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier. Accordingly, another aspect of the present invention pertains to pharmaceutical compositions comprising a pharmaceutical acceptable carrier in combination with at least one compound according to Formula (I):

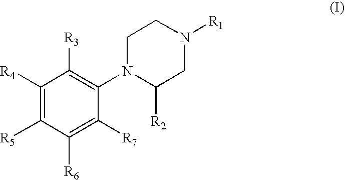

wherein:

R₁ is H or C₁₋₈ alkyl;

R₂ is C₂₋₄ alkenyl, C₁₋₄ alkyl or C₁₋₄ haloalkyl; and

R₃, R₄, R₅, R₆ and R₇ are each independently H, C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl, phenyl, and thiol; or a pharmaceutically acceptable salt, hydrate and solvate thereof.

In some embodiments of each of the genera disclosed in this specification, such as but not limited to, those related to pharmaceutical compositions, the following group of compounds and combinations or subcombinations thereof, are not included therein:

1-(4-Chloro-phenyl)-2-methyl-piperazine; 1-(3,5-Difluoro-phenyl)-2-methyl-piperazine; 2-Methyl-1-(2-methylsulfanyl-phenyl)-piperazine; 4-Amino-3-fluoro-2-(2-methyl-piperazin-1-yl)-5-nitro-benzonitrile; 2-Methyl-1-phenyl-piperazine; 4-(2-Isopropyl-piperazin-1-yl)-2-trifluoromethyl-benzonitrile; 4-(2-Ethyl-piperazin-1-yl)-2-trifluoromethyl-benzonitrile; 4-(2-Methyl-piperazin-1-yl)-2-trifluoromethyl-benzonitrile; 1-(3-Chloro-phenyl)-2-methyl-piperazine; 2-Methyl-1-m-tolyl-piperazine; 4-2-Methyl-piperazin-1-yl)-benzamide; 1-(2-Fluoro-phenyl)-2-methyl-piperazine; 4-(2-Methyl-piperazin-1-yl)-phenol; 1-(3-Methoxy-phenyl)-2-methyl-piperazine; 2-Methyl-1-(3-trifluoromethyl-phenyl)-piperazine; 1-(4-Methoxy-phenyl)-2-methyl-piperazine; 2-Methyl-1-p-tolyl-piperazine; 2,4-Dimethyl-1-phenyl-piperazine; 4-Chloro-5-(4-ethyl-2-methyl-piperazin-1-yl)-benzene-1,2-diamine; 4-Chloro-5-(4-ethyl-2-methyl-piperazin-1-yl)-2-nitro-phenylamine; 5-(4-Ethyl-2-methyl-piperazin-1-yl)-2-nitro-4-trifluoromethyl-phenylamine; and 5-(4-Ethyl-2-methyl-piperazin-1-yl)-4-methyl-2-nitro-phenylamine.

The invention further provides pharmaceutical formulations comprising a compound of the invention or a pharmaceutically acceptable salt or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof, and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate or physiologically functional derivative thereof can be used as active ingredients in pharmaceutical compositions, specifically as 5HT_{2C} receptor agonists. By the term “active ingredient” is defined in the context of a “pharmaceutical composition” and shall mean a component of a pharmaceutical composition that provides the primary pharmacological effect, as opposed to an “inactive ingredient” which would generally be recognized as providing no pharmaceutical benefit.

The dose when using the compounds of the present invention can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but not limited to, about 0.001 mg to about 5000 mg, about 0.001 to about 2500 mg, about 0.001 to about 1000 mg, 0.001 to about 500 mg, 0.001 mg to about 250 mg, about 0.001 mg to 100 mg, about 0.001 mg to about 50 mg, and about 0.001 mg to about 25 mg. Multiple doses may be administered during the day, especially when relatively large amounts are deemed to be needed, for example 2, 3 or 4, doses. Depending on the individual and as deemed appropriate from the patient's physician or care-giver it may be necessary to deviate upward or downward from the doses described herein.

The amount of active ingredient, active salt or hydrate thereof, required for use in treatment will vary not only with the particular salt selected but also with the route of administration, the nature of the condition being treated and the age and condition of the patient and will ultimately be at the discretion of the attendant physician or clinician.

In general, one skilled in the art understands how to extrapolate in vivo data obtained in a model system, typically an animal model, to another, such as a human. Typically, animal models include, but are not limited to, rodent models. In some circumstances, these extrapolations may merely be based on the weight of the animal model in comparison to another, such as a mammal, preferably a human, however, more often, these extrapolations are not simply based on weights, but rather incorporate a variety of factors. Representative factors include, but are not limited to, the type, age, weight, sex, diet and medical condition of the patient, the severity of the disease, the route of administration, pharmacological considerations such as the activity, efficacy, pharmacolinetic and toxicology profiles of the particular compound employed, whether a drug delivery system is utilized, on whether an acute or chronic disease state is being treated or prophylaxis is conducted or on whether further active compounds are administered in addition to the compounds of the Formula (I) as part of combination-therapy. The dosage regimen for treating a disease condition with the compounds and/or compositions of the present invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the case of dropper or pipette, the formulation may be achieved by the patient whereby administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the Formula (I) or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the Formula (I) as an aerosol can be prepared by processes well-known to the person skilled in the art. For their preparation, for example, solutions or dispersions of the compounds of the Formula (I) in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like, such as those pharmaceutically acceptable salts listed in Journal of Pharmaceutical Science, 66, 2 (1977); incorporated herein by reference in its entirety.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In general, the “pro-drug” approach is utilized to facilitate oral absorption. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Some embodiments of the present invention include a method of producing a pharmaceutical composition for “combination-therapy” comprising admixing at least one compound according to any of the compound embodiments disclosed herein, at least one pharmaceutical agent as described herein and a pharmaceutically acceptable carrier.

In some embodiments the pharmaceutical agents is selected from the group consisting of: apolipoprotein-B secretion/microsomal triglyceride transfer protein (apo-B/MTP) inhibitors, MCR-4 agonists, cholescystokinin-A (CCK-A) agonists, serotonin and norepinephrine reuptake inhibitors (for example, sibutramine), sympathomimetic agensts, β₃ adrenergic receptor agonists, dopamine agonists (for example, bromocriptine), melanocyte-stimulating hormone receptor analogs, cannabinoid 1 receptor antagonists [for example, SR141716: N-(piperidin-1-yl)-5-(4-chlorophenyl)-1-(2,4-dichlorophenyl)-4-methyl-1H-pyrazole-3-carboxamide], melanin concentrating hormone antagonists, leptons (the OB protein), leptin analogues, leptin receptor agonists, galanin antagonists, lipase inhibitors (such as tetrahydrolipstatin, i.e., Orlistat), anorectic agents (such as a bombesin agonist), Neuropeptide-Y antagonists, thyromimetic agents, dehydroepiandrosterone or an analogue thereof, glucocorticoid receptor agonists or antagonists, orexin receptor antagonists, urocortin binding protein antagonists, glucagon-like peptide-1 receptor agonists, ciliary neutrotrophic factors (such as Axokine™), human agouti-related proteins (AGRP), ghrelin receptor antagonists, histamine 3 receptor antagonists or reverse agonists, neuromedin U receptor agonists, noradrenergic anorectic agents (for example, phentermine, mazindol and the like) and appetite suppressants (for example, bupropion). In further embodiments, the pharmaceutical agent is selected from the group consisting of orlistat, sibutramine, bromocriptine, ephedrine, leptin, and pseudoephedrine.

In some embodiments the pharmaceutical agents is selected from the group consisting of: sulfonylureas, meglitinides, biguanides, α-glucosidase inhibitors, peroxisome proliferators-activated receptor-γ (i.e., PPAR-γ) agonists, insulin, insulin analogues, HMG-CoA reductase inhibitors, cholesterol-lowering drugs (for example, fibrates that include: fenofibrate, bezafibrate, gemfibrozil, clofibrate and the like; bile acid sequestrants which include: cholestyramine, colestipol and the like; and niacin), antiplatelet agents (for example, aspirin and adenosine diphosphate receptor antagonists that include: clopidogrel, ticlopidine and the like), angiotensin-converting enzyme inhibitors, angiotensin II receptor antagonists and adiponectin.

It is noted that when the 5HT_{2C} receptor agonists are utilized as active ingredients in a pharmaceutical composition, these are not intended for use only in humans, but in other non-human mammals as well. Indeed, recent advances in the area of animal health-care mandate that consideration be given for the use of 5HT_{2C} receptor agonists for the treatment of obesity and related disorders in domestic animals (e.g., cats and dogs), and 5HT_{2C} receptor agonists in other domestic animals where no disease or disorder is evident (e.g., food-oriented animals such as cows, chickens, fish, etc.). Those of ordinary skill in the art are readily credited with understanding the utility of such compounds in such settings.

Preparation of Compounds of the Invention:

In the illustrated syntheses outlined below, the labeled substituents have the same identifications as set out in the definitions of the compounds of the present invention of Formula (I) and the Formulae of the subgenera as described herein.

Those of skill in the art will appreciate the wide variety of compounds of the present invention can be prepared according to Schemes I and II, Infra. One representative synthesis is set forth below in Scheme I:

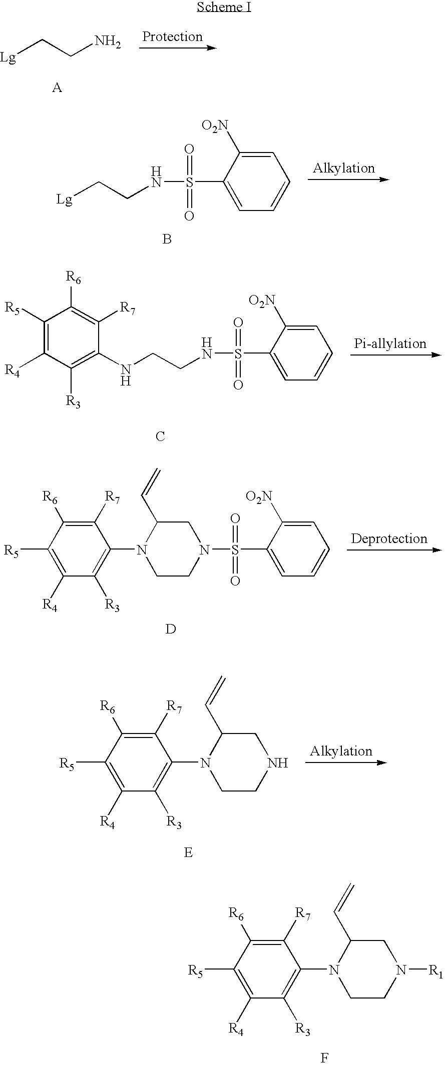

Anilines, either commercially available or prepared via methods known in the art, can be utilized to prepare intermediate C. Accordingly, a wide variety of R₃, R₄, R₅, R₆ and R₇ groups can be introduced. Compounds of the present invention where R₁ is H and R₂ alkenyl (i.e., such as vinyl, Compound E) are prepared via a cyclization of intermediate C followed by deprotection of the amine. In a subsequent step, Compound E can be readily alkylated by, for example, treatment with excess paraformaldehyde (for methylation) or a higher order aldehyde, followed by reduction with NaBH₃CN or similar reducing agent according to methodologies known in the art. Alternatively, Compound E can be readily alkylated, for example, by using an alkyl halide in the presence of abase.

Another representative synthetic pathway for the preparation of compounds of Formula (I) is set forth below in Reaction Scheme II:

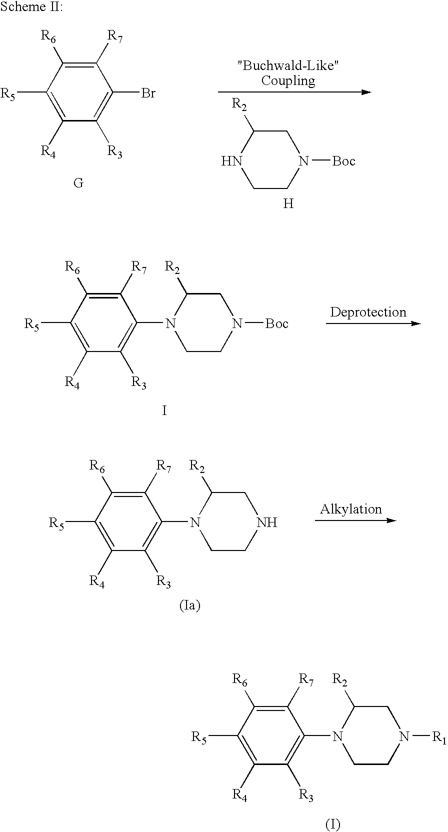

By utilizing, for example, an appropriate substituted aryl-Lg (Compound G wherein Lg is Br as shown Scheme II) and a mono-protected-2-substituted piperazine (Compound H) a wide variety of compounds of the present invention can be prepared.

Protecting groups may be required for various functionality or functionalities during the synthesis of some of the compounds of the invention. Accordingly, representative protecting groups that are suitable for a wide variety of synthetic transformations are disclosed in Greene and Wuts, Protective Groups in Organic Synthesis, 3rd edition, John Wiley & Sons, New York, 1999, the disclosure of which is incorporated herein by reference in its entirety.

As described herein, compounds of the present invention can exist in various forms, for example, enantiomers and racemates. In is understood that the optically active forms can be obtained by resolution of the racemates, separated by chiral chromatography or by asymmetric synthesis using methods known in the art to obtain enantiomers.

Other Utilities

Another object of the present invention relates to radio-labeled compounds of Formula (I) that would be useful not only in radio-imaging but also in assays, both in vitro and in vivo, for localizing and quantitating the 5HT_{2C} receptor in tissue samples, including human, and for identifying 5HT_{2C} receptor ligands by inhibition binding of a radio-labeled compound. It is a further object of this invention to develop novel 5HT_{2C} receptor assays of which comprise such radio-labeled compounds.

The present invention embraces isotopically-labeled compounds of Formula (I) and any subgenera herein, such as but not limited to, Formula (Ia) through Formula (Ig). An “isotopically” or “radio-labeled” compounds are those which are identical to compounds disclosed herein, but for the fact that one or more atoms are replaced or substituted by an atom having an atomic mass or mass number different from the atomic mass or mass number typically found in nature (i.e., naturally occurring). Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. The radionuclide that is incorporated in the instant radio-labeled compounds will depend on the specific application of that radio-labeled compound. For example, for in vitro 5HT_{2C} receptor labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound of Formula (I) that has incorporated at least one radionuclide; in some embodiments the radionuclide is selected from the group consisting of ³H, ¹⁴C, ¹²⁵I, ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Schemes supra and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the more scarce radio-isotope or nonradio-active isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas—This procedure normally yields high specific activity products and requires halogenated or unsaturated precursors.

B. Reduction with Sodium Borohydride [³H]—This procedure is rather inexpensive and requires precursors containing reducible functional groups such as aldehydes, ketones, lactones, esters, and the like.

C. Reduction with Lithium Aluminum Hydride [³H ]—This procedure offers products at almost theoretical specific activities. It also requires precursors containing reducible functional groups such as aldehydes, ketones, lactones, esters, and the like.

D. Tritium Gas Exposure Labeling—This procedure involves exposing precursors containing exchangeable protons to tritium gas in the presence of a suitable catalyst.

E. N-Methylation using Methyl Iodide [³H]—This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions—This procedure transforms an aryl or heteroaryl amine into a diazonium salt; such as a tetrafluoroborate salt, and subsequently to ¹²⁵I labeled compound using Na¹²⁵I. A represented procedure was reported by Zhu, D.-G. and co-workers in J. Org. Chem. 2002, 67, 943-948.

B. Ortho ¹²⁵Iodination of phenols—This procedure allows for the incorporation of ¹²⁵I at the ortho position of a phenol as reported by Collier, T. L. and co-workers in J. Labeled Compd Radiopharm. 1999, 42, S264-S266.

C. Aryl and heteroaryl bromide exchange with ¹²⁵I—This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A represented procedure was reported by Bas, M.-D. and co-workers in J. Labeled Compd Radiopharm. 2001, 44, S280-S282.

A radio-labeled 5HT_{2C} receptor compound of Formula (I) can be used in a screening assay to identify/evaluate compounds. In general terms, a newly synthesized or identified compound (i.e., test compound) can be evaluated for its ability to reduce binding of the “radio-labeled compound of Formula (I)” to the 5HT_{2C} receptor. Accordingly, the ability of a test compound to compete with the “radio-labeled compound of Formula (I)” for the binding to the 5HT_{2C} receptor directly correlates to its binding affinity.

The labeled compounds of the present invention bind to the 5HT_{2C} receptor. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM, and in still yet another embodiment the labeled inhibitor has an IC₅₀ less than about 0.1 μM.

Other uses of the disclosed receptors and methods will become apparent to those in the art based upon, inter alia, a review of this disclosure.

As will be recognized, the steps of the methods of the present invention need not be performed any particular number of times or in any particular sequence. Additional objects, advantages, and novel features of this invention will become apparent to those skilled in the art upon examination of the following examples thereof, which are intended to be illustrative and not intended to be limiting.

### EXAMPLES

### Example 1

### Intracellular IP₃ Accumulation Assay:

HEK293 cells were transfected in 15 cm sterile dishes with or without (control) 16 ug of human 5HT_{2C} receptor cDNA [for example see, Saltzman, A. G., et al. Biochem. Biophys. Res. Commun. 181, 1469-1478 (1991)] using 25 ul of lipofectamine. Cells were then incubated for 3-4 hours at 37° C./5% CO₂ and then transfection media was removed and replaced with 100 ul of DMEM. Cells were then plated onto 100 cm sterile dishes. The next day cells were plated into 96 well PDL microtiter plates at a density of 55K/0.2 ml. Six hours latter, media was exchanged with [³H]inositol (0.25 uCi/well) in inositol free DMEM and plates were incubated at 37° C./5% CO₂ overnight. The next day, wells were aspirated and 200 ul of DMEM containing test compound, 10 uM pargyline, and 10 mM LiCl was added to appropriate wells. Plates were then incubated at 37° C./5% CO₂ for three hours followed aspiration and by addition of fresh ice cold stop solution (1M KOH, 19 mM Na-borate, 3.8 mM EDTA) to each well. Plates were kept on ice for 5-10 min and the wells were neutralized by addition of 200 ul of fresh ice cold neutralization solution (7.5% HCl). Plates were then frozen until further processing is desired. The lysate was then transferred into 1.5 ml Eppendorf tubes and 1 ml of chloroform/methanol (1:2) was added/tube. The solution was vortexed for 15 seconds and the upper phase was applied to a Biorad AG1-X8™ anion exchange resin (100-200 mesh). First, the resin was washed with water at 1:1.25 W/V and 0.9 ml of upper phase was loaded onto the column. The column was then washed with 10 ml of 5 mM myo-inositol and 10 ml of 5 mM Na-borate/60 mM Na-formate. The inositol tris phosphates were eluted into scintillation vials containing 10 ml of scintillation cocktail with 2 ml of 0.1 M formic acid/1 M ammonium formate. The columns were regenerated by washing with 10 ml of 0.1 M formic acid/3M ammonium formate and rinsed twice with dd H₂O and stored at 4° C. in water.

The biological activities in the IP Accumulation Assay for several representative compounds are shown in Table 4 below:

TABLE 4

| | 5HT_{2C} (EC₅₀) |
|---|---|
| Compound No. | IP Accumulation Assay (nM) |
| 23 | 7.4 |
| 44 | 8.0 |

Certain compounds of the present invention are selective for the 5HT_{2C} receptor compared to the 5HT_{2A} and 5HT_{2B} receptors; for example Compound 23 has an EC₅₀ value of 44 nM against the 5HT_{2A} receptor and is essentially inactive against the 5HT_{2B} receptor, and Compound 44 has an EC₅₀ value of 529 nM against the 5HT_{2A} receptor and is essentially inactive against the 5HT_{2B} receptor.

### Example 2

### Inhibition of Basal Food Intake Rats

Male Sprague-Dawley rats (225-325 g) were accustomed to a reverse day/night schedule (lights on 6:30 pm to 10:30 am) for at least 10 days prior to testing. On the test day, the animals were weighed and placed into individual cages (no bedding) at 9:00 am with free access to water. At 10:00 am, animals were injected with test compound or vehicle (2 ml/kg, p.o.), with treatment groups counter-balanced according to animal weights. Immediately upon lights out at 10:30 am, each animal was presented with a pre-weighed amount of food in a dish. Food consumption over different time points was then determined by weighing the food cup at 2, 4, 6 and 22 hrs after the food was presented. Thus, food consumption was measured at 2.5, 4.5, 6.5, and 22.5 hrs post-injection.

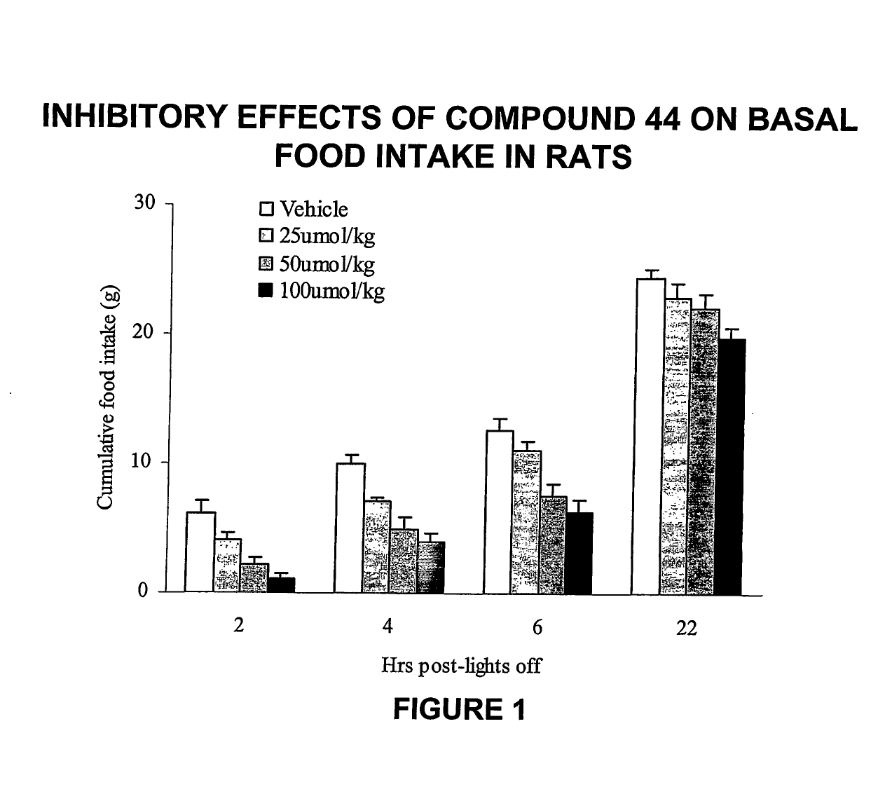

FIG. 1 illustrates the effects of Compound 44 of the present invention on basal food intake in rats. The compound inhibited food intake relative to vehicle-treated controls after administration of all doses over the first 4 hr after food presentation. This effect was maintained to 22 hrs after food presentation at the highest dose tested. ED₅₀s (mol/kg, p.o.) at 2, 4, 6, and 22 after food presentation were 33, 58, 97, and 441, respectively.

### Example 3

### Syntheses of Selected Compounds of the Invention

### Example 3.1

### Preparation of (R,S) 1-(2-Bromo-phenyl)-2-vinyl-piperazine trifluoroacetic acid salt (Compound 1 TFA salt)

### Step 1: Preparation of N-(2-Bromo-ethyl)-2-nitro-benzenesulfonamide

Diisopropylethylamine (20 mL, 115 mmol) was slowly added to an ice-cold solution of bromoethylamine hydrobromide (9.8 g, 48 mmol) and 2-nitrobenzenesulfnyl chloride (10 g, 45 mmol) in 200 mL of methylene chloride. After stirring for one hour in an ice-bath, the crude organic solution was warmed to room temperature and then washed with 1 M HCl (3×75 mL) and saturated aqueous NaHCO₃ (2×75 mL). The resulting organic solution was dried over MgSO₄, vacuum filtered, and concentrated to a yellow solid. Purification by column chromatography on silica gel (EtOAc-hexanes, 1:1) afforded 11.5 g (82%) of a yellow solid. ¹H NMR (400 MHz, CDCl₃) δ 8.17-8.13 (m, 1 H), 7.94-7.90 (m, 1 H), 7.80-7.75 (m, 2 H), 5.87 (t, J=2.8 Hz, 1 H), 3.55 (q, J=6.5 Hz, 2 H), 3.48-3.45 (m, 2 M). MS calculated for C₈H₁₀BrN₂O₄S+H: 309, observed: 309.

### Step 2: Preparation of N-[2-(2-Bromo-phenylamino)-ethyl]-2-nitro-benzenesulfonamide

A solution of N-(2-Bromo-ethyl)-2-nitro-benzenesulfonamide (2.0 g, 6.5 mmol) and 2-bromoaniline (1.5 g, 8.7 mmol) in 20 mL of diisopropylethylamine was heated to 90° C. for 48 hours. The crude mixture was dissoloved in 200 mL of EtOAc and washed with water (1×150 mL), saturated aqueous NaHCO₃ (3×150 mL), and saturated aqueous NaCl (1×150 mL). The resulting organic solution was dried over MgSO₄, vacuum filtered, and concentrated to a brown oil. Purification by column chromatography on silica gel (EtOAc-hexanes, 3:7) afforded 0.59 g (23%) of an orange solid. ¹H NMR (400 MHz, CDCl₃) δ 8.08-8.04 (m, 1 H), 7.82-7.78 (m, 1 H), 7.67-7.60 (m, 2 H), 7.33 (dd, J=8.0, 1.6 Hz, 1 H), 7.12-7.08 (m, 1 H), 6.57-6.51 (m, 2 H), 5.65 (appar t, 1 H), 3.41-3.35 (m, 4 H). MS calculated for C₁₄H₁₅BrN₃O₄S+H: 400, observed: 400.

### Step 3: Preparation of 1-(2-Bromo-phenyl)-4-(2-nitro-benzenesulfonyl)-2-vinyl-piperazine

A solution of N-[2-(2-Bromo-phenylamino)-ethyl]-2-nitro-benzenesulfonamide (900 mg, 2.3 mmol), (Z)-2-butenylene dimethyl dicarbonate (700 mg, 3.4 mmol), and diphenyl-2-pyridylphosphine (100 mg, 0.4 mmol) in 20 mL of toluene was charged with tetrakis(triphenyl-phosphine)palladium (130 mg, 5 mole %) and heated 100° C. for 2 hours. Afterwards, the crude mixture was dissolved in 100 mL of EtOAc and washed 1 M HCl (3×100 mL) and saturated aqueous NaCl (1×100 mL). The resulting organic solution was dried over MgSO₄, vacuum filtered, and concentrated to a brown oil. Purification by column chromatography on silica gel (EtOAc-hexanes, 1:3) afforded 0.71 g (70%) of a yellow oil. ¹H NMR (400 MHz, CDCl₃) δ 7.99-7.96 (m, 1 H), 7.74-7.67 (m, 2 H), 7.63-7.61 (m, 1 H), 7.53 (dd, J=7.8, 1.4 Hz, 1 H), 7.21 (dd, J=7.2, 1.6 Hz, 1 H), 7.01 (dd, J=8.2, 1.4 Hz, 1 H), 6.95 (td, J=7.6, 1.6 Hz, 1 H), 5.54 (ddd, J=17.4, 10.6, 7.6 Hz, 1 H), 5.14 (d, J=17.6 Hz, 1 H), 5.08 (d, J=10.4 Hz, 1 H), 3.88 (td, J=8.0 Hz, 3.2 Hz, 1 H), 3.72-3.63 (m, 2 H), 3.35-3.28 (m, 2 H), 3.07 (br t, J=10.0 Hz, 1 H), 2.80-2.74 (m, 1 H). MS calculated for C₁₈H₁₉BrN₃O₄S+H: 452, observed: 452.

### Step 4: Preparation of 1-(2-Bromo-phenyl)-2-vinyl-piperazine trifluoroacetic acid salt

A solution of 1-(2-Bromo-phenyl)-4-(2-nitro-benzenesulfonyl)-2-vinyl-piperazine (510 mg, 1.1 mmol), benzenethiol (200 μL, 2.0 mmol), K₂CO₃ (400 mg, 2.9 mmol) in 5 mL of DMF was stirred for 8 hours. Afterwards, the crude mixture was dissolved in 150 mL of ether and washed with water (2×150 mL) and saturated aqueous NaCl (2×150 mL). The resulting organic solution was dried over MgSO₄, vacuum filtered, and concentrated to a brown oil. Purification by column reverse-phase HPLC (MeCN-water, 3:7) afforded 280 mg (81 %) of a white solid. ¹H NMR (400 MHz, CDCl₃) δ 9.95 (br s, 1 H), 9.70 (br s, 1 H), 7.56 (dd, J=7.8, 1.4 Hz, 1 H), 7.25 (td, J=7.8, 1.4 Hz, 1 H), 7.08 (dd, J=7.8, 1.4 Hz, 1 H), 7.00 (td, J=7.6, 1.5 Hz, 1 H), 5.49 (dt, J=17.6, 8.5 Hz, 1 H), 5.21 (d, J=17.2 Hz, 1 H), 5.12 (d, J=10.4 Hz, 1 H), 4.13-4.08 (m, 1 H), 3.41-3.30 (m, 4 H), 3.08-3.01 (m, 2 H). MS calculated for C₁₂H₁₅BrN₂: 266, observed: 266.

### Example 3.2

### Preparation of (R)-1-(4-Chloro-phenyl)-2-methyl-piperazine hydrochloride salt (Compound 2 HCl salt)

After a solution of 4-bromochlorobenzene (400 mg, 2.1 mmol), (R)-4-N-boc-2-methyl-piperazine (350 mg 1.8 mmol), 2-di-t-butyl phosphino-2′-(N,N-dimethylamino) biphenyl (20 mg, 3 mole %), and tris(dibenzylideneacetone) dipalladium (10 mg, 1 mole %) in 10 mL of dry THF was degassed with argon for 5 minutes, a 1 M solution of lithium bis(trimethylsilyl)amide (2.5 mL, 2.5 mmol) in THF was added as a single portion. The reaction mixture was then heated to 65° C. for 18 hours. Afterwards, the crude mixture was concentrated to a brown oil and purification by column chromatography on silica gel (EtOAc-hexanes, 1:4) afforded a yellow oil.

The resulting (R)-4-N-boc-1-(4-Chloro-phenyl)-2-methyl-phenylpiperazine was dissolved in a premixed solution of MeOH (20 mL, 250 mmol) and acetylchloride (1 mL, 14 mmol). After standing for 2 hours, the reaction mixture was concentrated to afforded 70 mg of a purple solid. ¹H NMR (400 MHz, CD₃OD) δ 7.35 (appar d, J=8.8 Hz, 2 H), 7.18 (appar d, J=8.8 Hz, 2 H), 3.97-3.90 (m, 1 H), 3.50-3.42 (m, 3 H), 3.40-3.33 (m, 2 H), 3.25 (dd, J=12.8, 6.8 Hz, 1 H), 1.08 (d, J=6.8 Hz, 3 H). MS calculated for C₁₁H₁₅ClN₂+H: 211, observed 211.

### Example 3.3

### Preparation of (R,S) 1-(4-Chloro-phenyl)-2-vinyl-piperazine (Compound 3)

By the same general procedure as in Example 3.1, 1-(4-Chloro-phenyl)-2-vinyl-piperazine was obtained from 4-chloroaniline as a colorless oil. ¹H NMR (400 MHz, CDCl₃), mixture of rotamers, δ 7.16 (d, J=9.2 Hz, 2 H), 6.80 (d, J=9.2 Hz, 2 H), 5.77 (ddd, J=17.4, 10.4, 6.2 Hz, 1 H), 5.15 (dt, J=11.1, 1.5 Hz, 1 H), 5.06 (dt, J=17.5, 1.3 Hz, 1 H), 4.07-4.03 (m, 1 H), 3.18-3.07 (m, 4 H), 3.07-3.03 (m, 1 H), 2.95 (ddd, J=12.0, 7.6, 5.2 Hz, 1 H), 1.70 (br s, 1 H). MS calculated for C₁₂H₁₅ClN₂+H: 223, observed 223.

### Example 3.4

### Preparation of (R,S) 1-(3-Fluoro-phenyl)-2-vinyl-piperazine (Compound 4)

By the same general procedure as in Example 3.1, 1-(3-Fluoro-phenyl)-2-vinyl-piperazine was obtained from 3-fluoroaniline as a colorless oil. ¹H NMR (400 MHz, CDCl₃), δ 7.14 (td, J=8.3, 7.5 Hz, 1 H), 6.61 (dd, J=8.8, 2.4 Hz, 1 H), 6.53 (dt, J=12.8, 2.4 Hz, 1 H), 6.49-6.44 (m, 1 H), 5.82 (ddd, J=17.4, 10.6, 6.0, 1 H), 5.19 (dt, J=10.7, 1.2 Hz, 1 H), 5.08 (dt, J=17.5, 1.5 Hz, 1 H), 4.16-4.15 (m, 1 H), 3.26 (dt, J=11.9, 3.7 Hz, 1 H), 3.18-3.07 (m, 4 H), 2.94 (ddd, J=12.4, 10.4, 3.6 Hz, 1 H), 1.77 (br s, 1 H). MS calculated for C₁₂H₁₅FN₂+H: 207, observed 207.

### Example 3.5

### Preparation of (R,S) 1-(3-Chloro-4-fluoro-phenyl)-2-vinyl-piperazine (Compound 5)

By the same general procedure as in Example 3.1, 1-(3-Chloro-4-fluoro-phenyl)-2-vinyl-piperazine was obtained from 3-Chloro-4-fluoroaniline as a colorless oil. ¹H NMR (400 MHz, CDCl₃), mixture of rotamers, δ 6.99 (q, J=8.5 Hz, 1 H), 6.91-6.88 (m, 1 H), 6.77-6.72 (m, 1 H), 5.75 (ddd, J=17.4, 10.6, 6.4 Hz, 1 H), 5.14 (appar dt, J=10.4 Hz, 1 H), 5.06 (dt, J=17.2, 1.0 Hz, 1 H), 3.94-3.91 (m, 1 H), 3.17-2.92 (m, 6 H), 1.72 (br, s, 1 H). MS calculated for C₁₂H₁₄ClFN₂+H: 241, observed 241.

### Example 3.6

### Preparation of (R,S) 1-(3-Chloro-phenyl)-2-vinyl-piperazine (Compound 6)

By the same general procedure as in Example 3.1, 1-(3-Chloro-phenyl)-2-vinyl-piperazine was obtained from 3-chloroanilne as a colorless oil. ¹H NMR (400 MHz, CDCl₃), mixture of rotamers, δ 7.11 (t, J=8.0 Hz, 1 H), 6.82 (t, J=2.0 Hz, 1 H), 6.75-6.71 (m, 2 H), 5.80 (ddd, J=17.4, 10.8, 5.8 Hz, 1 H), 5.18 (dt, J=10.4, 1.5 Hz, 1 H), 5.07 (dt, J=17.5, 1.5 Hz, 1 H), 4.14-4.13 (m, 1 H), 3.24 (dt, J=12.4, 3.5 Hz, 1 H), 3.17-3.04 (m, 4 H), 2.96,2.90 (m, 1 H), 1.74 (br s, 1 H). MS calculated for C₁₂H₁₅ClN₂+H: 223, observed 223.

### Example 3.7

### Preparation of (R,S) 1-(3-Bromo-phenyl)-2-vinyl-piperazine trifluoroacetic acid salt (Compound 7 TFA salt)

By the same general procedure as in Example 3.1, 1-(3-bromo-phenyl)-2-vinyl-piperazine trifluoroacetic acid was obtained from 3-bromoaniline as a white solid. ¹H NMR (400 MHz, CDCl₃), mixture of rotamers, δ 9.87 (br s, 1 H), 9.70 (br s, 1 H), 7.14-7.12 (m, 2 H), 7.10 (s, 1 H), 6.91-6.86 (m, 1 H), 5.70 (ddd, J=17.0, 10.6, 7.2 Hz, 1 H), 5.25 (d, J=10.4 Hz, 1 H), 5.22 (d, J=17.6 Hz, 1 H), 4.18-4.14 (m, 1 H), 3.42-3.29 (m, 5 H), 3.19 (dd, J=12.4, 6.4 Hz, 1 H). MS calculated for C₁₂H₁₅BrN₂+H: 267, observed 267.

### Example 3.8

### Preparation of (R,S) 1-(3,5-Dichloro-phenyl)-2-vinyl-piperazine trifluoroacetic acid salt (Compound 8 TFA salt)

By the same general procedure as in Example 3.1, 1-(3,5-dichloro-phenyl)-2-vinyl-piperazine trifluoroacetic acid was obtained from 3,5-dichloroaniline as a white solid. MS calculated for C₁₂H₁₄Cl₂N₂+H: 257, observed 257.

### Example 3.9

### Preparation of (R,S) 1-(2-Bromo-4-isopropyl-phenyl)-2-vinyl-piperazine trifluoracetic acid salt (Compound 9 TFA salt)

By the same general procedure as in Example 3.1, 1-(2-bromo-4-isopropyl-phenyl)-2-vinyl-piperazine trifluoracetic acid was obtained from 2-bromo-4-isopropylaniline as a white solid. ¹H NMR (400 MHz, CDCl₃) δ 7.41 (d, J=2.0 Hz, 1 H), 7.09 (dd, J=8.4, 2.0 Hz, 1 H), 6.99 (d, J=8.0 Hz, 1 H), 5.52-5.43 (m, 1 H), 5.21 (d, J=17.2 Hz, 1 H), 5.11 (d, J=10.8 Hz, 1 H), 4.09-4.04 (m, 1 H), 3.39-3.26 (m, 4 H), 3.07-2.98 (m, 2 H), 2.83 (septet, J=6.9 Hz, 1 H), 1.21 (d, J=6.8 Hz, 6H).

### Example 3.10

### Preparation of (R,S) 1-(2-Bromo-4-trifluoromethoxy-phenyl)-2-vinyl-piperazine trifluoroacetic acid salt (Compound 10 TFA salt)

By the same general procedure as in Example 3.1, 1-(2-Bromo-4-trifluoromethoxy-phenyl)-2-vinyl-piperazine trifluoroacetic acid was obtained from 2-bromo-4-trifluoro-methoxyaniline as a white solid. ¹H NMR (400 MHz, CDCl₃) δ 9.96 (br s, 1 H), 9.63 (br s, 1 H), 7.46 (d, J=1.6 Hz, 1 H), 7.14 (dd, J=8.8, 2.0 Hz, 1 H), 7.10 (t, J=8.8 Hz, 1 H), 5.46 (dt, J=17.2, 8.5 Hz, 1 H), 5.23 (d, J=16.8 Hz, 1 H), 5.16 (d, J=10.0 Hz, 1 H), 4.07 (t, J=8.0 Hz, 1 H), 3.42-3.28 (m, 4 H), 3.07-3.02(m,2H).

### Example 3.11

### Preparation of (R,S) 1-(2-Bromo-4-trifluoromethyl-phenyl)-2-vinyl-piperazine trifluoro-acetic acid salt (Compound 11 TFA salt)

By the same general procedure as in Example 3.1, 1-(2-Bromo-4-trifluoromethyl-phenyl)-2-vinyl-piperazine trifluoroacetic acid was obtained from 2-bromo-4-trifluoro-methylaniline as a white solid. MS calculated for C₁₃H₁₄BrF₃N₂+H: 335, observed 335.

### Example 3.12

### Preparation of (R,S) 3-(2-Methyl-piperazin-1-yl)-benzonitrile trifluoroacetic acid salt (Compound 12 TFA salt)

By the same general procedure as in Example 3.1, 3-(2-Methyl-piperazin-1-yl)-benzonitrile trifluoroacetic acid salt was obtained from 3-aminobenzonitrile as a white solid. ¹H NMR (400 MHz, CDCl₃), δ 7.37 (t, J=7.8 Hz, 1 H) 7.27 (dd, J=7.6, 0.8 Hz, 1 H), 7.19-7.17 (m, 2 H), 5.71 (ddd, J=17.0, 10.4, 7.0 Hz, 1 H), 5.29 (d, J=10.8 Hz, 1 H), 5.24 (d, J=17.2 Hz, 1 H), 4.25-4.21 (m, 1 H, 3.46-3.39 (m, 4 H), 3.36-3.33 (m, 1 H), 3.25 (dd, J=12.8, 6.4 Hz, 1 H).

### Example 3.13

### Preparation of (R,S) 1-(3,5-difluoro-phenyl)-2-vinyl-piperazine (Compound 13)

By the same general procedure as in Example 3.1, 1-(3,5-difluoro-phenyl)-2-vinyl-piperazine was obtained from 3,5-difluoroaniline as a white solid. ¹H NMR (400 MHz, CDCl₃) δ 6.44-6.38 (m, 3 H), 5.79 (ddd, J=17.2, 10.6, 6.6 Hz, 1 H), 5.33 (d, J=10.4 Hz, 1 H), 5.25 (d, J=17.2 Hz, 1 H), 4.28-4.24 (m, 1 H), 3.46-3.37 (m, 4 H), 3.32-3.23 (m, 2 H), 2.25 (br s, 1 H).

### Example 3.14

### Preparation of (R,S) 1-o-Tolyl-2-vinyl-piperazine trifluoroacetic acid salt (Compound 14 TFA salt)

By the same general procedure as in Example 3.1, 1-o-Tolyl-2-vinyl-piperazine trifluoroacetic acid salt was obtained from o-toluidine as a white solid. ¹H NMR (400 MHz, CD₃OD), mixture of rotamers, δ 7.19 (d, J=7.6 Hz, 1 H), 7.13-7.10 (m, 2 H), 7.04 (dd, J=7.6, 2.8 Hz, 1 H), 5.45 (ddd, J=17.2, 10.4, 7.6 Hz, 1 H), 5.20 (d, J=17.2 Hz, 1 H), 5.07 (d, J=10.4 Hz, 1 H), 3.93 (ddd, J=10.4, 7.6, 2.8 Hz, 1 H), 3.42-3.31 (m, 3 H), 3.15-3.06 (m, 2 H), 3.00-2.95 (m, 1 H), 2.34 (s, 3 H).

### Example 3.15

### Preparation of (R,S) 1-(2,3-Difluoro-phenyl)-2-vinyl-piperazine hydrochloride salt (Compound 15 HCl salt)

By the same general procedure as in Example 3.1, 1-(2,3-difluoro-phenyl)-2-vinyl-piperazine was obtained from 2,3-difluoroaniline as a white solid. ¹H NMR (400 MHz, CDCl₃) δ 7.10-6.98 (m, 3 H), 5.70-5.64 (m, 1 H), 5.30 (d, J=17.2 Hz, 1 H), 5.21 (dd, J=10.2, 0.6 Hz, 1 H), 4.09 (td, J=8.1, 3.2 Hz, 1 H), 3.43-3.35 (m, 4 H), 3.28-3.16 (m, 2 H). MS calculated for C₁₂H₁₄F₂N₂+H: 225, observed 225.

### Example 3.16

### Preparation of (R,S) 1-(2,3-Difluoro-phenyl)-2-ethyl-piperazine trifluoroacetic acid salt (Compound 16 TFA salt)

By the same general procedure as in Example 3.1, 1-(2,3-difluoro-phenyl)-2-vinyl-piperazine was obtained from 2,3-difluoroaniline as a white solid. Further reduction of 1-(2,3-difluoro-phenyl)-2-vinyl-piperazine trifluoroacetic acid salt with palladium on activated carbon in MeOH while over a H₂ balloon afforded 1-(2,3-difluoro-phenyl)-2-ethyl-piperazine trifluoroacetic acid salt. ¹H NMR (400 MHz, CD₃OD) δ 7.14-7.07 (m, 1 H), 7.05-6.96 (m, 2 H), 3.52-3.43 (m, 2 H), 3.38-3.21 (m, 4 H), 3.15 (dd, J=12.4, 7.2 Hz, 1 H), 1.53 (quintet, J=7.2 Hz, 2 H), 0.84 (t, J=7.4 Hz, 3 H). MS calculated for C₁₂H₁₆F₂N₂+H: 227, observed 227.

### Example 3.17

### Preparation of (R,S) 1-(3-Fluoro-phenyl)-2-ethyl-piperazine trifluoroacetic acid salt (Compound 17 TFA salt)

By the same general procedure as in Example 3.1, 1-(3-fluoro-phenyl)-2-vinyl-piperazine was obtained from 3-fluoroaniline as a white solid. Further reduction of 1-(3-fluoro-phenyl)2-vinyl-piperazine trifluoroacetic acid salt with palladium on activated carbon in MeOH while over a H₂ balloon afforded 1-(3-fluoro-phenyl)-2-ethyl-piperazine trifluoroacetic acid salt. ¹H NMR (400 MHz, CDCl₃) δ 10.43 (br s, 1 H), 9.67 (br s, 1 H), 9.32 (br s, 1 H), 7.25 (t, J=7.4 Hz, 1 H), 7.23 (t, J=7.4 Hz, 1 H), 6.72-6.62 (m, 2 H), 3.71-3.66 (m, 1 H), 3.45-3.33 (m, 5 H), 3.24-3.20 (m, 1 H), 1.75-1.57 (m, 2 H), 0.88 (t, J=7.2 Hz, 3 H). MS calculated for C₁₂H₁₇FN₂+H: 209, observed 209.

### Example 3.18

### Preparation of (R,S) 1-(4-Fluoro-phenyl)-2-ethyl-piperazine trifluoroacetic acid salt (Compound 18 TFA salt)

By the same general procedure as in Example 3.1, 1-(3-fluoro-phenyl)-2-vinyl-piperazine was obtained from 4-fluoroaniline as a white solid. Further reduction of 1-(4-fluoro-phenyl)-2-vinyl-piperazine trifluoroacetic acid salt with palladium on activated carbon in MeOH while over a H₂ balloon afforded 1-(4-fluoro-phenyl)-2-ethyl-piperazine trifluoroacetic acid salt. MS calculated for C₁₂H₁₇FN₂+H: 209, observed 209.

### Example 3.19

### Preparation of (R)-1-(3-Chloro-4-fluoro-phenyl)-2-methyl-piperazine hydrochloride salt (Compound 19 HCl salt)

By the same general procedure as in Example 3.2, (R)-1-(3-Chloro-4-fluoro-phenyl)-2-methyl-piperazine was obtained from 4-bromo-2chloro-1-fluorobenzene as a light brown solid. ¹H NMR (400 MHz, CD₃OD) δ 7.20-7.15 (m, 2 H), 7.02 (ddd, J=9.2, 3.8, 2.6 Hz, 1 H), 3.76-3.68 (m, 1 H), 3.40 (dd, J=12.6, 3.4 Hz, 1 H), 3.34-3.29 (m, 2 H), 3.27-3.22 (m, 2 H), 3.11 (dd, J=12.6, 6.2 Hz, 1 H), 1.03 (d, J=6.8 Hz, 3 M). MS calculated for C₁₁H₁₄ClFN₂+H: 229, observed 229.

### Example 3.20

### Preparation of (S)-1-(3-Chloro-4-fluoro-phenyl)-2-methyl-piperazine hydrochloride salt (Compound 20 HCl salt)

By the same general procedure as in Example 3.2, (S)-1-(3-Chloro-4-fluoro-phenyl)-2-methyl-piperazine was obtained from 4-bromo-2chloro-1-fluorobenzene and (S)-4-N-boc-2-methyl-piperazine as a light brown solid. ¹H NMR (400 MHz, CDCl₃) δ 9.49 (br s, 1 H), 7.12-7.07 (m, 2 H), 6.94 (ddd, J=9.0, 3.8, 2.8 Hz, 1 H), 3.60-3.53 (m, 1 H), 3.39 (dd, J=12.4, 3.4 Hz, 1 H), 3.33-3.32 (m, 2 H), 3.28-3.18 (m, 2 H), 3.03 (dd, J=12.4, 8.0 Hz, 1 H), 1.02 (dd, J=6.4 Hz, 3 H). MS calculated for C₁₁H₁₄ClFN₂+H: 229, observed 229.

### Example 3.21

### Preparation of (R)-1-(3,4-Difluoro-phenyl)-2-methyl-piperazine trifluoroacetic acid salt (Compound 21 TFA salt)

By the same general procedure as in Example 3.2, (R)-1-(3,4-difluoro-phenyl)-2-methyl-piperazine was obtained from 1-bromo-2,3-difluorobenzene as a white solid. ¹H NMR (400 MHz, CD₃OD) δ 7.15-7.05 (m, 2 H), 7.04-6.99 (m, 1 H), 3.62-3.54 (m, 1 H), 3.44-3.40 (m, 1 H), 3.38-3.29 (m, 3 H), 3.28-3.18 (m, 1 H), 3.01 (dd, J=12.6, 8.6 Hz, 1 H), 1.02 (d, J=6.4 Hz, 3 H). MS calculated for C₁₁H₁₄ClFN₂+H: 213, observed 213.

### Example 3.22

### Preparation of (S)-1-(3,4-Difluoro-phenyl)-2-methyl-piperazine hydrochloride salt (Compound 22 HCl salt)

By the same general procedure as in Example 3.2, (S)-1-(3,4-difluoro-phenyl)-2-methyl-piperazine was obtained from 1-bromo-2,3-difluorobenzene and (S)-4-N-boc-2-methyl-piperazine as a white solid. ¹H NMR (400 MHz, CD₃OD) δ 7.27-7.12 (m, 2 H), 6.99 (br m, 1 H), 3.89-3.83 (m, 1 H), 3.47-3.33 (m, 5 H), 3.21 (dd, J=12.8, 6.4 Hz, 1 H), 1.06 (dd, J=6.4, 1.6 Hz, 3 H). MS calculated for C₁₁H₁₄F₂N₂+H: 213, observed 213.

### Example 3.23

### Preparation of (R)-1-(3-Chloro-2-fluoro-phenyl)-2-methyl-piperazine hydrochloride salt (Compound 23 HCl salt)

By the same general procedure as in Example 3.2, (R)-1-(3-Chloro-2-fluoro-phenyl)-2-methyl-piperazine was obtained from 3-chloro-2-fluoroiodobenzene as a white solid. ¹H NMR (400 MHz, CD₃OD) δ 7.28-7.24 (m, 1 H), 7.20-7.16 (m, 1 H), 7.13-7.09 (m, 1 H), 3.61-3.53 (m, 1 H), 3.40 (dd, J=12.6,3.0 Hz, 1 H), 3.39-3.26 (m, 3 H), 3:23-3.18 (m, 1 H), 2.98 (dd, J=12.4, 8.8 Hz, 1 H), 0.97 (d, J=6.4 Hz, 3 H). MS calculated for C₁₁H₁₄ClFN₂+H: 229, observed 229.

### Example 3.24

### Preparation of (S)-1-(3-Chloro-2-fluoro-phenyl)-2-methyl-piperazine hydrochloride salt (Compound 24 HCl salt)

By the same general procedure as in Example 3.2, (S)-1-(3-Chloro-2-fluoro-phenyl)-2-methyl-piperazine was obtained from 3-chloro-2-fluoroiodobenzene and (S)-4-N-boc-2-methyl-piperazine as a white solid. ¹H NMR (400 MHz, CD₃OD) δ 7.22-7.28 (m, 1 H), 7.23-7.18 (m, 1 H), 7.19-7.13 (m, 1 H), 3.63-3.56 (m, 1 H), 3.44 (dd, J=12.4, 2.8 Hz, 1 H), 3.40-3.30 (m, 3 H), 3.26-3.22 (m, 1 H), 3.01 (dd, J=12.2, 9.0 Hz, 1 H), 1.01 (d, J=6.4 Hz, 3 H). MS calculated for C₁₁H₁₄ClFN₂+H: 229, observed 229.

### Example 3.25

### Preparation of (R)-1-(3,5-Difluoro-phenyl)-2-methyl-piperazine trifluoroacetic acid salt (Compound 25 TFA salt)

By the same general procedure as in Example 3.2, (R)-1-(3,5-difluoro-phenyl)-2-methyl-piperazine was obtained from 1-bromo-2,4-difluorobenzene as a yellow oil. ¹H NMR (400 MHz, CD₃OD) δ 6.58 (dd, J=10.8, 2.0 Hz, 2 H), 6.41 (m, J=8.9, 2.3 Hz, 1 H), 4.23-4.16 (m, 1 H), 3.58-3.50 (m, 1 H), 3.43-3.17 (m, 5 H), 1.17 (d, J=6.4 Hz, 3 H). MS calculated for C₁₁H₁₄F₂N₂+H: 213, observed 213.

### Example 3.26

### Preparation of (S)-1-(3,5-Difluoro-phenyl)-2-methyl-piperazine trifluoroacetic acid salt (Compound 26 TFA salt)

By the same general procedure as in Example 3.2, (S)-1-(3,5-difluoro-phenyl)-2-methyl-piperazine was obtained from 1-bromo-2,4-difluorobenzene and (S)-4-N-boc-2-methyl-piperazine as a brown oil. ¹H NMR (400 MHz, CD₃OD) δ 6.58 (dd, J=10.8, 2.0 Hz, 2 H), 6.41 (tt, J=8.9, 2.3 Hz, 1 H), 4.24-4.17 (m, 1 H), 3.58-3.50 (m, 1 H), 3.42-3.17 (m, 5 H), 1.17 (d, J=6.4 Hz, 3 H). MS calculated for C₁₁H₁₄F₂N₂+H: 213, observed 213.

### Example 3.27

### Preparation of (S)-1-(4-Chloro-phenyl)-2-methyl-piperazine hydrochloride salt (Compound 27 HCl salt)

By the same general procedure as in Example 3.2, (S)-1-(4-chloro-phenyl)-2-methyl-piperazine was obtained from 1-bromo-4-chlorobenzene and (S)-4-N-boc-2-methyl-piperazine as a purple solid. ¹H NMR (400 MHz, CD₃OD) δ 7.33 (appar d, J=9.2 Hz, 2 H), 7.13 (appar d, J=8.8 Hz, 2 H), 3.95-3.88 (m, 1 H), 3.48-3.38 (m, 3 H), 3.36-3.30 (m, 2 H), 3.22 (dd, J=12.8, 6.4 Hz, 1 H), 1.07 (d, J=6.8 Hz, 3 H). MS calculated for C₁₁H₁₅ClN₂+H: 211, observed 211.

### Example 3.28

### Preparation of (R)-1-(4-Fluoro-phenyl)-2-methyl-piperazine hydrochloride salt (Compound 28 HCl salt)

By the same general procedure as in Example 3.2, (R)-1-(4-fluoro-phenyl)-2-methyl-piperazine was obtained from 1-bromo-4-fluorobenzene as a brown solid. ¹H NMR (400 MHz, CD₃OD) δ 7.47-7.44 (m, 2 H), 7.21 (appar t, J=8.8 Hz, 2 H), 3.92-3.86 (m, 1 H), 3.61-3.50 (m, 5 H), 3.34 (dd, J=8.8, 4.4 Hz, 1 H), 1.08 (d, J=6.4 Hz, 3 H). MS calculated for C₁₁H₁₅FN₂+H: 195, observed 195.

### Example 3.29

### Preparation of (S)-1-(4-Fluoro-phenyl)-2-methyl-piperazine hydrochloride salt (Compound 29 HCl salt)

By the same general procedure as in Example 3.2, (S)-1-(4-fluoro-phenyl)-2-methyl-piperazine was obtained from 1-bromo-4-fluorobenzene and (S)-4-N-boc-2-methyl-piperazine as a brown solid. ¹H NMR (400 MHz, CD₃OD) δ 7.47-7.44 (m, 2 H), 7.21 (appar t, J=8.8 Hz, 2 H), 3.92-3.86 (m, 1 H), 3.61-3.50 (m, 5 H), 3.34 (dd, J=8.8, 4.4 Hz, 1 H), 1.08 (d, J=6.4 Hz, 3 H). MS calculated for C₁₁H₁₅FN₂+H: 195, observed 195.

### Example 3.30

### Preparation of (R)-1-(3,4-Dichloro-phenyl)-2-methyl-piperazine hydrochloride salt (Compound 30 HCl salt)

By the same general procedure as in Example 3.2, (R)-1-(3,4-dichloro-phenyl)-2-methyl-piperazine was obtained from 1-bromo-3,4-dichlorobenzene as a yellow solid. ¹H NMR (400 MHz, CD₃OD) δ 7.38 (d, J=8.8 Hz, 1 H), 7.22 (d, J=2.4 Hz, 1 H), 7.00 (dd, J=8.8, 2.8 Hz, 1 H), 4.06-3.98 (m, 1 H), 3.47-3.36 (m, 3 H), 3.28-3.21 (m, 2 H), 1.07 (d, J=6.4 Hz, 3 H). MS calculated for C₁₁H₁₄Cl₂N₂+H: 245, observed 245.

### Example 3.31

### Preparation of (S)-1-(3,4-Dichloro-phenyl)-2-methyl-piperazine hydrochloride salt (Compound 31 HCl salt)

By the same general procedure as in Example 3.2, (S)-1-(3,4-dichloro-phenyl)-2-methyl-piperazine was obtained from 1-bromo-3,4-dichlorobenzene and (S)-4-N-boc-2-methyl-piperazine as a purple solid. ¹H NMR (400 MHz, CD₃OD) δ 7.38 (d, J=8.8 Hz, 1 H), 7.22 (d, J=2.4 Hz, 1 H), 7.00 (dd, J=8.8, 2.8 Hz, 1 H), 4.06-3.98 (m, 1 H), 3.47-3.36 (m, 3 H), 3.28-3.21 (m, 2 H), 1.07 (d, J=6.4 Hz, 3 H). MS calculated for C₁₁H₁₄Cl₂N₂+H: 245, observed 245.

### Example 3.32

### Preparation of (R)-1-(3-Chloro-4-methyl-phenyl)-2-methyl-piperazine hydrochloride salt (Compound 32 HCl salt)

By the same general procedure as in Example 3.2, (R)-1-(3-Chloro-4-methyl-phenyl)-2-methyl-piperazine was obtained from 4-bromo-2-chlorotoluene as a purple solid. ¹H NMR (400 MHz, CD₃OD) δ 7.30 (d, J=8.0 Hz, 1 H), 7.26 (d, J=2.4 Hz, 1 H), 7.09 (dd, J=8.2, 2.2 Hz, 1 H), 3.96-3.88 (m, 1 H), 3.52-3.37 (m, 5 H), 3.27 (dd, J=12.8, 7.2 Hz, 1 H), 2.33 (s, 3 H), 1.09 (d, J=6.8 Hz, 3 H). MS calculated for C₁₂H₁₇ClN₂+H: 225, observed 225.

### Example 3.33

### Preparation of (S)-1-(3-Chloro-4-methyl-phenyl)-2-methyl-piperazine hydrochloride salt (Compound 33 HCl salt)

By the same general procedure as in Example 3.2, (S)-1-(3-Chloro-4-methyl-phenyl)-2-methyl-piperazine was obtained from 4-bromo-2-chlorotoluene and (S)-4-N-boc-2-methyl-piperazine as a purple solid. ¹H NMR (400 MHz, CD₃OD) δ 7.50 (d, J=2.0 Hz, 1 H), 7.39 (d, J=8.4 Hz, 1 H), 7.30 (dd, J=8.0, 2.0 Hz, 1 H), 4.11-4.03 (m, 1 H), 3.71-3.49 (m, 5 H), 3.43 (dd, J=13.2, 8.8 Hz, 1 H), 2.37 (s, 3 H), 1.14 (d, J=6.8 Hz, 3 H). MS calculated for C₁₂H₁₇ClN₂+H: 225, observed 225.

### Example 3.34

### Preparation of (R)-1-(3,4-Difluoro-phenyl)-2-methyl-piperazine trifluoroacetic acid salt (Compound 34 TFA salt)

By the same general procedure as in Example 3.2, (R)-1-(3,4-difluoro-phenyl)-2-methyl-piperazine was obtained from l-bromo-3,4-difluorobenzene as a tan solid. ¹H NMR (400 MHz, CD₃OD) δ 7.17 (q, J=9.6 Hz, 1 H), 7.00 (ddd, J=12.8, 7.2, 2.8 Hz, 1 H), 6.86-6.82 (m, 1 H), 3.80-3.73 (m, 1 H), 3.40 (dd, J=12.4, 3.6 Hz, 1 H), 3.36-3.32 (m, 2 H), 3.27-3.19 (m, 2 H), 3.14 (dd, J=12.4, 6.0 Hz, 1 H), 1.04 (d, J=6.4 Hz, 3 H). MS calculated for C₁₁H₁₄F₂N₂+H: 213, observed 213.

### Example 3.35

### Preparation of (S)-1-(3,4-Difluoro-phenyl)2-methyl-piperazine hydrochloride salt (Compound 35 HCl salt)

By the same general procedure as in Example 3.2, (S)-1-(3,4-difluoro-phenyl)-2-methyl-piperazine was obtained from 1-bromo-3,4-difluorobenzene and (S)-4-N-boc-2-methyl-piperazine as a tan solid. ¹H NMR (400 MHz, CD₃OD) δ 7.25 (q, J=9.5 Hz, 1 H), 7.22-7.18 (m, 1 H), 7.04-7.02 (m, 1 H), 3.92-3.85 (m, 1 H), 3.49-3.42 (m, 3 H), 3.39-3.33 (m, 2 H), 3.25 (dd, J=12.8, 7.2 Hz, 1 H), 1.06 (d, J=6.8 Hz, 3 H). MS calculated for C₁₁H₁₄F₂N₂+H: 213, observed 213.

### Example 3.36

### Preparation of (R)-1-(3,5-Dichloro-phenyl)-2-methyl-piperazine hydrochloride salt (Compound 36 HCl salt)

By the same general procedure as in Example 3.2, (R)-1-(3,5-dichloro-phenyl)-2-methyl-piperazine was obtained from 1-bromo-3,5-dichlorobenzene as a light brown solid. ¹H NMR (400 MHz, CD₃OD) δ 6.95 (d, J=1.6 Hz, 2 H), 6.88 (d, J=1.2 Hz, 1 H), 4.09-4.07 (m, 1 H), 3.47-3.44 (m, 1 H), 3.37-3.29 (m, 2 H), 3.25-3.14 (m, 3 H), 1.05 (d, J=6.8 Hz, 3 H). MS calculated for C₁₁H₁₄Cl₂N₂+H: 245, observed 245.

### Example 3.37

### Preparation of (S)-1-(3,5-Dichloro-phenyl)-2-methyl-piperazine hydrochloride salt (Compound 37 HCl salt)

By the same general procedure as in Example 3.2, (S)-4-(3,5-dichloro-phenyl)-2-methyl-piperazine was obtained from 1-bromo-3,5-dichlorobenzene and (S)-4-N-boc-2-methyl-piperazine as a light brown solid. ¹H NMR (400 MHz, CD₃SOCD₃) δ 9.59 (br s, 1 H), 9.09 (br, s, 1 H), 6.97 (s, 2 H), 6.93 (s, 1 H), 3.62 (d, J=13.6 Hz, 1 H), 3.27-3.13 (m, 5 H), 3.03-2.97 (m, 1 H), 1.14 (d, J=6.8 Hz, 3 H). MS calculated for C₁₁H₁₄Cl₂N₂+H: 245, observed 245.

### Example 3.38

### Preparation of (R)-1-(2,5-Difluoro-phenyl)-2-methyl-piperazine trifluoroacetic acid salt (Compound 38 TFA salt)

By the same general procedure as in Example 3.2, (R)-1-(2,5-difluoro-phenyl)-2-methyl-piperazine was obtained from 1-bromo-2,5-difluorobenzene as a light brown solid. ¹H NMR (400 MHz, CD₃OD) δ 7.13 (ddd, J=11.2, 9.0, 5.0 Hz, 1 H), 7.00 (ddd, J=9.6, 6.4, 3.2 Hz, 1 H), 6.94-6.89 (m, 1 H), 3.66-3.59 (m, 1 H), 3.42 (dd, J=12.4, 2.8 Hz, 1 H), 3.37-3.29 (m, 3 H), 3.24-3.17 (m, 1 H), 3.02 (dd, J=12.4, 8.4 Hz, 1 H), 1.03 (d, J=6.4 Hz, 3 H). MS calculated for C₁₁H₁₄F₂N₂+H: 213, observed 213.

### Example 3.39

### Preparation of (S)-1-(2,5-Difluoro-phenyl)-2-methyl-piperazine hydrochloride salt (Compound 39 HCl salt)

By the same general procedure as in Example 3.2, (S)-4-(2,5-difluoro-phenyl)-2-methyl-piperazine was obtained from 1-bromo-2,5-difluorobenzene and (S)-4-N-boc-2-methyl-piperazine as a light brown solid. ¹H NMR (400 MHz, CD₃OD) δ 7.13 (ddd, J=11.2, 9.0, 5.0 Hz, 1 H), 7.00 (ddd, J=9.6, 6.4, 3.2 Hz, 1 H), 6.94-6.89 (m, 1 H), 3.66-3.59 (m, 1 H), 3.42 (dd, J=12.4, 2.8 Hz, 1 H), 3.37-3.29 (m, 3 H), 3.24-3.17 (m, 1 H), 3.02 (dd, J=12.4, 8.4 Hz, 1 H), 1.03 (d, J=6.4 Hz, 3 H). MS calculated for C₁₁H₁₄F₂N₂+H: 213, observed 213.

### Example 3.40

### Preparation of (R)-1-(4-Chloro-3-fluoro-phenyl)-2-methyl-piperazine hydrochloride salt (Compound 40 HCl salt)

By the same general procedure as in Example 3.2, (R)-1-(4-Chloro-3-fluoro-phenyl)-2-methyl-piperazine was obtained from 1-bromo-4-chloro-3-fluorobenzene as a white solid. ¹H NMR (400 MHz, CD₃OD) δ 7.36 (t, J=8.8 Hz, 1 H), 6.98 (dd, J=12.0, 2.8 Hz, 1 H), 6.87 (dd, J=8.8 Hz, 1 H), 4.13-4.06 (m, 1 H), 3.52-3.40 (m, 3 H), 3.33-3.24 (m, 3 H), 1.14 (d, J=6.8 Hz, 3 H). MS calculated for C₁₁H₁₄ClFN₂+H: 229, observed 229.

### Example 3.41

### Preparation of (S)-1-(4-Chloro-3-fluoro-phenyl)-2-methyl-piperazine hydrochloride salt (Compound 41 HCl salt)

By the same general procedure as in Example 3.2, (S)-1-(4-Chloro-3-fluoro-phenyl)-2-methyl-piperazine was obtained from 1-bromo-4-chloro-3-fluorobenzene and (S)-4-N-boc-2-methyl-piperazine as a white solid. ¹H NMR (400 MHz, CD₃OD) δ 7.35 (t, J=8.6 Hz, 1 H), 6.95 (dd, J=12.0, 2.8 Hz, 1 H), 6.87-6.84 (m, 1 H), 4.12-4.05 (m, 1 H), 3.50-3.39 (m, 3 H), 3.33-3.25 (m, 3 H), 1.14 (d, J=6.8 Hz, 3 H). MS calculated for C₁₁H₁₄ClFN₂+H: 229, observed 229.

### Example 3.42

### Preparation of (R)-1-(3-Chloro-2-methyl-phenyl)-2-methyl-piperazine hydrochloride salt (Compound 42 HCl salt)

By the same general procedure as in Example 3.2, (R)-1-(3-Chloro-2-methyl-phenyl)-2-methyl-piperazine was obtained from 2-bromo-6-chlorotoluene as a brown solid. ¹H NMR (400 MHz, CD₃OD) δ 7.27-7.18 (m, 3 H), 3.45-3.30 (m, 4 H), 3.08-2.94 (m, 3 H), 2.41 (s, 3 H), 0.89 (dd, J=6.0 Hz, 3 H). MS calculated for C₁₂H₁₇ClN₂+H: 225, observed 225.

### Example 3.43

### Preparation of (S)-1-(3-Chloro-2-methyl-phenyl)-2-methyl-piperazine hydrochloride salt (Compound 43 HCl salt)

By the same general procedure as in Example 3.2, (S)-1-(3-Chloro-2-methyl-phenyl)-2-methyl-piperazine was obtained from 2-bromo-6-chlorotoluene and (S)-4-N-boc-2-methyl-piperazine as a brown solid. ¹H NMR (400 MHz, CD₃OD) δ 7.27-7.18 (m, 3 H), 3.45-3.30 (m, 4 H), 3.07-2.92 (m, 3 H), 2.41 (s, 3 H), 0.89 (d, J=6.0 Hz, 3 H). MS calculated for C₁₂H₁₇ClN₂+H: 225, observed 225.

### Example 3.44

### Preparation of (R)-1-(5-Chloro-2-fluoro-phenyl)-2-methyl-piperazine hydrochloride salt (Compound 44 HCl salt)

By the same general procedure as in Example 3.2, (R)-1-(5-Chloro-2-fluoro-phenyl)-2-methyl-piperazine was obtained from 2-bromo-4-chloro-1-fluorobenzene as a white solid. ¹H NMR (400 MHz, CD₃OD) δ 7.26 (dd, J=6.8, 2.4 Hz, 1 H), 7.19 (ddd, J=8.8, 4.2, 2.6 Hz, 1 H), 7.14 (dd, J=11.2, 8.8 Hz, 1 H), 3.66-3.59 (m, 1 H), 3.43 (dd, J=12.6, 3.0 Hz, 1 H), 3.40-3.30 (m, 3 H), 3.25-3.18 (m, 1 H), 3.02 (dd, J=12.4, 8.4 Hz, 1 H), 1.03 (d, J=6.4 Hz, 3 H). MS calculated for C₁₁H₁₄ClFN₂+H: 229, observed 229.

### Example 3.45

### Preparation of (S)-1-(5-Chloro-2-fluoro-phenyl)-2-methyl-piperazine hydrochloride salt (Compound 45 HCl salt)

By the same general procedure as in Example 3.2, (S)-1-(5-Chloro-2-fluoro-phenyl)-2-methyl-piperazine was obtained from 2-bromo-4-chloro-1-fluorobenzene and (S)-4-N-boc-2-methyl-piperazine as a white solid. ¹H NMR (400 MHz, CD₃OD) δ 7.25 (dd, J=7.2, 2.4 Hz, 1 H), 7.22-7.18 (m, 1 H), 7.14 (dd, J=11.0, 9.0 Hz, 1 H), 3.63-3.60 (m, 1 H), 3.42 (dd, J=12.6, 3.0 Hz, 1 H), 3.38-3.30 (m, 3 H), 3.24-3.18 (m, 1 H), 3.02 (dd, J=12.6, 8.6 Hz, 1 H), 1.03 (d, J=6.4 Hz, 3 H). MS calculated for C₁₁H₁₄ClFN₂+H: 229, observed 229.

### Example 3.46

### Preparation of (R)-1-(5-Chloro-2-methyl-phenyl)-2-methyl-piperazine hydrochloride salt (Compound 46 HCl salt)

By the same general procedure as in Example 3.2, (R)-1-(5-Chloro-2-methyl-phenyl)-2-methyl-piperazine was obtained from 2-bromo-4-chlorotoluene as a light brown solid. ¹H NMR (400 MHz, CD₃OD) δ 7.24 (d, J=8.4 Hz, 1 H), 7.23 (d, J=2.4 Hz, 1 H), 7.12 (dd, J=8.0, 2.0 Hz, 1 H), 3.45-3.33 (m, 3 H), 3.30-3.26 (m, 1 H), 3.09-3.04 (appar dt, J=13.2 Hz, 1 H), 2.98-2.92 (m, 2 H), 2.30 (s, 3 H), 0.90 (d, J=6.4 Hz, 3 H). MS calculated for C₁₂H₁₇ClN₂+H: 225, observed 225.

### Example 3.47

### Preparation of (S)-1-(5-Chloro-2-methyl-phenyl)-2-methyl-piperazine trifluoroacetic acid salt (Compound 47 TFA salt)

By the same general procedure as in Example 3.2, (S)-1-(5-Chloro-2-methyl-phenyl)-2-methyl-piperazine was obtained from 2-bromo-4-chlorotoluene and (S)-4-N-boc-2-methyl-piperazine as a white solid. ¹H NMR (400 MHz, CD₃OD) δ 7.24 (d, J=8.0 Hz, 1 H), 7.21 (d, J=2.0 Hz, 1 H), 7.13 (dd, J=8.2, 2.2 Hz, 1 H), 3.45-3.32 (m, 3 H), 3.26 (dd, J=12.2, 3.4 Hz, 1 H), 3.06 (dt, J=13.3, 2.9 Hz, 1 H), 2.97-2.89 (m, 2 H), 2.30 (s, 3 H), 0.90 (d, J=6.4 Hz, 3 H). MS calculated for C₁₂H₁₇ClN₂+H: 225, observed 225.

### Example 3.48

### Preparation of (R,S) 1-(3-Chloro-4-fluoro-phenyl)-2-ethyl-piperazine trifluoroacetic acid salt (Compound 48 TFA salt)

By the same general procedure as in Example 3.2, 1-(3-chloro-4-fluoro-phenyl)-2-ethyl-piperazine was obtained from 4-bromo-2chloro-1-fluorobenzene and 4-N-boc-2-ethyl-piperazine as a white solid. ¹H NMR (400 MHz, CD₃OD) δ 7.15 (t, J=9.0 Hz, 1 H), 7.13 (d, J=3.6 Hz, 1 H), 6.98 (ddd, J=9.2, 3.8, 3.0 Hz, 1 H), 3.65 (sextet, J=4.4 Hz, 1 H), 3.39-3.32 (m, 3 H), 3.30-3.22 (m, 3 H), 1.61-1.51 (m, 2 H), 0.88 (t, J=7.4 Hz, 3 H). MS calculated for C₁₂H₁₆FClN₂+H: 243, observed 243.

### Example 3.49

### Preparation of (R,S) 1-(3-Chloro-phenyl)-2-ethyl-piperazine trifluoroacetic acid salt (Compound 49 TFA salt)

By the same general procedure as in Example 3.2, 1-(3-chloro-phenyl)-2-ethyl-piperazine was obtained from 1-bromo-3-chlorobenzene and 4-N-boc-2-ethyl-piperazine as a white solid. ¹H NMR (400 MHz, CD₃OD) δ 7.22 (t, J=8.2 Hz, 1 H), 6.99 (d, J=1.6 Hz, 1 H), 6.92-6.87 (m, 1 H), 3.86 (sextet, J=4.3 Hz, 1 H), 3.50 (dt, J=12.9, 2.9Hz, 1 H), 3.38-3.32 (m, 3 H), 3.26-3.18 (m, 2 H), 1.69-1.56 (m, 2 H), 0.90 (t, J=7.4 Hz, 3 H).

### Example 3.50

### Preparation of (R,S) 1-(4-Chloro-phenyl)-2ethyl-piperazine hydrochloride salt (Compound 50 HCl salt)

By the same general procedure as in Example 3.2, 1-(4-chloro-phenyl)-2-ethyl-piperazine was obtained from 1-bromo-4-chlorobenzene and 4-N-boc-2-ethyl-piperazine as a white solid. ¹H NMR (400 MHz, CD₃OD) δ 7.33-7.30 (m, 2 H), 7.17 (d, J=10.8 Hz, 2 H), 3.84 (sextet, J=4.6 Hz, 1 H), 3.58-3.53 (m, 1 H), 3.48-3.29 (m, 5 H), 1.62-1.48 (m, 2 H), 0.84 (t, J=7.4 Hz, 3 H).

### Example 3.51

### Preparation of (R,S) 1-(3,4-Difluoro-phenyl)-2-ethyl-piperazine hydrochloride salt (Compound 51 HCl salt)

By the same general procedure as in Example 3.2, 1-(3,4-difluoro-phenyl)-2-ethyl-piperazine was obtained from 1-bromo-3,4-difluorobenzene and 4-N-boc-2-ethyl-piperazine as a white solid. ¹H NMR (400 MHz, CD₃OD) δ 7.15 (dt, J=10.3, 9.1 Hz, 1 H), 6.98 (ddd, J=12.8, 7.0, 3.0 Hz, 1 H), 6.83-6.80 (m, 1 H), 3.69 (sextet, J=4.4 Hz, 1 H), 3.40-3.19 (m, 6 H), 1.60-1.47 (m, 2 H), 0.84 (t, J=7.4 Hz, 3 H).

### Example 3.52

### Preparation of (R,S)-1-(5-Chloro-2-fluoro-phenyl)-2-ethyl-piperazine hydrochloride salt (Compound 52 HCl salt)

By the same general procedure as in Example 3.2, (R)-1-(5-Chloro-2-fluoro-phenyl)-2-ethyl-piperazine was obtained from 2-bromo-4-chloro-1-fluorobenzene and 4-N-boc-2-ethyl-piperazine as a white solid. ¹H NMR (400 MHz, CD₃OD) δ 7.24-7.22 (m, 1 H), 7.17-7.09 (m, 2 H), 3.55 (qd, J=6.5, 3.2 Hz, 1 H), 3.45 (dd, J=12.8, 3.2 Hz, 1 H), 3.39-3.24 (m, 4 H), 3.17 (dd, J=12.8, 7.2 Hz, 1 H), 1.55 (quintet, J=7.2 Hz, 2 H), 0.85 (t, J=7.4 Hz, 3 H).

### Example 3.53

### Preparation of (R)-1-(2-Fluoro-5-trifluoromethyl-phenyl)-2-methyl-piperazine hydrochloride salt (Compound 53 HCl salt)

By the same general procedure as in Example 3.2, Compound 53 was obtained from the appropriate intermediates. ¹H NMR (400 MHz, CD₃OD) δ 7.55-7.52 (m, 2 H), 7.37-7.32 (m, 1 H), 3.72-3.64 (m, 1 H), 3.45 (dd, J=12.0, 4.0 Hz, 1 H), 3.40-3.35 (m, 3 H), 3.32-3.23 (m, 1 H), 3.04 (dd, J=12.4, 8.4, 1 H), 1.03 (d, J=4.0 Hz, 3 H). MS calculated for C₁₂H₁₄F₄N₂+H: 263, observed 263.

### Example 3.54

### Preparation of (S)-1-(2-Fluoro-5-trifluoromethyl-phenyl)-2-methyl-piperazine hydrochloride salt (Compound 54 HCl salt)

By the same general procedure as in Example 3.2, Compound 54 was obtained from the appropriate intermediates. ¹H NMR (400 MHz, CD₃OD) δ 7.55-7.52 (m, 2 H), 7.36-7.31 (m, 1H), 7.36-7.3 m, 1 H), 3.70-3.62 (m, 1 H), 3.43 (dd, J=12.8, 3.2 Hz, 1 H), 3.37-3.32(m, 3 H), 3.28-3.24 (m, 1H), 3.03 (dd, J=12.6, 8.6, Hz, 1 H), 1.02 (d, J=4.0 Hz, 3 H). MS calculated for C₁₂H₁₄F₄N₂+H: 263, observed 263.

### Example 3.55

### Preparation of (R)-1-(4-Chloro-2-fluoro-phenyl)-2-methyl-piperazine hydrochloride salt (Compound 55 HCl salt)

By the same general procedure as in Example 3.2, Compound 55 was obtained from the appropriate intermediates. ¹H NMR (400 MHz, CD₃OD) δ 7.26-7.24 (m, 1 H), 7.22-7.18 (m, 2 H), 3.60-3.52 (m, 1 H), 3.44-3.35 (m, 2 H), 3.33-3.21 (m, 3 H), 2.99 (dd, J=12.2, 9.4, Hz, 1 H), 1.00 (d, J=6.4 Hz, 3 H). MS calculated for C₁₁H₁₄ClFN₂+H: 229, observed 229.

### Example 3.56

### Preparation of (S)-1-(4-Chloro-2-fluoro-phenyl)-2-methyl-piperazine hydrochloride salt (Compound 56 HCl salt)

By the same general procedure as in Example 3.2, Compound 56 was obtained from the appropriate intermediates. ¹H NMR (400 MHz, CD₃OD) δ 7.26-7.24 (m, 1 H), 7.22-7.18 (m, 2 H), 3.60-3.52 (m, 1 H), 3.44-3.35 (m, 2 H), 3.33-3.21 (m, 3 H), 2.99 (dd, J=12.2, 9.4, Hz, 1 H), 1.00 (d, J=6.4 Hz, 3 H). MS calculated for C₁₁H₁₄ClFN₂+H: 229, observed 229.

### Example 3.57

### Preparation of (R)-1-(3-Chloro-5-fluoro-phenyl)-2-methyl-piperazine hydrochloride salt (Compound 57 HCl salt)

By the same general procedure as in Example 3.2, Compound 57 was obtained from the appropriate intermediates. ¹H NMR (400 MHz, CD₃OD) δ 6.86 (s, 1 H), 6.76-6.69 (m, 2 H), 4.23-4.16 (m, 1 H), 3.58-3.53 (m, 1 H), 3.44-3.25 (m, 5 H), 1.16 (d, J=6.8 Hz, 3 H). MS calculated for C₁₁H₁₄ClFN₂+H: 229, observed 229.

### Example 3.58

### Preparation of (S)-1-(3-Chloro-5-fluoro-phenyl)-2-methyl-piperazine hydrochloride salt (Compound 58 HCl salt)

By the same general procedure as in Example 3.2, Compound 58 was obtained from the appropriate intermediates. ¹H NMR (400 MHz, CD₃OD) δ 6.85 (s, 1 H), 6.75-6.69 (m, 2 H), 4.22-4.15 (m, 1 H), 3.57-3.50 (m, 1 H), 3.43-3.37 (m, 2 H), 3.28-3.19 (m, 3 H), 1.16 (d, J=6.8 Hz, 3 H). MS calculated for C₁₁H₁₄ClFN₂+H: 229, observed 229.

### Example 3.59

### Preparation of (R)-1-(2-Fluoro-phenyl)-2-methyl-piperazine hydrochloride salt (Compound 59 HCl salt)

By the same general procedure as in Example 3.2, Compound 59 was obtained from the appropriate intermediates. ¹H NMR (400 MHz, CD₃OD) δ 7.26-7.09 (m, 4 H), 3.60-3.52 (m, 1 H), 3.43-3.32 (m, 3 H), 3.28-3.22 (m, 2 H), 2.97 (dd, J=12.2, 9.0 Hz, 1 H), 0.99 (d, J=10.4 Hz, 3 H). MS calculated for C₁₁H₁₅FN₂+H: 195, observed 195.

### Example 3.60

### Preparation of (S)-1-(2-Fluoro-phenyl)-2-methyl-piperazine hydrochloride salt (Compound 60 HCl salt)

By the same general procedure as in Example 3.2, Compound 60 was obtained from the appropriate intermediates. ¹H NMR (400 MHz, CD₃OD) δ 7.26-7.09 (m, 4 H), 3.60-3.52 (m, 1 H), 3.43-3.32 (m, 3 H), 3.28-3.22 (m, 2 H), 2.97 (dd, J=12.2, 9.0 Hz, 1 H), 0.99 (d, J=10.4 Hz, 3 H). MS calculated for C₁₁H₁₅FN₂+H: 195, observed 195.

### Example 3.61

### Preparation of (R)-1-(3-Fluoro-phenyl)-2-methyl-piperazine hydrochloride salt (Compound 61 HCl salt)

By the same general procedure as in Example 3.2, Compound 61 was obtained from the appropriate intermediates. ¹H NMR (400 MHz, CD₃OD) δ 7.39 (q, J=7.7 Hz, 1 H), 7.07-7.02 (m, 2 H), 6.86 (td, J=8.2, 2.0 Hz, 1 H), 4.18-4.10 (m, 1 H), 3.66-3.35 (m, 6 H), 1.15 (d, J=6.8 Hz, 3 H). MS calculated for C₁₁H₁₅1FN₂+H: 195, observed 195.

### Example 3.62

### Preparation of (S)-1-(3-Fluoro-phenyl)-2-methyl-piperazine hydrochloride salt (Compound 62 HCl salt)

By the same general procedure as in Example 3.2, Compound 62 was obtained from the appropriate intermediates. ¹H NMR (400 MHz, CD₃OD) δ 7.29 (td, J=8.2, 6.9 Hz, 1 H), 6.84 (dd, J=7.8, 1.4 Hz, 1 H), 6.78 (dt, J=11.9, 2.3 Hz, 1 H), 6.71-6.67 (m, 1 H), 1.11 (d, J=6.8 Hz, 3 H). MS calculated for C₁₁H₁₅FN₂+H: 195, observed 195.

### Example 3.63

### Preparation of (R)-1-(2-Fluoro-4-trifluoromethyl-phenyl)-2-methyl-piperazine hydrochloride salt (Compound 63 HCl salt)

By the same general procedure as in Example 3.2, Compound 63 was obtained from the appropriate intermediates. ¹H NMR (400 MHz, CD₃OD) δ 7.50-7.45 (m, 2 H), 7.37 (t, J=8.4 Hz, 1 H), 3.86-3.78 (m, 1 H), 3.48-3.41 (m, 2 H), 3.38-3.36 (m, 2 H), 3.29-3.24 (m, 1 H), 3.11 (dd, J=12.8, 7.2 Hz, 1 H), 1.09 (d, J=6.4 Hz, 3 H). MS calculated for C₁₂H₁₄1F₄N₂+H: 263, observed 263.

### Example 3.64

### Preparation of (S)-1-(2-Fluoro-4-trifluoromethyl-phenyl)-2-methyl-piperazine hydrochloride salt (Compound 64 HCl salt)

By the same general procedure as in Example 3.2, Compound 64 was obtained from the appropriate intermediates. ¹H NMR (400 MHz, CD₃OD) δ 7.49-7.45 (m, 2 H), 7.37 (t, J=8.4 Hz, 1 H), 3.86-3.78 (m, 1 H), 3.48-3.41 (m, 2 H), 3.38-3.36 (m, 2 H), 3.28-3.24 (m, 1 H), 3.11 (dd, J=12.8, 7.2 Hz, 1 H), 1.08 (d, J=6.4 Hz, 3 H). MS calculated for C₁₂H₁₄F₄N₂+H: 263, observed 263.

### Example 3.65

### Preparation of (R)-1-(2-Chloro-3-fluoro-phenyl)-2-methyl-piperazine hydrochloride salt (Compound 65 HCl salt)

By the same general procedure as in Example 3.2, Compound 65 was obtained from the appropriate intermediates. ¹H NMR (400 MHz, CD₃OD) δ 7.47 (dd, J=8.8, 5.6 Hz, 1 H), 7.12 (dd, J=10.0, 2.8 Hz, 1 H), 7.0-6.95 (m, 1 H), 3.62-3.54 (m, 1 H), 3.47-3.37 (m, 2 H), 3.35-3.27 (m, 2 H), 3.03-2.95 (m, 2 H), 0.99 (d, J=6.0 Hz, 3 H). MS calculated for C₁₁H₁₄ClFN₂+H: 229, observed 229.

### Example 3.66

### Preparation of (S)-1-(2-Chloro-3-fluoro-phenyl)-2-methyl-piperazine hydrochloride salt (Compound 66 HCl salt)

By the same general procedure as in Example 3.2, Compound 66 was obtained from the appropriate intermediates. ¹H NMR (400 MHz, CD₃OD) δ 7.47 (dd, J=8.8, 5.6 Hz, 1 H), 7.11 (dd, J=10.0, 2.8 Hz, 1 H), 7.0-6.95 (m, 1 H), 3.59-3.53 (m, 1 H), 3.47-3.32 (m, 2 H), 3.29-3.28 (m, 2 H), 3.03-2.94 (m, 2 H), 1.00 (d, J=6.0 Hz, 3 H). MS calculated for C₁₁H₁₄ClFN₂+H: 229, observed 229.

### Example 3.67

### Preparation of (R)-1-(2-Fluoro-5-methyl-phenyl)-2-methyl-piperazine hydrochloride salt (Compound 67 HCl salt)

By the same general procedure as in Example 3.2, Compound 67 was obtained from the appropriate intermediates. ¹H NMR (400 MHz, CD₃OD) δ 7.04 (d, J=8.4 Hz, 1 H), 7.00-6.95 (m, 2 H), 3.58-3.50 (m, 1 H), 3.42-3.35 (m, 2 H), 3.31-3.29 (m, 1 H), 3.27-3.22 (m, 2 H), 2.96 (dd, J=12.2, 9.4 Hz, 1 H), 2.30 (s, 3 H), 0.97 (d, J=6.4 Hz, 3 H). MS calculated for C₁₂H₁₇FN₂+H: 209, observed 209.

### Example 3.68

### Preparation of (S)-1-(2-Fluoro-5-methyl-phenyl)-2-methyl-piperazine hydrochloride salt (Compound 68 HCl salt)

By the same general procedure as in Example 3.2, Compound 68 was obtained from the appropriate intermediates. ¹H NMR (400 MHz, CD₃OD) δ 7.04 (d, J=8.4 Hz, 1 H), 7.00-6.97 (m, 2 H), 3.58-3.50 (m, 1 H), 3.42-3.34 (m, 2 H), 3.29-3.22 (m, 3 H), 2.96 (dd, J=12.2, 9.4 Hz, 1 H), 2.30 (s, 3 H), 0.97 (d, J=6.4 Hz, 3 H). MS calculated for C₁₂H₁₇FN₂+H: 209, observed 209.

### Example 3.69

### Preparation of (R)-1-(4-Fluoro-biphenyl-3-yl)-2-methyl-piperazine hydrochloride salt (Compound 69 HCl salt)

By the same general procedure as in Example 3.2, Compound 69 was obtained from the appropriate intermediates. ¹H NMR (400 MHz, CD₃OD) δ 7.74 (s, 1 H), 7.71-7.67 (m, 2 H), 7.60-7.56 (m, 2 H), 7.49-7.47 (m, 1 H), 7.23-7.19 (m, 2 H), 4.27-4.19 (m, 1 H), 3.88-3.80 (m, 1 H), 3.76-3.67 (m, 3 H), 3.62-3.56 m (1 H), 3.51 (dd, J=13.2, 9.2 Hz, 1 H), 1.20 (d, J=6.4 Hz, 3 H). MS calculated for C₁₇H₁₉FN₂+H: 271, observed 271.

### Example 3.70

### Preparation of (S)-1-(4-Fluoro-biphenyl-3-yl)-2-methyl-piperazine hydrochloride salt (Compound 70 HCl salt)

By the same general procedure as in Example 3.2, Compound 70 was obtained from the appropriate intermediates. ¹H NMR (400 MHz, CD₃OD) δ 7.74 (s, 1 H), 7.71-7.67 (m, 2 H), 7.60-7.56 (m, 2 H), 7.49-7.47 (m, 1 H), 7.23-7.19 (m, 2 H), 4.27-4.19 (m, 1 H), 3.88-3.80 (m, 1 H), 3.76-3.67 (m, 3 H), 3.62-3.56 m (1 H), 3.51 (dd, J=13.2, 9.2 Hz, 1 H), 1.20 (d, J=6.4 Hz, 3 H). MS calculated for C₁₇H₁₉FN₂+H: 271, observed 271.

### Example 3.71

### Preparation of (R)-1-(2,5-Difluoro-4-methoxy-phenyl)-2-methyl-piperazine hydrochloride salt (Compound 71 HCl salt)

By the same general procedure as in Example 3.2, Compound 71 was obtained from the appropriate intermediates. ¹H NMR (400 MHz, CD₃OD) δ 7.05 (dd, J=12.0, 7.2 Hz, 1 H), 6.96 (dd, J=12.8, 8.0 Hz, 1 H), 3.85 (s, 3 H), 3.41-3.36 (m, 3 H), 3.30-3.19 (m, 3 H), 2.91 (dd, J=12.8, 10.4 Hz, 1 H), 0.95 (d, J=6.0 Hz, 3 H). MS calculated for C₁₂H₁₆F₂N₂+H: 243, observed 243.

### Example 3.72

### Preparation of (S)-1-(2,5-Difluoro-4-methoxy-phenyl)-2-methyl-piperazine hydrochloride salt (Compound 72 HCl salt)

By the same general procedure as in Example 3.2, Compound 72 was obtained from the appropriate intermediates. ¹H NMR (400 MHz, CD₃OD) δ 7.05 (dd, J=11.8, 7.4 Hz, 1 H), 6.96 (dd, J=12.8, 8.0 Hz, 1 H), 3.85 (s, 3 H), 3.39-3.36 (m, 3 H), 3.30-3.19 (m, 3 H), 2.91 (dd, J=12.8, 10.4 Hz, 1 H), 0.95 (d, J=6.0 Hz, 3 H). MS calculated for C₁₂H₁₆F₂N₂+H: 243, observed 243.

### Example 3.73

### Preparation of (R)-1-(2-Fluoro-4-methyl-phenyl)-2-methyl-piperazine hydrochloride salt (Compound 73 HCl salt)

By the same general procedure as in Example 3.2, Compound 73 was obtained from the appropriate intermediates. ¹H NMR (400 MHz, CD₃OD) δ 7.11 (t, J=8.4 Hz, 1 H), 6.98-6.93 (m, 2 H), 3.53-3.45 (m, 1 H), 3.40-3.34 (m, 2 H), 3.30-3.19 (m, 3 H), 2.93 (dd, J=12.4, 9.6 Hz, 1 H), 2.32 (s, 3 H), 0.95 (d, J=6.4 Hz, 3 H). MS calculated for C₁₂H₁₇FN₂+H: 209, observed 209.

### Example 3.74

### Preparation of (S)-1-(2-Fluoro-4-methyl-phenyl)-2-methyl-piperazine hydrochloride salt (Compound 74 HCl salt)

By the same general procedure as in Example 3.2, Compound 74 was obtained from the appropriate intermediates. ¹H NMR (400 MHz, CD₃OD) δ 7.04 (d, J=8.4 Hz, 1 H), 7.00-6.95 (m, 2 H), 3.55-3.52 (m, 1 H), 3.42-3.34 (m, 2 H), 3.29-3.22 (m, 3 H), 2.93 (dd, J=12.4, 9.2 Hz, 1 H), 2.31 (s, 3 H), 0.95 (d, J=6.4 Hz, 3 H). MS calculated for C₁₂H₁₇FN₂+H: 209, observed 209.

### Example 3.75

### Preparation of (R)-1-(2-Chloro-5-fluoro-phenyl)-2-methyl-piperazine hydrochloride salt (Compound 75 HCl salt)

By the same general procedure as in Example 3.2, Compound 75 was obtained from the appropriate intermediates. ¹H NMR (400 MHz, CD₃OD) δ 7.47 (dd, J=8.8, 6.0 Hz, 1 H), 7.15 (dd, J=9.8, 3.0 Hz, 1 H), 6.98 (ddd, J=8.8, 7.6, 2.8 Hz, 1 H), 3.60-3.52 (m, 1 H), 3.46-3.42 (m, 1 H), 3.41-3.33 (m, 2 H), 3.30-3.27 (m, 1 H), 3.03-2.95 (m, 2 H), 1.00 (d, J=6.4 Hz, 3 H). MS calculated for C₁₁H₁₄ClFN₂+H: 229, observed 229.

### Example 3.76

### Preparation of (S)-1-(2-Chloro-5-fluoro-phenyl)-2-methyl-piperazine hydrochloride salt (Compound 76 HCl salt)

By the same general procedure as in Example 3.2, Compound 76 was obtained from the appropriate intermediates. ¹H NMR (400 MHz, CD₃OD) δ 7.47 (dd, J=8.8, 6.0 Hz, 1 H), 7.15 (dd, J=9.8 Hz, 3.0 Hz, 1 H), 6.98 (ddd, J=8.8, 7.6, 2.8 Hz, 1 H), 3.60-3.52 (m, 1 H), 3.46-3.42 (m, 1 H), 3.41-3.33 (m 2 H), 3.30-3.27 (m, 1 H), 3.03-2.95 (m, 2 H), 1.00 (d, J=6.4 Hz, 3 H). MS calculated for C₁₁H₁₄ClFN₂+H: 229, observed 229.

### Example 3.77

### Preparation of (R)-1-(2-Chloro-4-fluoro-phenyl)-2-methyl-piperazine hydrochloride salt (Compound 77 HCl salt)

By the same general procedure as in Example 3.2, Compound 77 was obtained from the appropriate intermediates. ¹H NMR (400 MHz, CD₃OD) δ 7.34 (dd, J=8.8, 5.6 Hz, 1 H), 7.29 (dd, J=8.4, 2.8 Hz, 1 H), 7.12 (ddd, J=9.0, 7.8, 2.8 Hz, 1 H), 3.52-3.37 (m, 3 H), 3.33-3.26 (m, 1 H), 3.20 (dt, J=12.9, 3.1 Hz, 1 H), 3.04-2.94 (m, 2 H), 0.94 (d, J=6.4 Hz, 3 H). MS calculated for C₁₁H₁₄ClFN₂+H: 229, observed 229.

### Example 3.78

### Preparation of (S)-1-(2-Chloro-4-fluoro-phenyl)-2-methyl-piperazine hydrochloride salt (Compound 78 HCl salt)

By the same general procedure as in Example 3.2, Compound 78 was obtained from the appropriate intermediates. ¹H NMR (400 MHz, CD₃OD) δ 7.34 (dd, J=8.8, 5.6 Hz, 1 H), 7.29 (dd, J=8.4, 2.8 Hz, 1 H), 7.12 (ddd, J=9.0, 7.8, 2.8 Hz, 1 H), 3.52-3.37 (m, 3 H), 3.33-3.26 (m, 1 H), 3.20 (dt, J=12.9, 3.1 Hz, 1 H), 3.04-2.94 (m, 2 H), 0.94 (d, J=6.4 Hz, 3 H). MS calculated for C₁₁H₁₄ClFN₂+H: 229, observed 229.

### Example 3.79

### Preparation of (R)-1-(2,4-Dichloro-phenyl)-2-methyl-piperazine hydrochloride salt (Compound 79 HCl salt)

By the same general procedure as in Example 3.2, Compound 79 was obtained from the appropriate intermediates. ¹H NMR (400 MHz, CD₃OD) δ 7.52 (d, J=2.4 Hz, 1 H), 7.36 (dd, J=8.4, 2.4 Hz, 1 H), 7.28 (d, J=8.4 Hz, 1 H), 3.53-3.22 (m, 5 H), 2.99-2.93 (m, 2 H), 0.96 (d, J=6.4 Hz, 3 H). MS calculated for C₁₁H₁₄Cl₂N₂+H: 245, observed 245.

### Example 3.80

### Preparation of (S)-1-(2,4-Dichloro-phenyl)-2-methyl-piperazine hydrochloride salt (Compound 80 HCl salt)

By the same general procedure as in Example 3.2, Compound 80 was obtained from the appropriate intermediates. ¹H NMR (400 MHz, CD₃OD) δ 7.52 (d, J=2.4 Hz, 1 H), 7.36 (dd, J=8.4, 2.4 Hz, 1 H), 7.28 (d, J=8.4 Hz, 1 H), 3.53-3.22 (m, 5 H), 2.99-2.93 (m, 2 H), 0.96 (d, J=6.4 Hz, 3 H). MS calculated for C₁₁H₁₄Cl₂N₂+H: 245, observed 245.

### Example 3.81

### Preparation of (R)-1-(2,5-Dichloro-phenyl)-2-methyl-piperazine hydrochloride salt (Compound 81 HCl salt)

By the same general procedure as in Example 3.2, Compound 81 was obtained from the appropriate intermediates. ¹H NMR (400 MHz, CD₃OD) δ 7.45 (d, J=8.8 Hz, 1 H), 7.33 (d, J=2.4 Hz, 1 H), 7.22 (dd, J=8.4, 2.4 Hz, 1 H), 3.60-3.55 (m, 1 H), 3.46-3.37 (m, 2 H), 3.34-3.26 (m, 2 H), 3.02-2.96 (m, 2 H), 0.99 (d, J=6.4 Hz, 3 H). MS calculated for C₁₁H₁₄Cl₂N₂+H: 245, observed 245.

### Example 3.82

### Preparation of (S)-1-(2,5-Dichloro-phenyl)-2-methyl-piperazine hydrochloride salt (Compound 82 HCl salt)

By the same general procedure as in Example 3.2, Compound 82 was obtained from the appropriate intermediates. ¹H NMR (400 MHz, CD₃OD) δ 7.46 (d, J=8.8 Hz, 1 H), 7.33 (d, J=2.4 Hz, 1 H), 7.22 (dd, J=8.4, 2.4 Hz, 1 H), 3.60-3.53 (m, 1 H), 3.46-3.36 (m, 2 H), 3.35-3.26 (m, 2 H), 3.03-2.97 (m, 2 H), 0.99 (d, J=6.0 Hz, 3 H). MS calculated for C₁₁H₁₄Cl₂N₂+H: 245, observed 245.

### Example 3.83

### Preparation of (R)-1-(3,5-Bis-trifluoromethyl-phenyl)-2-methyl-piperazine hydrochloride salt (Compound 83 HCl salt)

By the same general procedure as in Example 3.2, Compound 83 was obtained from the appropriate intermediates. ¹H NMR (400 MHz, CD₃OD) δ 7.51 (s, 2 H), 7.42 (s, 1 H), 4.42-4.35 (m, 1 H), 3.70 (dt, J=13.3, 3.5 Hz, 1 H), 3.50-3.41 (m, 2 H), 3.39-3.33 (m, 2 H), 3.27-3.23 (m, 1 H), 1.20 (d, J=6.8 Hz, 3 H). MS calculated for C₁₃H₁₄F₆N₂+H: 313, observed 313.

### Example 3.84

### Preparation of (S)-1-(3,5-Bis-trifluoromethyl-phenyl)-2-methyl-piperazine hydrochloride salt (Compound 84 HCl salt)

By the same general procedure as in Example 3.2, Compound 84 was obtained from the appropriate intermediates. ¹H NMR (400 MHz, CD₃OD) δ 7.51 (s, 2 H), 7.42 (s, 1 H), 4.42-4.35 (m, 1 H), 3.70 (dt, J=13.3, 3.5 Hz, 1 H), 3.50-3.41 (m, 2 H), 3.39-3.33 (m, 2 H), 3.27-3.23 (m, 1 H), 1.20 (d, J=6.8 Hz, 3 H). MS calculated for C₁₃H₁₄F₆N₂+H: 313, observed 313.

### Example 3.85

### Preparation of (R)-1-(4-Fluoro-2-methyl-phenyl)-2-methyl-piperazine hydrochloride salt (Compound 85 HCl salt)

By the same general procedure as in Example 3.2, Compound 85 was obtained from the appropriate intermediates. ¹H NMR (400 MHz, DMSO-d6) δ 9.00 (br s, 1 H), 8.94 (br, 1 H), 7.17 (dd, J=8.8, 5.6Hz, 1 H), 7.08 (dd, J=9.8, 3.0 Hz, 1 H), 7.03 (td, J=8.5, 2.9 Hz, 1 H), 3.36 (d, J=12.4 Hz, 1 H), 3.30-3.24 (m, 2 H), 3.10 (appar q, J=10.9 Hz, 1 H), 2.93-2.90 (m, 2 H), 2.54 (s, 3 H), 0.76 (d, J=6.4 Hz, 3 H). MS calculated for C₁₂H₁₇FN₂+H: 209, observed 209.

### Example 3.86

### Preparation of (S)-1-(4-Fluoro-2-methyl-phenyl)2-methyl-piperazine hydrochloride salt (Compound 86 HCl salt)

By the same general procedure as in Example 3.2, Compound 86 was obtained from the appropriate intermediates. ¹H NMR (400 MHz, CD₃OD) δ 7.21 (dd, J=8.6, 5.4 Hz, 1 H), 6.99 (dd, J=9.4, 3.0 Hz, 1 H), 6.94 (td, J=8.2, 2.9 Hz, 1 H), 3.44-3.36 (m, 3 H), 3.30-3.26 (m, 1 H), 3.04-2.89 (m, 3 H), 2,33 (s, 3 H), 0.87 (d, J=6.4 Hz, 3 H). MS calculated for C₁₂H₁₇FN₂+H: 209, observed 209.

### Example 3.87

### Preparation of (R)-1-(2-Chloro-phenyl)-2-methyl-piperazine hydrochloride salt (Compound 87 HCl salt)

By the same general procedure as in Example 3.2, Compound 87 was obtained from the appropriate intermediates. ¹H NMR (400 MHz, CD₃OD) δ 7.45 (dd, J=7.8, 1.4 Hz, 1 H), 7.36-7.29 (m, 2 H), 7.20-7.16 (m, 1 H), 3.61-3.53 (m, 1 H), 3.46-3.37 (m, 2 H), 3.34-3.30 (m, 1 H), 3.27-3.23 (m, 1 H), 3.05-2.95 (m, 2 H), 0.96 (d, J=6.0 Hz, 3 H). MS calculated for C₁₁H₁₅ClN₂+H: 211, observed 211.

### Example 3.88

### Preparation of (S)-1-(2-Chloro-phenyl)-2-methyl-piperazine hydrochloride salt (Compound 88 HCl salt)

By the same general procedure as in Example 3.2, Compound 88 was obtained from the appropriate intermediates. ¹H NMR (400 MHz, CD₃OD) δ 7.45 (dd, J=7.8, 1.4 Hz, 1 H), 7.36-7.29 (m, 2 H), 7.20-7.16 (m, 1 H), 3.61-3.53 (m, 1 H), 3.46-3.41 (m, 2 H), 3.34-3.24 (m, 2 H), 3.05-2.95 (m, 2 H), 0.96 (d, J=6.0 Hz, 3 H). MS calculated for C₁₁H₁₅ClN₂+H: 211, observed 211.

### Example 3.89

### Preparation of (R)-1-(2,3-Dichloro-phenyl)-2-methyl-piperazine hydrochloride salt (Compound 89 HCl salt)

By the same general procedure as in Example 3.2, Compound 89 was obtained from the appropriate intermediates. ¹H NMR (400 MHz, CD₃OD) δ 7.39 (dd, J=7.6, 2.0 Hz, 1 H), 7.33 (t, J=7.8 Hz, 1 H), 7.29 (dd, J=8.0, 2.0 Hz, 1 H), 3.65-3.57 (m, 1 H), 3.48-3.39 (m, 2 H), 3.35-3.25 (m, 2 H), 3.06-2.97 (m, 2 H), 0.97 (d, J=6.4 Hz, 3 H). MS calculated for C₁₁H₁₄Cl₂N₂+H: 245, observed 245.

### Example 3.90

### Preparation of (S)-1-(2,3-Dichloro-phenyl)-2-methyl-piperazine hydrochloride salt (Compound 90 HCl salt)

By the same general procedure as in Example 3.2, Compound 90 was obtained from the appropriate intermediates. ¹H NMR (400 MHz, CD₃OD) δ 7.40 (dd, J=7.6, 1.6 Hz, 1 H), 7.33 (t, J=7.8 Hz, 1 H), 7.29 (dd, J=7.8, 1.8 Hz, 1 H), 3.64-3.56 (m, 1 H), 3.48-3.38 (m, 2 H), 3.39-3.25 (m, 2 H), 3.05-2.97 (m, 2 H), 0.97 (d, J=6.4 Hz, 3 H). MS calculated for C₁₁H₁₄Cl₂N₂+H: 245, observed 245.

### Example 3.91

### Preparation of (R)-1-(2,6-Dichloro-phenyl)-2-methyl-piperazine hydrochloride salt (Compound 91 HCl salt)

By the same general procedure as in Example 3.2, Compound 91 was obtained from the appropriate intermediates. ¹H NMR (400 MHz, CD₃OD) δ 7.45 (d, J=8.8 Hz, 1 H), 7.33 (d, J=2.4 Hz, 1 H), 7.22 (dd, J=8.4, 2.4 Hz, 1 H), 3.59-3.55 (m, 1 H), 3.47-3.37 (m, 2 H), 3.34-3.26 (m, 2 H), 3.03-2.96 (m, 2 H), 0.99 (d, J=6.4 Hz, 3 H). MS calculated for C₁₁H₁₄Cl₂N₂+H: 245, observed 245.

### Example 3.92

### Preparation of (S)-1-(2,6-Dichloro-phenyl)-2-methyl-piperazine hydrochloride salt (Compound 92 HCl salt)

By the same general procedure as in Example 3.2, Compound 92 was obtained from the appropriate intermediates. ¹H NMR (400 MHz, CD₃OD) δ 7.45 (d, J=8.8 Hz, 1 H), 7.33 (d, J=2.4 Hz, 1 H), 7.21 (dd, J=8.4, 2.4 Hz, 1 H), 3.60-3.56 (m, 1 H), 3.47-3.37 (m, 2 H), 3.34-3.26 (m, 2 H), 3.03-2.97 (m, 2 H), 0.98 (d, J=6.0 Hz, 3 H). MS calculated for C₁₁H₁₄Cl₂N₂+H: 245, observed 245.

### Example 3.93

### Preparation of (R)-1-(2-Chloro-5-trifluoromethyl-phenyl)-2-methyl-piperazine hydrochloride salt (Compound 93 HCl salt)

By the same general procedure as in Example 3.2, Compound 93 was obtained from the appropriate intermediates. ¹H NMR (400 MHz, CD₃OD) δ 7.69 (d, J=8.4 Hz, 1 H), 7.58 (d, J=1.6 Hz, 1 H), 7.51 (dd, J=8.2, 1.4 Hz, 1 H), 3.69-3.61 (m, 1 H), 3.49-3.43 (m, 1 H), 3.42-3.30 (m, 3 H), 3.06-2.99 (m, 2 H), 0.98 (d, J=6.4 Hz, 3 H). MS calculated for C₁₂H₁₄ClF₃N₂+H: 279, observed 279.

### Example 3.94

### Preparation of (S)-1-(2-Chloro-5-trifluoromethyl-phenyl)-2-methyl-piperazine hydrochloride salt (Compound 94 HCl salt)

By the same general procedure as in Example 3.2, Compound 94 was obtained from the appropriate intermediates. ¹H NMR (400 MHz, CD₃OD) δ 7.68 (d, J=8.4 Hz, 1 H), 7.58 (d, J=2.0 Hz, 1 H), 7.51 (dd, J=8.4, 2.0 Hz, 1 H), 3.69-3.61 (m, 1 H), 3.49-3.43 (m, 1 H), 3.42-3.30 (m, 3 H), 3.05-3.00 (m, 2 H), 0.98 (d, J=6.0 Hz, 3 H). MS calculated for C₁₂H₁₄ClF₃N₂+H: 279, observed 279.

### Example 3.95

### Preparation of (R)-2-Methyl-1-(3-trifluoromethyl-phenyl)-piperazine hydrochloride salt (Compound 95 HCl salt)

By the same general procedure as in Example 3.2, Compound 95 was obtained from the appropriate intermediates. ¹H NMR (400 MHz, CD₃OD) δ 7.49 (t, J=8.2 Hz, 1 H), 7.31-7.24 (m, 3 H), 4.12-4.07 (m, 1 H), 3.51-3.41 (m, 3 H), 3.34-3.23 (m, 3 H), 1.11 (d, J=6.4 Hz, 3 H). MS calculated for C₁₂H₁₅F₃N₂+H: 245, observed 245.

### Example 3.96

### Preparation of (S)-2-Methyl-1-(3-trifluoromethyl-phenyl)-piperazine hydrochloride salt (Compound 96 HCl salt)

By the same general procedure as in Example 3.2, Compound 96 was obtained from the appropriate intermediates. ¹H NMR (400 MHz, CD₃OD) δ 7.49 (t, J=8.8 Hz, 1 H), 7.30-7.29 (m, 2 H), 7.25 (d, J=8.0 Hz, 1 H), 4.12-4.07 (m, 1 H), 3.50-3.40 (m, 3 H), 3.34-3.23 (m, 3 H), 1.11 (d, J=6.8 Hz, 3 H). MS calculated for C₁₂H₁₅F₃N₂+H: 245, observed 245.

### Example 3.97

### Preparation of (R)-2-Methyl-1-(4-trifluoromethyl-phenyl)-piperazine hydrochloride salt (Compound 97 HCl salt)

By the same general procedure as in Example 3.2, Compound 97 was obtained from the appropriate intermediates. ¹H NMR (400 MHz, CD₃OD) δ 7.55 (d, J=8.8 Hz, 2 H), 7.12 (d, J=8.4 Hz, 2 H), 4.35-4.28 (m, 1 H), 3.66 (dt, J=12.9, 3.1 Hz, 1 H), 3.47-3.40 (m, 2 H), 3.36-3.21 (m, 3 H), 1.18 (d, J=7.2 Hz, 3 H). MS calculated for C₁₂H₁₅F₃N₂+H: 245, observed 245.

### Example 3.98

### Preparation of (S)-2-Methyl-1-(4-trifluoromethyl-phenyl)-piperazine hydrochloride salt (Compound 98 HCl salt)

By the same general procedure as in Example 3.2, Compound 98 was obtained from the appropriate intermediates. ¹H NMR (400 MHz, CD₃OD) δ 7.55 (d, J=8.8 Hz, 1 H), 7.12 (d, J=8.4 Hz, 1 H), 4.32-4.31 (m, 1 H), 3.66 (dt, J=12.9, 3.1 Hz, 1 H), 3.47-3.40 (m, 2 H), 3.36-3.24 (m, 3 H), 1.18 (d, J=7.2 Hz, 3 H). MS calculated for C₁₂H₁₅F₃N₂+H: 245, observed 245.

### Example 3.99

### Preparation of (R)-1-(2-Fluoro-3-trifluoromethyl-phenyl)-2-methyl-piperazine hydrochloride salt (Compound 99 HCl salt)

By the same general procedure as in Example 3.2, Compound 99 was obtained from the appropriate intermediates. ¹H NMR (400 MHz, DMSO-d6) δ 9.53 (br s, 1 H), 9.30 (br s, 1 H), 7.56 (t, J=7.6 Hz, 1 H), 7.50 (t, J=7.0 Hz, 1 H), 7.38 (t, J=7.8 Hz, 1 H), 3.69-3.65 (m, 1 H), 3.35-3.12 (m, 5 H), 2.97-2.91 (m, 1 H), 0.99 (d, J=6.8 Hz, 3 H). MS calculated for C₁₂H₁₄F₄N₂+H: 263, observed 263.

### Example 3.100

### Preparation of (S)-1-(2-Fluoro-3-trifluoromethyl-phenyl)-2-methyl-piperazine hydrochloride salt (Compound 100 HCl salt)

By the same general procedure as in Example 3.2, Compound 100 was obtained from the appropriate intermediates. ¹H NMR (400 MHz, CD₃OD) δ 7.56-7.49 (m, 2 H), 7.34 (t, J=8.2 Hz, 1 H), 3.65-3.58 (m, 1 H), 3.47-3.43 (m, 1 H), 3.37-3.23 (m, 4 H), 3.04 (dd, J=12.4, 8.8 Hz, 1 H), 1.02 (d, J=6.4 Hz, 3 H). MS calculated for C₁₂H₁₄F₄N₂+H: 263, observed 263.

### Example 3.101

### Preparation of (R)-1-(3-Fluoro-5-trifluoromethyl-phenyl)-2-methyl-piperazine hydrochloride salt (Compound 101 HCl salt)

By the same general procedure as in Example 3.2, Compound 101 was obtained from the appropriate intermediates. ¹H NMR (400 MHz, CD₃OD) δ 7.08 (s, 1 H), 7.03 (dt, J=11.9, 2.0 Hz, 1 H), 6.92 (d, J=8.4 Hz, 1 H), 4.32-4.25 (m, 1 H), 3.63 (dt, J=12.8, 2.9 Hz, 1 H), 3.46-3.40 (m, 2 H), 3.35-3.21 (m, 3 H), 1.18 (d, J=6.8 Hz, 3 H). MS calculated for C₁₂H₁₄F₄N₂+H: 263, observed 263.

### Example 3.102

### Preparation of (S)-1-(3-Fluoro-5-trifluoromethyl-phenyl)-2-methyl-piperazine hydrochloride salt (Compound 102 HCl salt)

By the same general procedure as in Example 3.2, Compound 102 was obtained from the appropriate intermediates. ¹H NMR (400 MHz, CD₃OD) δ 7.08 (s, 1 H), 7.03 (dt, J=11.7, 2.1 Hz, 1 H), 6.92 (d, J=8.0 Hz, 1 H), 4.32-4.26 (m, 1 H), 3.63 (dt, J=13.1, 3.2 Hz, 1 H), 3.46-3.40 (m, 2 H), 3.36-3.21 (m, 3 H), 1.19 (d, J=6.8 Hz, 3 H). MS calculated for C₁₂H₁₄F₄N₂+H: 263, observed 263.

### Example 3.103

### Preparation of (R)-1-(4-Chloro-3-trifluoromethyl-phenyl)-2-methyl-piperazine hydrochloride salt (Compound 103 HCl salt)

By the same general procedure as in Example 3.2, Compound 103 was obtained from the appropriate intermediates. ¹H NMR (400 MHz, CD₃OD) δ 7.51 (d, J=9.2 Hz, 1 H), 7.38 (d, J=2.8 Hz, 1 H), 7.28 (dd, J=8.6, 2.6 Hz, 1 H), 4.18-4.11 (m, 1 H), 3.55-3.50 (m, 1 H), 3.48-3.42 (m, 2 H), 3.36-3.26 (m, 3 H), 1.14 (d, J=6.8 Hz, 3 H). MS calculated for C₁₂H₁₄ClF₃N₂+H: 279, observed 279.

### Example 3.104

### Preparation of (S)-1-(4Chloro-3-trifluoromethyl-phenyl)-2-methyl-piperazine hydrochloride salt (Compound 104 HCl salt)

By the same general procedure as in Example 3.2, Compound 104 was obtained from the appropriate intermediates. ¹H NMR (400 MHz, CD₃OD) δ 7.50 (d, J=9.2 Hz, 1 H), 7.36 (d, J=2.8 Hz, 1 H), 7.25 (dd, J=8.8, 2.8 Hz, 1 H), 4.16-4.09 (m, 1 H), 3.52-3.41 (m, 3 H), 3.34-3.25 (m, 3 H), 1.13 (d, J=6.8 Hz, 3 H). MS calculated for C₁₂H₁₄ClF₃N₂+H: 279, observed 279.

### Example 3.105

### Preparation of (R)-2,4-Dimethyl-1-(3-trifluoromethyl-phenyl)-piperazine trifluoroacetic acid salt (Compound 105 TFA salt)

A solution of (S)-2-methyl-1-(3-trifluoromethyl-phenyl)-piperazine hydrochloride salt (130 mg, 1.1 mmol), paraformaldehyde (100 mg), acetic acid (100 μL, 1.7 mmol), and sodium cyanoborohydride (90 mg, 1.4 mmol) in 5 mL of MeOH was stirred at 65° C. for 8 hours. Afterwards, the crude mixture was concentrated to dryness, redissolved in 60 mL of CH₂Cl₂, and washed with 1 M K₂CO₃ (2×60 mL) and saturated aqueous NaCl (60 mL). The resulting organic solution was dried over MgSO₄, vacuum filtered, and concentrated to a brown oil. Purification by gradient reverse-phase HPLC (Phenomenex Luna 10μ C₁₈(2) column, acetonitrile-water with 0.1% TFA) afforded 70 mg (61%) of a clear oil as amixture of NMR conformers. ¹H NMR (400 MHz, CD₃OD) δ 7.57-7.44 (m, 2 H), 7.22-7.14 (m, 2 H), 4.46 (br s, 0.6 H), 3.71-3.55 (m, 2.7 H), 3.42-3.16 (m, 3.7 H), 2.98 (s, 3 H), 1.19 (d, J=6.8 Hz, 2 H), 0.97 (d, J=4.4 Hz, 1 H). MS calculated for C₁₃H₁₇F₃N₂+H: 259, observed: 259.

### Example 3.106

### Preparation of (S)-2,4-Dimethyl-1-(3-trifluoromethyl-phenyl)-piperazine trifluoroacetic acid salt (Compound 106 TFA salt)

By the same general procedure as in Example 3.105, Compound 106 was obtained from the appropriate intermediates. ¹H NMR (400 MHz, CD₃OD) δ 7.57-7.44 (m, 2 H), 7.22-7.14 (m, 2 H), 4.46 (br s, 0.6 H), 3.71-3.55 (m, 2.7 H), 3.42-3.16 (m, 3.7 H), 2.98 (s, 3 H), 1.19 (d, J=6.8 Hz, 2 H), 0.97 (d, J=4.4 Hz, 1 H). MS calculated for C₁₃H₁₇F₃N₂+H: 259, observed: 259.

### Example 3.107

### Preparation of (R)-2,4-Dimethyl-1-(4-trifluoromethyl-phenyl)-piperazine trifluoroacetic acid salt (Compound 107 TFA salt)

By the same general procedure as in Example 3.105, Compound 107 was obtained from the appropriate intermediates. ¹H NMR (400 MHz, CD₃OD) δ 7.65 (s, 0.4 H), 7.54 (d, J=8.4 Hz, 1.6 H), 7.35 (s, 0.4 H), 7.09 (d, J=8.4 Hz, 1:6 H), 4.52 (s, 1 H), 3.81 (d, J=14.0 Hz, 1 H), 3.65-3.56 (m, 2 H), 3.40-3.33 (m, 2 H), 3.19-3.15 (m, 1 H), 2.97 (s, 3 H), 1.22 (d, J=6.8 Hz, 2.4 H), 1.03 (s, 0.6 H). MS calculated for C₁₃H₁₇F₃N₂+H: 259, observed: 259.

### Example 3.108

### Preparation of (S)-2,4-Dimethyl-1-(4-trifluoromethyl-phenyl)-piperazine trifluoroacetic acid salt (Compound 108 TFA salt)

By the same general procedure as in Example 3.105, Compound 108 was obtained from the appropriate intermediates. ¹H NMR (400 MHz, CD₃OD) δ 7.65 (s, 0.4 H), 7.54 (d, J=8.4 Hz, 1.6 H), 7.35 (s, 0.4 H), 7.09 (d, J=8.4 Hz, 1.6 H), 4.52 (s, 1 H), 3.81 (d, J=14.0 Hz, 1 H), 3.65-3.56 (m, 2 H), 3.40-3.33 (m, 2 H), 3.19-3.15 (m, 1 H), 2.97 (s, 3 H), 1.22 (d, J=6.8 Hz, 2.4 H), 1.03 (s, 0.6 H). MS calculated for C₁₃H₁₇F₃N₂+H: 259, observed: 259.

### Example 3.109

### Preparation of (S)-1-(2-Fluoro-3-trifluoromethyl-phenyl)-2,4-dimethyl-piperazine trifluoroacetic acid salt (Compound 109 TFA salt)

By the same general procedure as in Example 3.105, Compound 109 was obtained from the appropriate intermediates. ¹H NMR (400 MHz, CD₃OD) δ 7.62 (t, J=6.7 Hz, 0.7 H), 7.56 (t, J=7.2 Hz, 0.7 H), 7.43-7.28 (m, 1.6 H), 4.09 (br s, 0.2 H), 3.63-3.41 (m, 3.3 H), 3.35-3.24 (m, 2.5 H), 3.02-2.89 (m, 4 H), 1.18 (d, J=6.8 Hz, 0.8 H), 0.98 (d, J=6.4 Hz, 2.2 H). MS calculated for C₁₃H₁₆F₄N₂+H: 277, observed: 277.

### Example 3.110

### Preparation of (S)-1-(4-Chloro-3-trifluoromethyl-phenyl)-2,4-dimethyl-piperazine trifluoroacetic acid salt (Compound 110 TFA salt)

By the same general procedure as in Example 3.105, Compound 110 was obtained from the appropriate intermediates. ¹H NMR (400 MHz, CD₃OD) δ 7.61-7.57 (m, 0.9 H), 7.46 (d, J=8.8 Hz, 0.7 H), 7.30 (appar d, 0.7 H), 7.19 (appar dd, J=8.4 Hz, 0.7 H), 4.45 (br s, 0.7 H), 3.77-3.56 (m, 3.3 H), 3.43-3.17 (m, 3 H), 2.98 (s, 3 H), 1.21 (d, J=6.8Hz, 1 H), 0.98 (d, J=5.2 Hz, 2 H). MS calculated for C₁₃H₁₆ClF₃N₂+H: 293, observed: 293.

### Example 3.111

### Preparation of (R)-1-(2-Chloro-5-trifluoromethyl-phenyl)-2,4-dimethyl-piperazine trifluoroacetic acid salt (Compound 111 TFA salt)

By the same general procedure as in Example 3.105, Compound 111 was obtained from the appropriate intermediates. ¹H NMR (400 MHz, CD₃OD) δ 7.79 (d, J=2.0 Hz, 0.1 H), 7.69 (d, J=8.4 Hz, 0.8 H), 7.64 (d, J=1.6 Hz, 0.9 H), 7.53 (dd, J=8.4, 1.6 Hz, 0.8 H), 7.46-7.41 (m, 0.2 H), 3.67-3.53 (m, 2.6 H), 3.49-3.48 (m, 0.3 H), 3.37-3.22 (m, 2.1 H), 3.13-2.91 (m, 5 H), 1.12 (d, J=6.8 Hz, 0.5 H), 0.95 (d, J=6.0 Hz, 2.5 H). MS calculated for C₁₃H₁₆ClF₃N₂+H: 293, observed: 293.

### Example 3.112

### Preparation of (S)-1-(2-Chloro-5-trifluoromethyl-phenyl)-2,4-dimethyl-piperazine trifluoroacetic acid salt (Compound 112 TFA salt)

By the same general procedure as in Example 3.105, Compound 112 was obtained from the appropriate intermediates. ¹H NMR (400 MHz, CD₃OD) δ 7.79 (d, J=2.0 Hz, 0.1 H), 7.69 (d, J=8.4 Hz, 0.8 H), 7.64 (d, J=1.6 Hz, 0.9 H), 7.53 (dd, J=8.4, 1.6 Hz, 0.8 H), 7.46-7.41 (m, 0.2 H), 3.67-3.53 (m, 2.6 H), 3.49-3.48 (m, 0.3 H), 3.37-3.22 (m, 2.1 H), 3.13-2.91 (m, 5 H), 1.12 (d, J=6.8 Hz, 0.5 H), 0.95 (d, J=6.0 Hz, 2.5 H). MS calculated for C₁₃H₁₆ClF₃N₂+H: 293, observed: 293.

### Example 3.113

### Preparation of (R)-1-(3,5-Bis-trifluoromethyl-phenyl)-2,4-dimethyl-piperazine trifluoroacetic acid salt (Compound 113 TFA salt)

By the same general procedure as in Example 3.105, Compound 113 was obtained from the appropriate intermediates. ¹H NMR (400 MHz, CD₃OD) δ 7.75 (br s, 0.5 H), 7.48 (s, 1.7 H), 7.37 (s, 0.8 H), 4.59 (s, 0.8 H), 3.86 (d, J=12.8 Hz, 0.9 H), 3.66-3.34 (m, 4.3 H), 3.25-3.16 (m, 1 H), 2.99 (s, 3 H), 1.24 (d, J=4.0 Hz, 2.6 H), 1.06 (s, 0.4 H). MS calculated for C₁₄H₁₆F₆N₂+H: 327, observed: 327.

### Example 3.114

### Preparation of (S)-1-(3,5-Bis-trifluoromethyl-phenyl)-2,4-dimethyl-piperazine trifluoroacetic acid salt (Compound 114 TFA salt)

By the same general procedure as in Example 3.105, Compound 114 was obtained from the appropriate intermediates. ¹H NMR (400 MHz, CD₃OD) δ 7.75 (br s, 0.5 H), 7.48 (s, 1.7 H), 7.37 (s, 0.8 H), 4.59 (s, 0.8 H), 3.86 (d, J=12.8 Hz, 0.9 H), 3.66-3.34 (m, 4.3 H), 3.25-3.16 (m, 1 H), 2.99 (s, 3 H), 1.24 (d, J=4.0 Hz, 2.6 H), 1.06 (s, 0.4 H). MS calculated for C₁₄H₁₆F₆N₂+H: 327, observed: 327.

### Example 3.115

### Preparation of (S)-1-(4-Fluoro-2-methyl-phenyl)-2,4-dimethyl-piperazine trifluoroacetic acid salt (Compound 115 TFA salt)

By the same general procedure as in Example 3.105, Compound 115 was obtained from the appropriate intermediates. ¹H NMR (400 MHz, DMSO-d6) δ 10.17 (s, 1 H), 7.21 (dd, J=8.8, 5.6 Hz, 1 H), 7.10 (dd, J=9.8, 3.0 Hz, 1 H), 7.02 (td, J=8.4, 3.1 Hz, 1 H), 3.51-3.42 (m, 2 H), 3.37-3.28 (m, 1 H), 3.22-3.12 (m, 1 H), 3.00-2.76 (m, 6 H), 2.28 (s, 3 H), 0.75 (d, J=6.0 Hz, 3 H). MS calculated for C₁₃H₁₉FN₂+H: 223, observed: 223.

### Example 3.116

### Preparation of (R)-1(2,3-Dichloro-phenyl)-2,4-dimethyl-piperazine trifluoroacetic acid salt (Compound 116 TFA salt)

By the same general procedure as in Example 3.105, Compound 116 was obtained from the appropriate intermediates. ¹H NMR (400 MHz, CD₃OD) δ 7.50-7.40 (m, 1 H), 7.36-7.28 (m, 2 H), 3.60-3.47 (m, 3 H), 3.40-3.14 (m, 2 H), 3.03-2.92 (m, 5 H), 1.10 (d, J=5.4 Hz, 0.5 H), 0.95 (d, J=6.0 Hz, 2.5 H). MS calculated for C₁₂H₁₆Cl₂N₂+H: 259, observed: 259.

### Example 3.117

### Preparation of (R)-1-(3,5-Dichloro-phenyl)-2,4-dimethyl-piperazine trifluoroacetic acid salt (Compound 117 TFA salt)

By the same general procedure as in Example 3.105, Compound 117 was obtained from the appropriate intermediates. ¹H NMR (400 MHz, CD₃OD) δ 7.18 (br s, 1 H), 6.95-6.90 (m, 2 H), 4.14 (s, 1 H), 3.67-3.52 (m, 4 H), 3.30-3.27 (m, 2 H), 2.96 (s, 3 H), 1.19 (s, 2.5 H), 1.03 (s, 0.5 H). MS calculated for C₁₂H₁₆Cl₂N₂+H: 259, observed: 259.

### Example 3.118

### Preparation of (R)-1-(3-Chloro-phenyl)-2,4-dimethyl-piperazine trifluoroacetic acid salt (Compound 118 TFA salt)

By the same general procedure as in Example 3.105, Compound 118 was obtained from the appropriate intermediates. ¹H NMR (400 MHz, CD₃OD) δ 7.35-7.15 (m, 2 H), 6.99 (s, 1 H), 6.90-6.75 (m, 1 H), 4.38 (s, 0.6 H), 3.65.3.48 (m, 2.4 H), 3.40-3.25 (m, 1 H), 3.27-3.09 (m, 3 H), 2.97 (s, 3 H), 1.16 (d, J=6.8 Hz, 2 H), 0.97 (br s, 1 H). MS calculated for C₁₂H₁₇ClN₂+H: 225, observed: 225.

### Example 3.119

### Preparation of (R)-1-(5-Chloro-2-fluoro-phenyl)-2,4-dimethyl-piperazine trifluoroacetic acid salt (Compound 119 TFA salt)

By the same general procedure as in Example 3.105, Compound 119 was obtained from the appropriate intermediates. ¹H NMR (400 MHz, CD₃OD) δ 7.35 (dd, J=6.8, 2.8 Hz, 0.8 H), 7.26 (ddd, J=8.8, 4.2, 2.6 Hz, 0.7 H), 7.18-7.05 (m, 1.5 H), 4.12-4.08 (m, 0.3 H), 3.60-3.46 (m, 3.4 H), 3.35-3.24 (m, 2.3 H), 2.97-2.91 (m, 4 H), 1.18 (d, J=7.2 Hz, 0.8 H), 0.98 (d, J=6.4 Hz, 2.2 H). MS calculated for C₁₂H₁₆ClFN₂+H: 243, observed: 243.

### Example 3.120

### Preparation of (S)-1-(2-Fluoro-phenyl)-2,4-dimethyl-piperazine trifluoroacetic acid salt (Compound 120 TFA salt)

By the same general procedure as in Example 3.105, Compound 120 was obtained from the appropriate intermediates. ¹H NMR (400 MHz, CD₃OD) δ 7.31-7.23 (m, 1.6 H), 7.19-7.07 (m, 2.4 H), 4.05 (s, 0.2 H), 3.56-3.45 (m, 3.4 H), 3.32-3.21 (m, 2.4 H), 2.97-2.89 (m, 4 H), 1.12 (d, J=6.8 Hz, 0.5 H), 0.95 (d, J=6.0 Hz, 2.5 H). MS calculated for C₁₂H₁₇FN₂+H: 209, observed: 209.

### Example 3.121

### Preparation of (S)-1-(2-Fluoro-4-trifluoromethyl-phenyl)-2,4-dimethyl-piperazine trifluoroacetic acid salt (Compound 121 TFA salt)

By the same general procedure as in Example 3.105, Compound 121 was obtained from the appropriate intermediates. ¹H NMR (400 MHz, DMSO-d6) δ 10.22 (s, 0.4 H), 9.90 (s, 0.6 H), 7.74-7.50 (m, 2.5 H), 7.26 (t, J=8.4 Hz, 0.5 H), 3.58-2.92 (m, 7 H), 2.51 (s, 3 H), 1.13 (d, J=6.8 Hz, 1.8 H), 0.92 (d, J=5.2 Hz, 1.2 H). MS calculated for C₁₃H₁₆F₄N₂+H: 277, observed: 277.

### Example 3.122

### Preparation of (S)-1-(2-Fluoro-5-trifluoromethyl-phenyl)-2,4-dimethyl-piperazine trifluoroacetic acid salt (Compound 122 TFA salt)

By the same general procedure as in Example 3.105, Compound 122 was obtained from the appropriate intermediates. ¹H NMR (400 MHz, DMSO-d6) δ 7.63-7.59 (m, 1.3 H), 7.40-7.29 (m, 1.7 H), 4.16 (s, 0.3 H), 3.70-3.48 (m, 3.3 H), 3.37-3.24 (m, 2.4 H), 2.98-2.91 (m, 4 H), 1.17 (d, J=7.2 Hz, 0.8 H), 0.97 (d, J=6.0 Hz, 2.2 H). MS calculated for C₁₃H₁₆F₄N₂+H: 277, observed: 277.

### Example 4

### Separation of Enantiomers for Selected Compounds of the Invention

The following Compounds were separated into their respective enantiomers using a Varian ProStar HPLC system with a 20 mm×250 mm Chiralcel OD chiral column, eluting with 0.2% diethylamine in various concentrations of isopropanol (IPA) in hexanes, see Table 5 below. In some cases, the separations were performed on the intermediate trifluoroacetamide protected amines.

TABLE 5

| | | Retention time | |
|---|---|---|---|
| | | for the free | |
| Compound | Enantiomer | amine (mins) | Conditions |
| Compound 15 | Enantiomer 1 | 21.1 | 3% IPA in hexane |
| | Enantiomer 2 | 25.3 | 9 mL/min |
| Compound 49 | Enantiomer 1 | 14.2 | 3% IPA in hexane |
| | Enantiomer 2 | 17.1 | 9 mL/min |

It is intended that each of the patents, applications, printed publications, and other published documents mentioned or referred to in this specification be herein incorporated by reference in their entirety.

Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

1. A compound of Formula (I): 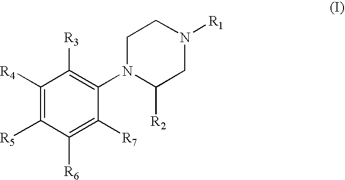
wherein:
R₁ is H or C₁₋₈ alkyl;
R₂ is C₂₋₄ alkenyl, C₁₋₄ alkyl or C₁₋₄ haloalkyl; and
R₃, R₄, R₅, R₆ and R₇ are each independently H, C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl, phenyl, and thiol; or
a pharmaceutically acceptable salt, hydrate and solvate thereof;
provided that the compound is not 1-(4-Chloro-phenyl)-2-methyl-piperazine; 1-(3,5-Difluoro-phenyl)-2-methyl-piperazine; 2-Methyl-1-(2-methylsulfanyl-phenyl)-piperazine; 4-Amino-3-fluoro-2-(2-methyl-piperazin-1-yl)-5-nitro-benzonitrile; 2-Methyl-1-phenyl-piperazine; 4-(2-Isopropyl-piperazin-1-yl)-2-trifluoromethyl-benzonitrile; 4-(2-Ethyl-piperazin-1-yl)-2-trifluoromethyl-benzonitrile; 4-(2-Methyl-piperazin-1-yl)-2-trifluoromethyl-benzonitrile; 1-(3-Chloro-phenyl)-2-methyl-piperazine; 2-Methyl-1-m-tolyl-piperazine; 4-(2-Methyl-piperazin-1-yl)-benzamide; 1-(2-Fluoro-phenyl)-2-methyl-piperazine; 4-(2-Methyl-piperazin-1-yl)-phenol; 1-(3-Methoxy-phenyl)-2-methyl-piperazine; 2-Methyl-1-(3-trifluoromethyl-phenyl)-piperazine; 1-(4-Methoxy-phenyl)-2-methyl-piperazine; 2-Methyl-1-p-tolyl-piperazine; 2,4-Dimethyl-1-phenyl-piperazine; 4-Chloro-5-(4-ethyl-2-methyl-piperazin-1-yl)-benzene-1,2-diamine; 4-Chloro-5-(4-ethyl-2-methyl-piperazin-1-yl)-2-nitro-phenylamine; 5-(4-Ethyl-2-methyl-piperazin-1-yl)-2-nitro-4-trifluoromethyl-phenylamine; and 5-(4-Ethyl-2-methyl-piperazin-1-yl)-4-methyl-2-nitro-phenylamine.

2. The compound according to claim 1 wherein R₁ is H.

3. The compound according to claim 1 wherein R₁ is C₁₋₈ alkyl.

4-8. (canceled)

9. The compound according to claim 1 wherein R₂ is C₂₋₄ alkenyl.

10. (canceled)

11. The compound according to claim 1 wherein R₂ is C₁₋₄ alkyl.

12. The compound according to claim 1 wherein R₂ is methyl.

13-16. (canceled)

17. The compound according to claim 1 wherein R₃, R₄, R₅, R₆ and R₇ are each independently selected from the group consisting of H, C₁₋₄ alkoxy, C₁₋₄ alkyl, cyano, C₁₋₄ haloalkoxy, C₁₋₄ haloalkyl and halogen.

18. (canceled)

19. The compound according to claim 17 wherein R₃, R₄, R₅, R₆ and R₇ are each independently selected from the group consisting of H, C₁₋₄ haloalkoxy, C₁₋₄ haloalkyl and halogen.

20. The compound according to claim 17 wherein R₃, R₄, R₅, R₆ and R₇ are each independently selected from the group consisting of H, CH₃, CH₂CH₃, CH(CH₃)₂, cyano, OCF₃, CF₃, F, Cl and Br.

21. The compound according to claim 17 wherein R₃, R₄, R₅, R₆ and R₇ are each independently selected from the group consisting of H, CF₃, F, Cl and Br.

22. The compound according to claim 1 wherein R₃ is H or F.

23. The compound according to claim 1 wherein R₄ is selected from the group consisting of H, cyano, F, Cl and Br.

24. The compound according to claim 1 wherein R₅ is selected from the group consisting of H, CH₃, CH(CH₃)₂, OCF₃, CF₃, F, Cl and Br.

25. The compound according to claim 1 wherein R₆ is selected from the group consisting of H, F, Cl and Br.

26. The compound according to claim 1 wherein R₇ is selected from the group consisting of H, CH₃, F, Cl and Br.

27. The compound of claim 1 selected from the group consisting of:
1-(2,3-Difluoro-phenyl)-2-ethyl-piperazine;
1-(3-Fluoro-phenyl)-2-ethyl-piperazine;
1-(4-Fluoro-phenyl)-2-ethyl-piperazine;
(R)-1-(3-Chloro-4-fluoro-phenyl)-2-methyl-piperazine;
(S)-1-(3-Chloro-4-fluoro-phenyl)-2-methyl-piperazine;
(R)-1-(3,4-Difluoro-phenyl)-2-methyl-piperazine;
(S)-1-(3,4-Difluoro-phenyl)-2-methyl-piperazine;
(R)-1-(3-Chloro-2-fluoro-phenyl)-2-methyl-piperazine;
(S)-1-(3-Chloro-2-fluoro-phenyl)-2-methyl-piperazine;
(R)-1-(4-Fluoro-phenyl)-2-methyl-piperazine;
(S)-1-(4-Fluoro-phenyl)-2-methyl-piperazine;
(R)-1-(3,4-Dichloro-phenyl)-2-methyl-piperazine;
(S)-1-(3,4-Dichloro-phenyl)-2-methyl-piperazine;
(R)-1-(3-Chloro-4-methyl-phenyl)-2-methyl-piperazine;
(S)-1-(3-Chloro-4-methyl-phenyl)-2-methyl-piperazine;
(R)-1-(3,4-Difluoro-phenyl)-2-methyl-piperazine;
(S)-1-(3,4-Difluoro-phenyl)-2-methyl-piperazine;
(R)-1-(3,5-Dichloro-phenyl)-2-methyl-piperazine;
(S)-1-(3,5-Dichloro-phenyl)-2-methyl-piperazine;
(R)-1-(2,5-Di fluoro-phenyl)-2-methyl-piperazine;
(S)-1-(2,5-Difluoro-phenyl)-2-methyl-piperazine;
(R)-1-(4-Chloro-3-fluoro-phenyl)-2-methyl-piperazine;
(S)-1-(4-Chloro-3-fluoro-phenyl)-2-methyl-piperazine;
(R)-1-(3-Chloro-2-methyl-phenyl)-2-methyl-piperazine;
(S)-1-(3-Chloro-2-methyl-phenyl)-2-methyl-piperazine;
(R)-1-(5-Chloro-2-fluoro-phenyl)-2-methyl-piperazine;
(S)-1-(5-Chloro-2-fluoro-phenyl)-2-methyl-piperazine;
(R)-1-(5-Chloro-2-methyl-phenyl)-2-methyl-piperazine;
(S)-1-(5-Chloro-2-methyl-phenyl)-2-methyl-piperazine;
1-(3-Chloro-4-fluoro-phenyl)-2-ethyl-piperazine;
1-(3-Chloro-phenyl)-2-ethyl-piperazine;
1-(4-Chloro-phenyl)-2-ethyl-piperazine;
1-(3,4-Difluoro-phenyl)-2-ethyl-piperazine and
(R)-1-(5-Chloro-2-fluoro-phenyl)-2-ethyl-piperazine;
or a pharmaceutically acceptable salt, hydrate and solvate thereof.

28. The compound of claim 1 selected from the group consisting of:
(R)-1-(2-Fluoro-5-trifluoromethyl-phenyl)-2-methyl-piperazine;
(S)-1-(2-Fluoro-5-trifluoromethyl-phenyl)-2-methyl-piperazine;
(R)-1-(4-Chloro-2-fluoro-phenyl)-2-methyl-piperazine;
(S)-1-(4-Chloro-2-fluoro-phenyl)-2-methyl-piperazine;
(R)-1-(3-Chloro-5-fluoro-phenyl)-2-methyl-piperazine;
(S)-1-(3-Chloro-5-fluoro-phenyl)-2-methyl-piperazine;
(R)-1-(3-Fluoro-phenyl)-2-methyl-piperazine;
(S)-1-(3-Fluoro-phenyl)-2-methyl-piperazine;
(R)-1-(2-Fluoro-4-trifluoromethyl-phenyl)-2-methyl-piperazine;
(S)-1-(2-Fluoro-4-trifluoromethyl-phenyl)-2-methyl-piperazine;
(R)-1-(2-Chloro-3-fluoro-phenyl)-2-methyl-piperazine;
(S)-1-(2-Chloro-3-fluoro-phenyl)-2-methyl-piperazine;
(R)-1-(2-Fluoro-5-methyl-phenyl)-2-methyl-piperazine;
(S)-1-(2-Fluoro-5-methyl-phenyl)-2-methyl-piperazine;
(R)-1-(4-Fluoro-biphenyl-3-yl)-2-methyl-piperazine;
(S)-1-(4-Fluoro-biphenyl-3-yl)-2-methyl-piperazine;
(R)-1-(2,5-Difluoro-4-methoxy-phenyl)-2-methyl-piperazine;
(S)-1-(2,5-Difluoro-4-methoxy-phenyl)-2-methyl-piperazine;
(R)-1-(2-Fluoro-4-methyl-phenyl)-2-methyl-piperazine;
(S)-1-(2-Fluoro-4-methyl-phenyl)-2-methyl-piperazine;
(R)-1-(2-Chloro-5-fluoro-phenyl)-2-methyl-piperazine;
(S)-1-(2-Chloro-5-fluoro-phenyl)-2-methyl-piperazine;
(R)-1-(2-Chloro-4-fluoro-phenyl)-2-methyl-piperazine;
(S)-1-(2-Chloro-4-fluoro-phenyl)-2-methyl-piperazine;
(R)-1-(2,4-Dichloro-phenyl)-2-methyl-piperazine;
(S)-1-(2,4-Dichloro-phenyl)-2-methyl-piperazine;
(R)-1-(2,5-Dichloro-phenyl)-2-methyl-piperazine;
(S)-1-(2,5-Dichloro-phenyl)-2-methyl-piperazine;
(R)-1-(3,5-Bis-trifluoromethyl-phenyl)-2-methyl-piperazine;
(S)-1-(3,5-Bis-trifluoromethyl-phenyl)-2-methyl-piperazine;
(R)-1-(4-Fluoro-2-methyl-phenyl)-2-methyl-piperazine;
(S)-1-(4-Fluoro-2-methyl-phenyl)-2-methyl-piperazine;
(R)-1-(2-Chloro-phenyl)-2-methyl-piperazine;
(S)-1-(2-Chloro-phenyl)-2-methyl-piperazine;
(R)-1-(2,3-Dichloro-phenyl)-2-methyl-piperazine;
(R)-1-(2,3-Dichloro-phenyl)-2-methyl-piperazine;
(R)-1-(2,6-Dichloro-phenyl)-2-methyl-piperazine;
(R)-1-(2,6-Dichloro-phenyl)-2-methyl-piperazine;
(R)-1-(2-Chloro-5-trifluoromethyl-phenyl)-2-methyl-piperazine;
(R)-1-(2-Chloro-5-trifluoromethyl-phenyl)-2-methyl-piperazine;
(R)-2-Methyl-1-(4-trifluoromethyl-phenyl)-piperazine;
(S)-2-Methyl-1-(4-trifluoromethyl-phenyl)-piperazine;
(R)-1-(2-Fluoro-3-trifluoromethyl-phenyl)-2-methyl-piperazine;
(S)-1-(2-Fluoro-3-trifluoromethyl-phenyl)-2-methyl-piperazine;
(R)-1-(3-Fluoro-5-trifluoromethyl-phenyl)-2-methyl-piperazine;
(R)-1-(3-Fluoro-5-trifluoromethyl-phenyl)-2-methyl-piperazine;
(R)-1-(4-Chloro-3-trifluoromethyl-phenyl)-2-methyl-piperazine;
(S)-1-(4-Chloro-3-trifluoromethyl-phenyl)-2-methyl-piperazine; and
(R)-2,4-Dimethyl-1-(3-trifluoromethyl-phenyl)-piperazine;
or a pharmaceutically acceptable salt, hydrate and solvate thereof.

29. The compound of claim 1 selected from the group consisting of:
1-(2-Bromo-phenyl)-2-vinyl-piperazine;
1-(4-Chloro-phenyl)-2-vinyl-piperazine;
1-(3-Fluoro-phenyl)-2-vinyl-piperazine;
1-(3-Chloro-4-fluoro-phenyl)-2-vinyl-piperazine;
1-(3-Chloro-phenyl)-2-vinyl-piperazine;
1-(3-Bromo-phenyl)-2-vinyl-piperazine;
1-(3,5-Dichloro-phenyl)-2-vinyl-piperazine;
1-(2-Bromo-4-isopropyl-phenyl)-2-vinyl-piperazine;
1-(2-Bromo-4-trifluoromethoxy-phenyl)-2-vinyl-piperazine;
1-(2-Bromo-4-trifluoromethyl-phenyl)-2-vinyl-piperazine;
3-(2-Vinyl-piperazin-1-yl)-benzonitrile;
1-(3,5-Difluoro-phenyl)-2-vinyl-piperazine;
1-o-Tolyl-2-vinyl-piperazine and
1-(2,3-Difluoro-phenyl)-2-vinyl-piperazine;
or a pharmaceutically acceptable salt, hydrate and solvate thereof.

30. The compound according to claim 1 wherein said compound is an R enantiomer.

31. The compound according to claim 1 wherein said compound is an S enantiomer.

32. A pharmaceutical composition comprising a pharmaceutical acceptable carrier in combination with at least one compound according to Formula (I): 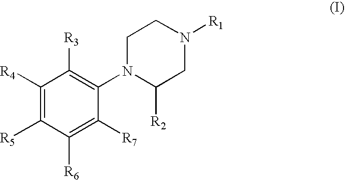
wherein:
R₁ is H or C₁₋₈ alkyl;
R₂ is C₂₋₄ alkenyl, C₁₋₄ alkyl or C₁₋₄ haloalkyl; and
R₃, R₄, R₅, R₆ and R₇ are each independently H, C₁₋₄ acyl, C₁₋₄ acyloxy, C₁₋₄ acylthioxy, C₂₋₄ alkenyl, C₁₋₄ alkoxy, C₁₋₄ alkyl, C₁₋₄ alkylcarboxamido, C₁₋₄ alkylsulfinyl, C₁₋₄ alkylsulfonamide, C₁₋₄ alkylsulfonyl, C₁₋₄ alkylthio, amino, C₁₋₄ alkylamino, carbo-C₁₋₄-alkoxy, carboxamide, cyano, C₂₋₆ dialkylamino, C₁₋₄ haloalkoxy, C₁₋₄ haloalkyl, C₁₋₄ haloalkylsulfinyl, C₁₋₄ haloalkylsulfonyl, C₁₋₄ haloalkylthio, halogen, hydroxyl, phenyl, and thiol; or
a pharmaceutically acceptable salt, hydrate and solvate thereof.

33. A method of modulating a 5HT_{2C} receptor comprising contacting said receptor with a therapeutically effective amount of a compound as in claim 1.

34. The method according to claim 33 wherein said compound is an agonist of said receptor.

35. A method of prophylaxis or treatment of disorders of the central nervous system; damage to the central nervous system; cardiovascular disorders; gastrointestinal disorders; diabetes insipidus or sleep apnea comprising administering to an individual in need of such prophylaxis or treatment a therapeutically effective amount of a compound according to claim 1 or a pharmaceutical composition according to claim 32.

36. The method according to claim 35 wherein the disorders of the central nervous system are selected the group consisting of depression, atypical depression, bipolar disorders, anxiety disorders, obsessive-compulsive disorders, social phobias or panic states, sleep disorders, sexual dysfunction, psychoses, schizophrenia, migraine and other conditions associated with cephalic pain or other pain, raised intracranial pressure, epilepsy, personality disorders, Alzheimer disease, age-related behavioral disorders, behavioral disorders associated with dementia, organic mental disorders, mental disorders in childhood, aggressivity, age-related memory disorders, chronic fatigue syndrome, drug and alcohol addiction, obesity, bulimia, anorexia nervosa and premenstrual tension.

37. The method according to claim 36 wherein the disorder of the central nervous system is obesity.

38. (canceled)

39. The method according to claim 36 wherein the sexual dysfunction is Male erectile dysfunction.

40-44. (canceled)

45. The method according to claim 37 or 39 wherein said individual is a human.

46. A method of decreasing food intake of an individual comprising administering to said individual a therapeutically effective amount of a compound according to claim 1 or a pharmaceutical composition according to claim 32.

47. (canceled)

48. The method according to claim 46 wherein said individual is a human.

49. A method of inducing satiety in an individual comprising administering to said individual a therapeutically effective amount of a compound according to claim 1 or a pharmaceutical composition according to claim 32.

50. (canceled)

51. The method according to claim 49 wherein said individual is a human.

52. A method of controlling weight gain of an individual comprising administering to said individual suffering from weight control a therapeutically effective amount of a compound according to claim 1 or a pharmaceutical composition according to claim 32.

53. (canceled)

54. The method according to claim 52 wherein said individual is a human.

55-58. (canceled)

59. A method of producing a pharmaceutical composition comprising admixing at least one compound according to claim 1 and a pharmaceutically acceptable carrier.

60-78. (canceled)

79. The compound according to claim 1 wherein:
R₁ is H, methyl, ethyl, n-propyl, iso-propyl or n-butyl;
R₂ is a vinyl, methyl, ethyl, n-propyl, C₁₋₄ haloalkyl or —CF₃;
R₃ is H or F;
R₄ is selected from the group consisting of H, cyano, F, Cl and Br;
R₅ is selected from the group consisting of H, CH₃, CH(CH₃)₂, OCF₃, CF₃, F, Cl and Br;
R₆ is selected from the group consisting of H, F, Cl and Br; and
R₇ is selected from the group consisting of H, CH₃, F, Cl and Br.

80. A method of treating a 5HT_{2C} receptor associated disorder comprising administering to an individual in need of such treatment an effective amount of a compound according to claim 1, or a pharmaceutical composition according to claim 32.
